(19) United States
(12) Patent Application Publication
(10) Pub. No.: US 2025/0354180 A1
(43) Pub. Date: Nov. 20, 2025

(54) ENZYMES, CELLS, AND METHODS FOR PRODUCING CIS-3 HEXENOL

(71) Applicant: MANUS BIO INC., Waltham, MA (US)

(72) Inventors: Arthur J. SHAW, Waltham, MA (US); John Carl SCHULTZ, Waltham, MA (US); Yannick BRUNET, Waltham, MA (US); Ryan A. PHILIPPE, Waltham, MA (US); Ajikumar PARAYIL KUMARAN, Waltham, MA (US); Christine Nicole S. SANTOS, Waltham, MA (US); Thomas E. WOOD, Waltham, MA (US); Chin Giaw LIM, Waltham, MA (US)

(21) Appl. No.: 18/864,750

(22) PCT Filed: May 12, 2023
(86) PCT No.: PCT/US2023/066949

§ 371 Date: Nov. 11, 2024

Related U.S. Application Data
(60) Provisional application No. 63/341,148, filed on May 12, 2022.

(51) Int. Cl. C12P 7/04; C12N 9/00; C12N 9/02; C12N 9/04; C12N 9/16; C12N 9/90; C12N 15/52

(52) U.S. Cl. CPC C12P 7/04; C12N 9/0006; C12N 9/0008; C12N 9/001; C12N 9/16; C12N 9/90; C12N 9/93; C12N 15/52; C12Y 101/01001; C12Y 102/01084; C12Y 103/08001; C12Y 301/02014; C12Y 503/03008; C12Y 602/01001

(57) ABSTRACT

The present disclosure relates, in part, to microbial hosts capable of synthesizing cis-3-hexenol, cis-3-hexenal, trans-3-hexenol, trans-3-hexenal, trans-2-hexenal, cis-2-hexenal and related compounds from hexanoic acid and methods for the preparation of cis-3-hexenol, cis-3-hexenal, trans-3-hexenol, trans-3-hexenal, trans-2-hexenal, cis-2-hexenal and related compounds.

### PRIORITY

This application claims the benefit of, and priority to, U.S. Provisional Application No. 63/341,148, filed May 12, 2022, which is hereby incorporated by reference in its entirety.

### SEQUENCE LISTING

The instant application contains a sequence listing, which has been submitted in XML format via EFS-Web. The contents of the XML copy named “MAN-035PC_107590-5034_Sequence_Listing,” which was created on May 11, 2023, and is 116,582 bytes in size, and the contents of which are incorporated herein by reference in their entirety.

### BACKGROUND

Cis-3-hexenol, also known as (Z)-hex-3-en-1-ol or leaf alcohol is a colorless oily liquid, which is emitted by green plants upon mechanical damage or general stress. It has an odor of freshly cut green grass and leaves. Cis-3-hexenol is a valuable compound that is used as a fragrance or flavorant. For example, cis-3-hexenol is used in tea, fruit flavors (apple and other fruit flavours) and vegetable flavors, as well as in perfumery.

Cis-3-hexenol can be isolated from plant sources or synthesized from unsaturated fatty acids. However, more sustainable, scalable, and/or cost-effective methods for preparing cis-3-hexenol are desired.

### BRIEF DESCRIPTION OF THE DRAWINGS

FIG. 1 shows a novel biosynthetic scheme for cis-3-hexenol production. Hexanoic acid, which is produced from acetyl-CoA via successive action of acetyl-CoA carboxylase and type I/type II fatty acid synthase complex, is converted by acyl-CoA synthetase (ACS) to hexanoyl-CoA, which is converted by a short chain acyl-CoA oxidase to trans-2-hexenoyl-CoA, which is converted by a enoyl-CoA isomerase (ECI) to cis-3-hexenoyl-CoA, which is converted by a fatty acyl-CoA reductase (FAR) to a cis-3-hexenal, which is in turn converted by an alcohol dehydrogenase (ADH) to cis-3-hexenol.

FIG. 2A and FIG. 2B show a screen of acyl-CoA synthetase (ACS) enzymes for conversion of hexanoic acid to hexanoyl-CoA. FIG. 2A shows the activities of various ACS enzymes in Yarrowia lipolytica extracts. FIG. 2B shows a representative liquid chromatography-mass spectrometry (LC-MS)-based detection of hexanoyl-CoA produced by an extract of Yarrowia lipolytica expressing an ACS enzyme. A hexanoyl-CoA standard is also shown.

FIG. 3A and FIG. 3B show a screen of acyl-CoA oxidase (AOX) enzymes for conversion of hexanoyl-CoA to trans-2-hexenoyl-CoA. FIG. 3A shows the activities of various AOX enzymes in Yarrowia lipolytica extracts. FIG. 3B shows a representative liquid chromatography-mass spectrometry (LC-MS)-based detection of hexanoyl-CoA and trans-2 hexanoyl-CoA produced by an extract of Yarrowia lipolytica expressing an AOX enzyme.

FIG. 4 shows the screen of alcohol dehydrogenase (ADH) enzymes in Yarrowia phangngensis (Yph) for conversion of cis-3-hexenal to cis-3-hexenol. A media-only control is shown on the left hand side.

FIG. 5A and FIG. 5B show the evaluation of the degree of selectivity for cis-3 vs trans-2-hexenal for alcohol dehydrogenase enzymes. FIG. 5A shows the biochemical reactions producing cis-3-hexenol and trans-2-hexenol. FIG. 5B shows the activities of Yarrowia lipolytica strains expressing recombinant ScADH or AtCHR genes and supplemented with cis-3-hexenal, trans-2-hexenal, or both cis-3-hexenal and trans-2-hexenal.

FIG. 6A and FIG. 6B show the evaluation of enoyl-CoA isomerase (ECI) activity in vitro. FIG. 6A shows the biochemical step for producing cis-3-hexenoyl-CoA from trans-2-hexenoyl-CoA in the synthetic pathway for producing cis-3-hexenol. FIG. 6B shows the conversion of trans-2-hexenoyl-CoA to cis-3-hexenoyl-CoA, as measured by LC-MS. ECI1 to ECI11, ECI14, and ECI15 are cell free extracts of strains expressing thirteen different enoyl-CoA isomerases (ECI).

FIG. 7A to FIG. 7C show the evaluation of the fatty acyl-CoA reductase (FAR) activity in vitro. FIG. 7A shows the biochemical step for producing cis-3-hexenal from cis-3-hexenoyl-CoA in the synthetic pathway for producing cis-3-hexenol. FIG. 7B shows the results of a screen of cell extracts expressing a panel of fatty acyl-CoA reductases (FAR) genes that were able to reduce NAD+, as measured by absorbance at 340 nm, upon the addition of cis-3-hexenal and CoA. FIG. 7C shows the results of a screen of cell extracts expressing a panel of fatty acyl-CoA reductases (FAR) genes that were able to reduce NADP+, as measured by absorbance at 340 nm, upon the addition of cis-3-hexenal and CoA.

FIG. 8A and FIG. 8B show the production of cis-3-hexenol from hexanoic acid by Yarrowia lipolytica strain having deletions of aldehyde dehydrogenase genes overexpressed in the strains that carry the recombinant cis-3-hexenol pathway. FIG. 8A shows the titers of various alcohols in strains having the deletion of YALI0C03025g or YALI0F04444g. FIG. 8B shows the GC-FID and GC-MS (inset) profiles demonstrating the production of cis-3-hexenol.

FIG. 9 shows the production of cis-3-hexenol from hexanoic acid by Escherichia coli in vivo, and improvement of titers through evaluation of FAR homologs.

FIG. 10A and FIG. 10B show the production of cis-3-hexenol from hexanoic acid by Escherichia coli strains expressing an expanded panel of FAR enzymes.

FIG. 11 shows a screen to evaluate ECI enzyme selectivity in the hexanoic acid to cis-3-hexenol bioconversion pathway in Escherichia coli.

FIG. 12 demonstrates that increasing ACS-AOX expression increased unsaturated six-carbon carboxylic acid production, while high ECI15 expression increased the proportion of off-pathway trans-3-hexenoic acid.

FIG. 13 shows a screen of ECI16-ECI20 for isomerization activity in the cis-3-hexenol bioconversion pathway in Escherichia coli.

FIG. 14 demonstrates that high aeration increases cis-3-hexenol and cis-3-hexenoic acid titers in the hexanoic bioconversion pathway in Escherichia coli.

FIG. 15 demonstrates that cultivation of Escherichia coli expressing the cis-3-hexenol bioconversion metabolic pathway at 37° C. modulates production of cis-3-hexenol in comparison to growth at 30° C.

FIG. 16 demonstrates the in vivo production of hexanoic acid in E. coli.

FIG. 17 shows the GC-MS chromatogram of culture extracts of Escherichia coli expressing the cis-3-hexenol production pathway. Corresponding ion spectra for labeled peaks are shown.

FIG. 18 demonstrates that overexpression of native ADH genes impacts unsaturated six-carbon alcohol production.

FIG. 19 demonstrates that ENR1 increases cis-3-hexenoic acid production over the native ENR FabI.

### DETAILED DESCRIPTION

The present disclosure is based, in part, on development of a fully synthetic biosynthetic pathway for the biosynthesis of cis-3-hexenol and related compounds. Accordingly, in various aspects, the present disclosure provides methods for making cis-3-hexenol and related compounds, and enzymes and host cells for use in these methods. The present disclosure provides engineered host cells for producing cis-3-hexenol and related products by microbial fermentation or bioconversion. The disclosure further provides methods of making products containing cis-3-hexenol and related compounds, including fragrances, cosmetics, food products, beverages, flavors, food additives, among others. Such products can be made at reduced cost and more sustainable fashion by virtue of this disclosure.

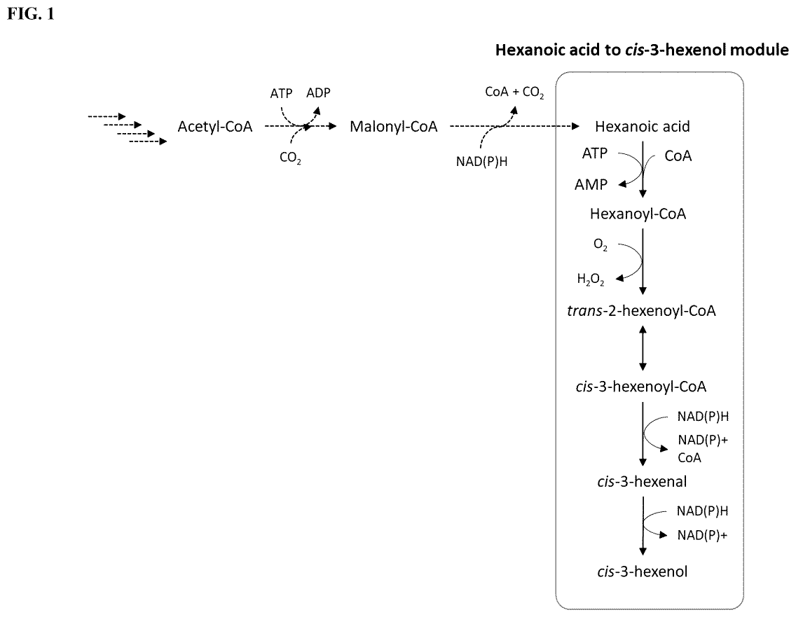

Disclosed herein is a novel synthetic heterologous biosynthetic pathway designed to biosynthesize cis-3-hexenol from hexanoic acid. In some embodiments, hexanoic acid is provided externally to the culture. In some embodiments, hexanoic acid is generated from the intermediates of glycolysis and/or the pentose phosphate pathway. The biosynthetic pathway for cis-3-hexenol is illustrated in FIG. 1.

Cis-3-hexenol, which is also known as leaf alcohol, is ubiquitous in the leaves of most plants. It is biosynthesized in response to wounds caused by pest infestation and grazing by animals. Cis-3-hexenol, being volatile, easily becomes airborne and serves as a signaling molecule that triggers defense responses against the insults that produced it. Farag et al., (Z)-3-Hexenol induces defense genes and downstream metabolites in maize, Planta 2005; 220(6):900-9; Sugimoto et al., Intake and transformation to a glycoside of (Z)-3-hexenol from infested neighbors reveals a mode of plant odor reception and defense, Proc Natl Acad Sci USA 2014; 111(19):7144-9; Liao et al., Herbivore-Induced (Z)-3-Hexen-1-ol is an Airborne Signal That Promotes Direct and Indirect Defenses in Tea (Camellia sinensis) under Light, J Agric Food Chem. 2021; 69(43):12608-12620. Natural biosynthesis of cis-3-hexenol starts from linolenic acid. Lipoxygenase oxidizes position 13 of linolenic acid using dioxygen as a substrate to produce linolenic acid 13-hydroperoxide. Linolenic acid 13-hydroperoxide is cleaved by hydroperoxide lyase (HPL) at the C12-C13 bond to produce two carbonyl compounds: cis-3-hexenal and a C₁₂-oxo-acid. Cis-3-hexenal is reduced to form cis-3-hexenol. Mwenda and Matsui, The importance of lipoxygenase control in the production of green leaf volatiles by lipase-dependent and independent pathways, Plant Biotechnology 2014; 31 (5): 445-452; Hatanaka, The biogeneration of green odor by green leaves, 1993; Phytochemistry, 34: 1201-1218; Hatanaka et al., The biogeneration of green odour by green leaves and it's physiological functions—past, present and future, Z. Naturforsch. C-A J. Biosci., 1995; 50: 467-472; Matsui, Green leaf volatiles: hydroperoxide lyase pathway of oxylipin metabolism, Curr Opin Plant Biol 2006; 9: 274-280; Matsui et al., Differential metabolisms of green leaf volatiles in injured and intact parts of a wounded leaf meet distinct ecophysiological requirements, PLOS One 2012; 7(4):e36433. While this pathway is suitable for plants to rapidly produce cis-3-hexenol in response to a wound caused by infestation or grazing, this biosynthetic pathway is not suitable for industrial production from cis-3-hexenol at least because it starts from a complex fatty acid-linolenic acid-compared to the simpler desired product and because it produces an oxo-acid byproduct. Therefore, a novel, fully synthetic, non-naturally existing biosynthetic pathway was developed for biosynthesis of cis-3-hexenol, and related compounds, from hexanoic acid.

Accordingly, in one aspect, the present disclosure relates to a microbial host cell producing cis-3-hexenol from hexanoic acid, the microbial cell expressing a recombinant biosynthetic pathway comprising: (a) an acyl-CoA synthetase (ACS) converting hexanoic acid to hexanoyl-CoA; (b) a short chain acyl-CoA oxidase (AOX) converting the hexanoyl-CoA to trans-2-hexenoyl-CoA; (c) optionally, an enoyl-CoA isomerase (ECI) converting the trans-2-hexenoyl-CoA to cis-3-hexenoyl-CoA; (d) a fatty acyl-CoA reductase (FAR) converting the cis-3-hexenoyl-CoA to cis-3-hexenal; and (e) optionally, an alcohol dehydrogenase (ADH) converting the cis-3-hexenal to cis-3-hexenol. In some embodiments, the enzymatic conversions of (c) and/or (d) are conducted wholly or partly by native enzymes of the host cell.

In one aspect, the present disclosure relates to a microbial host cell expressing a recombinant biosynthetic pathway comprising an acyl-CoA synthetase (ACS), wherein the microbial cell and/or the ACS is capable of converting hexanoic acid to hexanoyl-CoA. Any ACS that is capable of converting hexanoic acid to hexanoyl-CoA can be employed in various embodiments. In some embodiments, the ACS uses ATP as a source of energy.

In some embodiments, the ACS comprises an amino acid sequence that is at least 70% identical to an amino acid sequence selected from SEQ ID NOs: 1-7. In some embodiments, the ACS comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 1-7. In some embodiments, the ACS comprises an amino acid sequence having from 1 to 20, from 1 to 10, or from 1 to 5 amino acid modifications independently selected from substitutions, insertions, and deletions with respect to an amino acid sequence selected from SEQ ID NOs: 1-7.

In some embodiments, the ACS comprises an amino acid sequence that is at least 70% identical to an amino acid sequence selected from SEQ ID NO: 2, SEQ ID NO: 3 and SEQ ID NO: 6. In some embodiments, the ACS comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NO: 2, SEQ ID NO: 3 and SEQ ID NO: 6. In some embodiments, the ACS comprises an amino acid sequence having from 1 to 20, from 1 to 10, or from 1 to 5 amino acid modifications independently selected from substitutions, insertions, and deletions with respect to an amino acid sequence selected from SEQ ID NO: 2, SEQ ID NO: 3 and SEQ ID NO: 6.

In some embodiments, the ACS comprises an amino acid sequence that is at least 70% identical to the amino acid sequence of SEQ ID NO: 2. In some embodiments, the ACS comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 2. In some embodiments, the ACS comprises an amino acid sequence having from 1 to 20, from 1 to 10, or from 1 to 5 amino acid modifications independently selected from substitutions, insertions, and deletions with respect to the amino acid sequence of SEQ ID NO: 2.

In some embodiments, the ACS comprises an amino acid sequence that is at least 70% identical to the amino acid sequence of SEQ ID NO: 3. In some embodiments, the ACS comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 3. In some embodiments, the ACS comprises an amino acid sequence having from 1 to 20, from 1 to 10, or from 1 to 5 amino acid modifications independently selected from substitutions, insertions, and deletions with respect to the amino acid sequence of SEQ ID NO: 3.

In some embodiments, the ACS comprises an amino acid sequence that is at least 70% identical to the amino acid sequence of SEQ ID NO: 6. In some embodiments, the ACS comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 6. In some embodiments, the ACS comprises an amino acid sequence having from 1 to 20, from 1 to 10, or from 1 to 5 amino acid modifications independently selected from substitutions, insertions, and deletions with respect to the amino acid sequence of SEQ ID NO: 6.

In one aspect, the present disclosure relates to a microbial host cell expressing a recombinant biosynthetic pathway comprising a short chain acyl-CoA oxidase (AOX), wherein the microbial cell and/or the AOX is capable of converting the hexanoyl-CoA to trans-2-hexenoyl-CoA. Any AOX that is capable of converting the hexanoyl-CoA to trans-2-hexenoyl-CoA may be employed in various embodiments. In some embodiments, the AOX uses molecular oxygen (O₂) as a source of electrons. In some embodiments, the AOX produces hydrogen peroxide as a byproduct. In some embodiments, the microbial host cell further expresses or overexpresses a catalase.

In some embodiments, the AOX comprises an amino acid sequence that is at least 70% identical to an amino acid sequence selected from SEQ ID NOs: 8-20. In some embodiments, the AOX comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 8-20. In some embodiments, the AOX comprises an amino acid sequence having from 1 to 20, from 1 to 10, or from 1 to 5 amino acid modifications independently selected from substitutions, insertions, and deletions with respect to an amino acid sequence selected from SEQ ID NOs: 8-20.

In some embodiments, the AOX comprises an amino acid sequence that is at least 70% identical to the amino acid sequence of SEQ ID NO: 14. In some embodiments, the AOX comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 14. In some embodiments, the AOX comprises an amino acid sequence having from 1 to 20, from 1 to 10, or from 1 to 5 amino acid modifications independently selected from substitutions, insertions, and deletions with respect to the amino acid sequence of SEQ ID NO: 14.

In some embodiments, the AOX comprises an amino acid sequence that is at least 70% identical to the amino acid sequence of SEQ ID NO: 11. In some embodiments, the AOX comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 11. In some embodiments, the AOX comprises an amino acid sequence having from 1 to 20, from 1 to 10, or from 1 to 5 amino acid modifications independently selected from substitutions, insertions, and deletions with respect to the amino acid sequence of SEQ ID NO: 11.

In some embodiments, the AOX comprises an amino acid sequence that is at least 70% identical to the amino acid sequence of SEQ ID NO: 12. In some embodiments, the AOX comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 12. In some embodiments, the AOX comprises an amino acid sequence having from 1 to 20, from 1 to 10, or from 1 to 5 amino acid modifications independently selected from substitutions, insertions, and deletions with respect to the amino acid sequence of SEQ ID NO: 12.

In one aspect, the present disclosure relates to a microbial host cell expressing a recombinant biosynthetic pathway comprising an enoyl-CoA isomerase (ECI), wherein the microbial cell and/or the ECI is capable of converting trans-2-hexenoyl-CoA to cis-3-hexenoyl-CoA. In some embodiments, the ECI is capable of interconverting trans-2-hexenoyl-CoA and cis-3-hexenoyl-CoA. Any ECI that is capable of converting trans-2-hexenoyl-CoA to cis-3-hexenoyl-CoA may be employed in various embodiments.

In some embodiments, the ECI comprises an amino acid sequence that is at least 70% identical to an amino acid sequence selected from SEQ ID NOs: 21-35 and 73-77. In some embodiments, the ECI comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 21-35 and 73-77. In some embodiments, the ECI comprises an amino acid sequence having from 1 to 20, from 1 to 10, or from 1 to 5 amino acid modifications independently selected from substitutions, insertions, and deletions with respect to an amino acid sequence selected from SEQ ID NOs: 21-35 and 73-77.

In some embodiments, the microbial host cell is a yeast (without limitation, e.g., Yarrowia lipolytica), and the ECI comprises an amino acid sequence that is at least 70% identical to an amino acid sequence selected from SEQ ID NOs: 21-35. In some embodiments, the microbial host cell is a yeast (without limitation, e.g., Yarrowia lipolytica), and the ECI comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 21-35. In some embodiments, the microbial host cell is a yeast (without limitation, e.g., Yarrowia lipolytica), and the ECI comprises an amino acid sequence having from 1 to 20, from 1 to 10, or from 1 to 5 amino acid modifications independently selected from substitutions, insertions, and deletions with respect to an amino acid sequence selected from SEQ ID NOs: 21-35.

In some embodiments, the microbial host cell is a bacterium (without limitation, e.g., Escherichia coli), and the ECI comprises an amino acid sequence that is at least 70% identical to an amino acid sequence selected from SEQ ID NOs: 73-77. In some embodiments, the microbial host cell is a bacterium (without limitation, e.g., Escherichia coli), and the ECI comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 73-77. In some embodiments, the microbial host cell is a bacterium (without limitation, e.g., Escherichia coli), and the ECI comprises an amino acid sequence having from 1 to 20, from 1 to 10, or from 1 to 5 amino acid modifications independently selected from substitutions, insertions, and deletions with respect to an amino acid sequence selected from SEQ ID NOs: 73-77.

In some embodiments, the ECI comprises an amino acid sequence that is at least 70% identical to an amino acid sequence selected from SEQ ID NO: 30, SEQ ID NO: 34; and SEQ ID NO: 35. In some embodiments, the ECI comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NO: 30, SEQ ID NO: 34; and SEQ ID NO: 35. In some embodiments, the ECI comprises an amino acid sequence having from 1 to 20, from 1 to 10, or from 1 to 5 amino acid modifications independently selected from substitutions, insertions, and deletions with respect to an amino acid sequence selected from SEQ ID NO: 30, SEQ ID NO: 34; and SEQ ID NO: 35.

In some embodiments, the ECI comprises an amino acid sequence that is at least 70% identical to the amino acid sequence of SEQ ID NO: 30. In some embodiments, the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 30. In some embodiments, the ECI comprises an amino acid sequence having from 1 to 20, from 1 to 10, or from 1 to 5 amino acid modifications independently selected from substitutions, insertions, and deletions with respect to the amino acid sequence of SEQ ID NO: 30.

In some embodiments, the ECI comprises an amino acid sequence that is at least 70% identical to the amino acid sequence of SEQ ID NO: 34. In some embodiments, the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 34. In some embodiments, the ECI comprises an amino acid sequence having from 1 to 20, from 1 to 10, or from 1 to 5 amino acid modifications independently selected from substitutions, insertions, and deletions with respect to the amino acid sequence of SEQ ID NO: 34.

In some embodiments, the ECI comprises an amino acid sequence that is at least 70% identical to the amino acid sequence of SEQ ID NO: 35. In some embodiments, the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 35. In some embodiments, the ECI comprises an amino acid sequence having from 1 to 20, from 1 to 10, or from 1 to 5 amino acid modifications independently selected from substitutions, insertions, and deletions with respect to the amino acid sequence of SEQ ID NO: 35.

In some embodiments, the ECI comprises an amino acid sequence that is at least 70% identical to the amino acid sequence of SEQ ID NO: 21. In some embodiments, the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 21. In some embodiments, the ECI comprises an amino acid sequence having from 1 to 20, from 1 to 10, or from 1 to 5 amino acid modifications independently selected from substitutions, insertions, and deletions with respect to the amino acid sequence of SEQ ID NO: 21.

In some embodiments, the ECI comprises an amino acid sequence that is at least 70% identical to the amino acid sequence of SEQ ID NO: 73. In some embodiments, the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 73. In some embodiments, the ECI comprises an amino acid sequence having from 1 to 20, from 1 to 10, or from 1 to 5 amino acid modifications independently selected from substitutions, insertions, and deletions with respect to the amino acid sequence of SEQ ID NO: 73.

In one aspect, the present disclosure relates to a microbial host cell expressing a recombinant biosynthetic pathway comprising a fatty acyl-CoA reductase (FAR), wherein the microbial cell and/or the FAR is capable of converting cis-3-hexenoyl-CoA to cis-3-hexenal. Any FAR that is capable of converting cis-3-hexenoyl-CoA to cis-3-hexenal can be employed in various embodiments. In some embodiments, the FAR uses NAD and/or NADPH as a cofactor. In some embodiments, the FAR uses NADPH as a cofactor.

In some embodiments, the FAR comprises an amino acid sequence that is at least 70% identical to an amino acid sequence selected from SEQ ID NOs: 36-46 and 52-72. In some embodiments, the FAR comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 36-46 and 52-72. In some embodiments, the FAR comprises an amino acid sequence having from 1 to 20, from 1 to 10, or from 1 to 5 amino acid modifications independently selected from substitutions, insertions, and deletions with respect to an amino acid sequence selected from SEQ ID NOs: 36-46 and 52-72.

In some embodiments, the microbial host cell is a yeast (without limitation, e.g., Yarrowia lipolytica), and the FAR comprises an amino acid sequence that is at least 70% identical to an amino acid sequence selected from SEQ ID NOs: 36-46. In some embodiments, the microbial host cell is a yeast (without limitation, e.g., Yarrowia lipolytica), and the FAR comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 36-46. In some embodiments, the microbial host cell is a yeast (without limitation, e.g., Yarrowia lipolytica), and the FAR comprises an amino acid sequence having from 1 to 20, from 1 to 10, or from 1 to 5 amino acid modifications independently selected from substitutions, insertions, and deletions with respect to an amino acid sequence selected from SEQ ID NOs: 36-46.

In some embodiments, the microbial host cell is a bacterium (without limitation, e.g., Escherichia coli), and the FAR comprises an amino acid sequence that is at least 70% identical to an amino acid sequence selected from SEQ ID NOs: 52-72. In some embodiments, the microbial host cell is a bacterium (without limitation, e.g., Escherichia coli), and the FAR comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 52-72. In some embodiments, the microbial host cell is a bacterium (without limitation, e.g., Escherichia coli), and the FAR comprises an amino acid sequence having from 1 to 20, from 1 to 10, or from 1 to 5 amino acid modifications independently selected from substitutions, insertions, and deletions with respect to an amino acid sequence selected from SEQ ID NOs: 52-72.

In some embodiments, the FAR comprises an amino acid sequence that is at least 70% identical to an amino acid sequence selected from SEQ ID NO: 36, SEQ ID NO: 37, and SEQ ID NO: 38. In some embodiments, the FAR comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NO: 36, SEQ ID NO: 37, and SEQ ID NO: 38. In some embodiments, the FAR comprises an amino acid sequence having from 1 to 20, from 1 to 10, or from 1 to 5 amino acid modifications independently selected from substitutions, insertions, and deletions with respect to an amino acid sequence selected from SEQ ID NO: 36, SEQ ID NO: 37, and SEQ ID NO: 38.

In some embodiments, the FAR comprises an amino acid sequence that is at least 70% identical to the amino acid sequence of SEQ ID NO: 36. In some embodiments, the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 36. In some embodiments, the FAR comprises an amino acid sequence having from 1 to 20, from 1 to 10, or from 1 to 5 amino acid modifications independently selected from substitutions, insertions, and deletions with respect to the amino acid sequence of SEQ ID NO: 36.

In some embodiments, the FAR comprises an amino acid sequence that is at least 70% identical to the amino acid sequence of SEQ ID NO: 37. In some embodiments, the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 37. In some embodiments, the FAR comprises an amino acid sequence having from 1 to 20, from 1 to 10, or from 1 to 5 amino acid modifications independently selected from substitutions, insertions, and deletions with respect to the amino acid sequence of SEQ ID NO: 37.

In some embodiments, the FAR comprises an amino acid sequence that is at least 70% identical to the amino acid sequence of SEQ ID NO: 38. In some embodiments, the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 38. In some embodiments, the FAR comprises an amino acid sequence having from 1 to 20, from 1 to 10, or from 1 to 5 amino acid modifications independently selected from substitutions, insertions, and deletions with respect to the amino acid sequence of SEQ ID NO: 38.

In some embodiments, the FAR comprises an amino acid sequence that is at least 70% identical to the amino acid sequence of SEQ ID NO: 58. In some embodiments, the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 58. In some embodiments, the FAR comprises an amino acid sequence having from 1 to 20, from 1 to 10, or from 1 to 5 amino acid modifications independently selected from substitutions, insertions, and deletions with respect to the amino acid sequence of SEQ ID NO: 58.

In some embodiments, the FAR comprises an amino acid sequence that is at least 70% identical to the amino acid sequence of SEQ ID NO: 59. In some embodiments, the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 59. In some embodiments, the FAR comprises an amino acid sequence having from 1 to 20, from 1 to 10, or from 1 to 5 amino acid modifications independently selected from substitutions, insertions, and deletions with respect to the amino acid sequence of SEQ ID NO: 59.

In some embodiments, the FAR comprises an amino acid sequence that is at least 70% identical to the amino acid sequence of SEQ ID NO: 64. In some embodiments, the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 64. In some embodiments, the FAR comprises an amino acid sequence having from 1 to 20, from 1 to 10, or from 1 to 5 amino acid modifications independently selected from substitutions, insertions, and deletions with respect to the amino acid sequence of SEQ ID NO: 64.

In some embodiments, the FAR comprises an amino acid sequence that is at least 70% identical to the amino acid sequence of SEQ ID NO: 65. In some embodiments, the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 65. In some embodiments, the FAR comprises an amino acid sequence having from 1 to 20, from 1 to 10, or from 1 to 5 amino acid modifications independently selected from substitutions, insertions, and deletions with respect to the amino acid sequence of SEQ ID NO: 65.

In one aspect, the present disclosure relates to a microbial host cell expressing a recombinant biosynthetic pathway comprising an alcohol dehydrogenase (ADH), wherein the microbial cell and/or the ADH is capable of converting the cis-3-hexenal to cis-3-hexenol. Any ADH that is capable of converting the cis-3-hexenal to cis-3-hexenol can be employed in various embodiments. In some embodiments, the ADH uses NADH and/or NADPH as a cofactor. In some embodiments, the ADH uses NADPH as a cofactor.

In some embodiments, the ADH comprises an amino acid sequence that is at least 70% identical to an amino acid sequence selected from SEQ ID NOs: 47-51 and 83-88. In some embodiments, the ADH comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 47-51 and 83-88. In some embodiments, the ADH comprises an amino acid sequence having from 1 to 20, from 1 to 10, or from 1 to 5 amino acid modifications independently selected from substitutions, insertions, and deletions with respect to an amino acid sequence selected from SEQ ID NOs: 47-51 and 83-88.

In some embodiments, the microbial host cell is a yeast (without limitation, e.g., Yarrowia lipolytica), and the ADH comprises an amino acid sequence that is at least 70% identical to an amino acid sequence selected from SEQ ID NOs: 47-51. In some embodiments, the microbial host cell is a yeast (without limitation, e.g., Yarrowia lipolytica), and the ADH comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 47-51. In some embodiments, the microbial host cell is a yeast (without limitation, e.g., Yarrowia lipolytica), and the ADH comprises an amino acid sequence having from 1 to 20, from 1 to 10, or from 1 to 5 amino acid modifications independently selected from substitutions, insertions, and deletions with respect to an amino acid sequence selected from SEQ ID NOs: 47-51.

In some embodiments, the microbial host cell is a bacterium (without limitation, e.g., Escherichia coli), and the ADH comprises an amino acid sequence that is at least 70% identical to an amino acid sequence selected from SEQ ID NOs: 83-88. In some embodiments, the microbial host cell is a bacterium (without limitation, e.g., Escherichia coli), and the ADH comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 83-88. In some embodiments, the microbial host cell is a bacterium (without limitation, e.g., Escherichia coli), and the ADH comprises an amino acid sequence having from 1 to 20, from 1 to 10, or from 1 to 5 amino acid modifications independently selected from substitutions, insertions, and deletions with respect to an amino acid sequence selected from SEQ ID NOs: 83-88.

In some embodiments, the ADH comprises an amino acid sequence that is at least 70% identical to the amino acid sequence of SEQ ID NO: 47. In some embodiments, the ADH comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 47. In some embodiments, the ADH comprises an amino acid sequence having from 1 to 20, from 1 to 10, or from 1 to 5 amino acid modifications independently selected from substitutions, insertions, and deletions with respect to the amino acid sequence of SEQ ID NO: 47.

In some embodiments, the ADH comprises an amino acid sequence that is at least 70% identical to the amino acid sequence of SEQ ID NO: 83. In some embodiments, the ADH comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 83. In some embodiments, the ADH comprises an amino acid sequence having from 1 to 20, from 1 to 10, or from 1 to 5 amino acid modifications independently selected from substitutions, insertions, and deletions with respect to the amino acid sequence of SEQ ID NO: 83.

In some embodiments, the ADH comprises an amino acid sequence that is at least 70% identical to the amino acid sequence of SEQ ID NO: 84. In some embodiments, the ADH comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 84. In some embodiments, the ADH comprises an amino acid sequence having from 1 to 20, from 1 to 10, or from 1 to 5 amino acid modifications independently selected from substitutions, insertions, and deletions with respect to the amino acid sequence of SEQ ID NO: 84.

In some embodiments, the ADH comprises an amino acid sequence that is at least 70% identical to the amino acid sequence of SEQ ID NO: 87. In some embodiments, the ADH comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 87. In some embodiments, the ADH comprises an amino acid sequence having from 1 to 20, from 1 to 10, or from 1 to 5 amino acid modifications independently selected from substitutions, insertions, and deletions with respect to the amino acid sequence of SEQ ID NO: 87.

In some embodiments, the microbial host cell capable of producing cis-3-hexenol is a yeast (without limitation, e.g., Yarrowia lipolytica) and the microbial cell expresses a recombinant biosynthetic pathway comprising: (a) an acyl-CoA synthetase (ACS) converting hexanoic acid to hexanoyl-CoA; (b) a short chain acyl-CoA oxidase (AOX) converting the hexanoyl-CoA to trans-2-hexenoyl-CoA; (c) optionally, an enoyl-CoA isomerase (ECI) converting the trans-2-hexenoyl-CoA to cis-3-hexenoyl-CoA; (d) a fatty acyl-CoA reductase (FAR) converting the cis-3-hexenoyl-CoA to cis-3-hexenal; and (e) optionally, an alcohol dehydrogenase (ADH) converting the cis-3-hexenal to cis-3-hexenol. In some embodiments, the recombinant biosynthetic pathway comprises an ECI and/or an ADH. In some embodiments, the enzymatic conversions of (c) and/or (d) are conducted wholly or partly by native enzymes of the host cell. In some embodiments, the yeast (without limitation, e.g., Yarrowia lipolytica) expresses an ACS comprising an amino acid sequence that is at least 70% identical to an amino acid sequence selected from SEQ ID NOs: 1-7. In some embodiments, the yeast (without limitation, e.g., Yarrowia lipolytica) expresses an ACS comprising an amino acid sequence that is at least 70% identical to the amino acid sequence 70% identical to an amino acid sequence selected from SEQ ID NO: 2, SEQ ID NO: 3 and SEQ ID NO: 6. In some embodiments, the yeast (without limitation, e.g., Yarrowia lipolytica) expresses an AOX comprising an amino acid sequence that is at least 70% identical to an amino acid sequence selected from SEQ ID NOs: 8-20. In some embodiments, the yeast (without limitation, e.g., Yarrowia lipolytica) expresses an AOX comprising an amino acid sequence that is at least 70% identical to the amino acid sequence 70% identical to an amino acid sequence selected from SEQ ID NO: 11, SEQ ID NO: 12 and SEQ ID NO: 14. In some embodiments, the yeast (without limitation, e.g., Yarrowia lipolytica) expresses an ECI comprising an amino acid sequence that is at least 70% identical to an amino acid sequence selected from SEQ ID NOs: 21-35. In some embodiments, the yeast (without limitation, e.g., Yarrowia lipolytica) expresses an ECI comprising an amino acid sequence that is at least 70% identical to the amino acid sequence 70% identical to an amino acid sequence selected from SEQ ID NO: 30, SEQ ID NO: 34 and SEQ ID NO: 35. In some embodiments, the yeast (without limitation, e.g., Yarrowia lipolytica) expresses a FAR comprising an amino acid sequence that is at least 70% identical to an amino acid sequence selected from SEQ ID NOs: 36-46. In some embodiments, the yeast (without limitation, e.g., Yarrowia lipolytica) expresses a FAR comprising an amino acid sequence that is at least 70% identical to the amino acid sequence 70% identical to an amino acid sequence selected from SEQ ID NO: 36, SEQ ID NO: 37 and SEQ ID NO: 38. In some embodiments, the yeast (without limitation, e.g., Yarrowia lipolytica) expresses an ADH comprising an amino acid sequence that is at least 70% identical to an amino acid sequence selected from SEQ ID NOs: 47-51. In some embodiments, the yeast (without limitation, e.g., Yarrowia lipolytica) expresses an ADH comprising an amino acid sequence that is at least 70% identical to the amino acid sequence 70% identical to an amino acid sequence of SEQ ID NO: 47. In some embodiments, the yeast (without limitation, e.g., Yarrowia lipolytica) expresses, a heterologous metabolic pathway was constructed using the ACS2 (SEQ ID NO: 2), AOX5 (SEQ ID NO: 12), ECI15 (SEQ ID NO: 35), FAR1 (SEQ ID NO: 36), and ADH1 (SEQ ID NO: 47).

In some embodiments, the microbial host cell capable of producing cis-3-hexenol is a bacterium (without limitation, e.g., Escherichia coli)) and the microbial cell expresses a recombinant biosynthetic pathway comprising: (a) an acyl-CoA synthetase (ACS) converting hexanoic acid to hexanoyl-CoA; (b) a short chain acyl-CoA oxidase (AOX) converting the hexanoyl-CoA to trans-2-hexenoyl-CoA; (c) optionally. an enoyl-CoA isomerase (ECI) converting the trans-2-hexenoyl-CoA to cis-3-hexenoyl-CoA; (d) a fatty acyl-CoA reductase (FAR) converting the cis-3-hexenoyl-CoA to cis-3-hexenal; and (e), optionally, an alcohol dehydrogenase (ADH) converting the cis-3-hexenal to cis-3-hexenol. In some embodiments, the recombinant biosynthetic pathway comprises an ECI and/or an ADH. In some embodiments, the enzymatic conversions of (c) and (d) are conducted wholly or partly by native enzymes of the host cell. In some embodiments, the bacterium (without limitation, e.g., Escherichia coli)) expresses an ACS comprising an amino acid sequence that is at least 70% identical to an amino acid sequence selected from SEQ ID NOs: 1-7. In some embodiments, the bacterium (without limitation, e.g., Escherichia coli)) expresses an ACS comprising an amino acid sequence that is at least 70% identical to the amino acid sequence 70% identical to an amino acid sequence selected from SEQ ID NO: 2, SEQ ID NO: 3 and SEQ ID NO: 6. In some embodiments, the bacterium (without limitation, e.g., Escherichia coli)) expresses an AOX comprising an amino acid sequence that is at least 70% identical to an amino acid sequence selected from SEQ ID NOs: 8-20. In some embodiments, the bacterium (without limitation, e.g., Escherichia coli)) expresses an AOX comprising an amino acid sequence that is at least 70% identical to the amino acid sequence 70% identical to an amino acid sequence selected from SEQ ID NO: 11, SEQ ID NO: 12 and SEQ ID NO: 14. In some embodiments, the bacterium (without limitation, e.g., Escherichia coli)) expresses an ECI comprising an amino acid sequence that is at least 70% identical to an amino acid sequence selected from SEQ ID NOs: 21 and 73-77. In some embodiments, the bacterium (without limitation, e.g., Escherichia coli)) expresses an ECI comprising an amino acid sequence that is at least 70% identical to the amino acid sequence 70% identical to an amino acid sequence selected from SEQ ID NO: 21 and SEQ ID NO: 73. In some embodiments, the bacterium (without limitation, e.g., Escherichia coli)) expresses a FAR comprising an amino acid sequence that is at least 70% identical to an amino acid sequence selected from SEQ ID NOs: 52-72. In some embodiments, the bacterium (without limitation, e.g., Escherichia coli)) expresses a FAR comprising an amino acid sequence that is at least 70% identical to the amino acid sequence 70% identical to an amino acid sequence selected from SEQ ID NO: 58, SEQ ID NO: 59, SEQ ID NO: 64, and SEQ ID NO: 65. In some embodiments, the bacterium (without limitation, e.g., Escherichia coli)) expresses an ADH comprising an amino acid sequence that is at least 70% identical to an amino acid sequence selected from SEQ ID NOs: 83-88. In some embodiments, the bacterium (without limitation, e.g., Escherichia coli)) expresses an ADH comprising an amino acid sequence that is at least 70% identical to the amino acid sequence 70% identical to an amino acid sequence selected from SEQ ID NO: 83, SEQ ID NO: 84, and SEQ ID NO: 87. In some embodiments, the bacterium (without limitation, e.g., Escherichia coli)) expresses, a heterologous metabolic pathway was constructed using the ACS2 (SEQ ID NO: 2), AOX5 (SEQ ID NO: 12), ECI15 (SEQ ID NO: 35), FAR18/FAR19/FAR24/FAR25 (SEQ ID NOs: 58, 59, 64 and 65), and ADH6/ADH7/ADH10, (SEQ ID NO: 83, SEQ ID NO: 84 and SEQ ID NO: 87).

In one aspect, the present disclosure relates to a microbial host cell producing cis-3-hexenol from hexanoic acid. In some embodiments, hexanoic acid is provided externally to the culture. In some embodiments, the microbial host cell converts the externally provided hexanoic acid to hexanoyl-CoA; hexanoyl-CoA to trans-2-hexenoyl-CoA; trans-2-hexenoyl-CoA to cis-3-hexenoyl-CoA; cis-3-hexenoyl-CoA to cis-3-hexenal; and cis-3-hexenal to cis-3-hexenol. In alternative embodiments, hexanoic acid is generated from the intermediates of glycolysis and/or the pentose phosphate pathway. In some embodiments, microbial host cell producing cis-3-hexenol from hexanoic acid produces hexanoic acid that it converts to cis-3-hexenol via the intermediates trans-2-hexenoyl-CoA, cis-3-hexenoyl-CoA, and cis-3-hexenal. In some embodiments, microbial host cell producing hexanoic acid expresses biosynthetic pathway that produces hexanoic acid from intermediates of glycolysis and/or the pentose phosphate pathway.

Accordingly, in some embodiments, the microbial strain expresses an acyl-acyl carrier protein (ACP) thioesterase (TES). In some embodiments, the TES is capable of producing fatty acids and beta-keto fatty acids from a fatty acyl-ACP complex. In some embodiments, the TES, when expressed in a heterologous host is capable of producing fatty acids and beta-keto fatty acids from a fatty acyl-ACP complex. In some embodiments, the fatty acyl-ACP complex comprises medium to long chain (6:0, 8:0, 10:0 and 16:1) fatty acids. In some embodiments, the fatty acyl-ACP complex comprises medium to long chain beta-keto fatty acids (8:0, 14:0 and 16:1). In some embodiments, the microbial host cell expressing a TES is a bacterium (without limitation, e.g., Escherichia coli).

In some embodiments, the TES comprises an amino acid sequence that is at least 70% identical to an amino acid sequence selected from SEQ ID NOs: 78-82. In some embodiments, the TES comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 78-82. In some embodiments, the TES comprises an amino acid sequence having from 1 to 20, from 1 to 10, or from 1 to 5 amino acid modifications independently selected from substitutions, insertions, and deletions with respect to an amino acid sequence selected from SEQ ID NOs: 78-82.

In some embodiments, the TES comprises an amino acid sequence that is at least 70% identical to the amino acid sequence of SEQ ID NO: 79. In some embodiments, the TES comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 79. In some embodiments, the TES comprises an amino acid sequence having from 1 to 20, from 1 to 10, or from 1 to 5 amino acid modifications independently selected from substitutions, insertions, and deletions with respect to the amino acid sequence of SEQ ID NO: 79.

In some embodiments, the TES comprises an amino acid sequence that is at least 70% identical to the amino acid sequence of SEQ ID NO: 81. In some embodiments, the TES comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 081. In some embodiments, the TES comprises an amino acid sequence having from 1 to 20, from 1 to 10, or from 1 to 5 amino acid modifications independently selected from substitutions, insertions, and deletions with respect to the amino acid sequence of SEQ ID NO: 81.

In some embodiments, the microbial strain expresses an enoyl-acyl-carrier-protein (ACP) reductase (ENR). In some embodiments, the ENR is capable of reducing a carbon-carbon double bond in an enoyl moiety that is covalently linked to an acyl carrier protein (ACP). In some embodiments, the ENR, when expressed in a heterologous host is capable of reducing a carbon-carbon double bond in an enoyl moiety that is covalently linked to an acyl carrier protein (ACP). In some embodiments, the fatty acyl-ACP complex comprises medium to long chain (6:0, 8:0, 10:0 and 16:1) fatty acids. In some embodiments, the ENR uses NADH and/or NADPH as a cofactor. In some embodiments, the ENR uses NADPH as a cofactor. In some embodiments, the microbial host cell expressing a ENR is a bacterium (without limitation, e.g., Escherichia coli).

In some embodiments, the ENR comprises an amino acid sequence that is at least 70% identical to an amino acid sequence of SEQ ID NO: 89. In some embodiments, the ENR comprises an amino acid sequence that is at least about 80%, or at least about 85% or at least about 90% or at least about 95%, at least about 96%, at least about 97%, at least about 98%, or at least about 99% identical to an amino acid sequence of SEQ ID NO: 89. In some embodiments, the ENR comprises an amino acid sequence having from 1 to 20, from 1 to 10, or from 1 to 5 amino acid modifications independently selected from substitutions, insertions, and deletions with respect to an amino acid sequence of SEQ ID NO: 89.

In some embodiments, the microbial host cell (without limitation, e.g., a bacterium such as Escherichia coli) has a modification that causes a decreased expression or activity of a multifunctional enzyme that is involved in the degradation of fatty acids via the β-oxidation cycle. In some embodiments, the multifunctional enzyme that is involved in the degradation of fatty acids via the β-oxidation cycle is encoded by fadB, or an ortholog, an analog, or homolog thereof. In some embodiments, the microbial host cell (without limitation, e.g., a bacterium such as Escherichia coli) has a complete or partial deletion or a null mutation or a hypomorphic mutation in the multifunctional enzyme that is involved in the degradation of fatty acids via the β-oxidation cycle. In some embodiments, the microbial host cell (without limitation, e.g., a bacterium such as Escherichia coli) has a complete or partial deletion or a null mutation or a hypomorphic mutation in fadB.

In some embodiments, the microbial host cell (without limitation, e.g., a bacterium such as Escherichia coli) has a modification that causes a decreased expression or activity of a fatty acid oxidation complex 3-hydroxyacyl-CoA dehydrogenase/enoyl-CoA hydratase/3-hydroxybutyryl-CoA epimerase. In some embodiments, the fatty acid oxidation complex 3-hydroxyacyl-CoA dehydrogenase/enoyl-CoA hydratase/3-hydroxybutyryl-CoA epimerase is encoded by fadJ, or an ortholog, an analog, or homolog thereof. In some embodiments, the microbial host cell (without limitation, e.g., a bacterium such as Escherichia coli) has a complete or partial deletion or a null mutation or a hypomorphic mutation in the fatty acid oxidation complex 3-hydroxyacyl-CoA dehydrogenase/enoyl-CoA hydratase/3-hydroxybutyryl-CoA epimerase. In some embodiments, the microbial host cell (without limitation, e.g., a bacterium such as Escherichia coli) has a complete or partial deletion or a null mutation or a hypomorphic mutation in fadJ.

In some embodiments, the microbial host cell (without limitation, e.g., a bacterium such as Escherichia coli) has a modification that causes a decreased expression or activity of a long chain fatty acid CoA-ligase, also known as acyl-CoA synthetase or synthase. In some embodiments, the long chain fatty acid CoA-ligase, also known as acyl-CoA synthetase or synthase is encoded by fadD, or an ortholog, an analog, or homolog thereof. In some embodiments, the microbial host cell (without limitation, e.g., a bacterium such as Escherichia coli) has a complete or partial deletion or a null mutation or a hypomorphic mutation in the long chain fatty acid CoA-ligase, also known as acyl-CoA synthetase or synthase. In some embodiments, the microbial host cell (without limitation, e.g., a bacterium such as Escherichia coli) has a complete or partial deletion or a null mutation or a hypomorphic mutation in fadD.

In some embodiments, the microbial host cell (without limitation, e.g., a bacterium such as Escherichia coli) has a modification that causes a decreased expression or activity of a 3-ketoacyl-CoA thiolase, also known as acyl-CoA synthetase or synthase. In some embodiments, the 3-ketoacyl-CoA thiolase, also known as acyl-CoA synthetase or synthase is encoded by fadI, or an ortholog, an analog, or homolog thereof. In some embodiments, the microbial host cell (without limitation, e.g., a bacterium such as Escherichia coli) has a complete or partial deletion or a null mutation or a hypomorphic mutation in the 3-ketoacyl-CoA thiolase, also known as acyl-CoA synthetase or synthase. In some embodiments, the microbial host cell (without limitation, e.g., a bacterium such as Escherichia coli) has a complete or partial deletion or a null mutation or a hypomorphic mutation in fadI.

In some embodiments, the microbial host cell (without limitation, e.g., a bacterium such as Escherichia coli) has a modification that causes a decreased expression or activity of a oxepin-CoA hydrolase/3-oxo-5,6-dehydrosuberyl-CoA semialdehyde dehydrogenase. In some embodiments, the oxepin-CoA hydrolase/3-oxo-5,6-dehydrosuberyl-CoA semialdehyde dehydrogenase is encoded by paaZ, or an ortholog, an analog, or homolog thereof. In some embodiments, the microbial host cell (without limitation, e.g., a bacterium such as Escherichia coli) has a complete or partial deletion or a null mutation or a hypomorphic mutation in the oxepin-CoA hydrolase/3-oxo-5,6-dehydrosuberyl-CoA semialdehyde dehydrogenase. In some embodiments, the microbial host cell (without limitation, e.g., a bacterium such as Escherichia coli) has a complete or partial deletion or a null mutation or a hypomorphic mutation in paaZ.

In some embodiments, the strain has increased expression or activity of one or more catalase enzymes. In some embodiments, the catalase is a cytosolic catalase. In some embodiments, the catalase is a peroxisomal catalase. In some embodiments, the catalase is active in the cellular compartment where the AOX produces hydrogen peroxide as a byproduct.

In one aspect, the present disclosure relates to a microbial host cell (and methods) producing compounds structurally related to cis-3-hexenol, and which are or can be produced from products and intermediates of the pathway shown in FIG. 1. Enzymes disclosed herein can be used to construct such recombinant biosynthetic pathways for expression in host cells. Such compounds include, but are not limited to, cis-3-hexenoic acid, cis-3-hexenal, cis-3-hexenoyl acetate, cis-3-hexenoyl propionate, cis-3-hexenoyl formate, cis-3-hexenoyl butyrate, cis-3-hexenoyl hexanoate, cis-3-hexenoyl cis-3-hexenoate, cis-3-hexenoyl lactate, cis-3-hexenoyl acetoacetate, trans-2-hexenal, trans-2-hexenol, trans-2-hexenoic acid, trans-2-hexenoyl propionate, trans-2-hexenoyl hexenoate, and ethyl trans-2-hexenoate. In some embodiments, the biosynthetic pathway produces in intermediate shown in FIG. 1, such as cis-3-hexenal or a derivative thereof such as cis-3-hexenoic acid, or a cis-3-hexenoyl ester. In some embodiments, the biosynthetic pathway produces a derivative of trans-2-hexenoyl-CoA, such as trans-2-hexenal, trans-2-hexenol, trans-2-hexenoic acid, or a trans-2-hexenoyl ester.

In exemplary embodiments, the microbial cell produces cis-3-hexenal or a derivative thereof, and expresses a recombinant biosynthetic pathway comprising: (a) an acyl-CoA synthetase (ACS) converting hexanoic acid to hexanoyl-CoA; (b) a short chain acyl-CoA oxidase (AOX) converting the hexanoyl-CoA to trans-2-hexenoyl-CoA; and (c) a fatty acyl-CoA reductase (FAR) converting the cis-3-hexenoyl-CoA to cis-3-hexenal. These enzymes are disclosed herein. In some embodiments, the recombinant biosynthetic pathway comprises an enoyl-CoA isomerase (ECI) that is capable of converting the trans-2-hexenoyl-CoA to cis-3-hexenoyl-CoA and/or an alcohol dehydrogenase (ADH) that is capable of converting the cis-3-hexenal to cis-3-hexenol. In some embodiments, the enzymatic conversions of cis-3-hexenal to cis-3-hexenol and/or trans-2-hexenoyl-CoA to cis-3-hexenoyl-CoA are conducted wholly or partly by native enzymes of the host cell. In some embodiments, the cis-3-hexenoyl-CoA is converted enzymatically (e.g., as a component of the biosynthetic pathway) or non-enzymatically to cis-3-hexenoic acid. In some embodiments, the cis-3-hexenal is oxidized enzymatically (e.g., as a component of the biosynthetic pathway) or non-enzymatically to cis-3-hexenoic acid. In some embodiments, the cis-3-hexenoic acid is converted enzymatically (e.g., as a component of the biosynthetic pathway) or non-enzymatically to a cis-3-hexenoyl ester. In some embodiments, the cis-3-hexenoyl ester is selected from cis-3-hexenoyl acetate, cis-3-hexenoyl propionate, cis-3-hexenoyl formate, cis-3-hexenoyl butyrate, cis-3-hexenoyl hexanoate, cis-3-hexenoyl cis-3-hexenoate, cis-3-hexenoyl lactate, and cis-3-hexenoyl acetoacetate.

In exemplary embodiments, the microbial cell produces trans-3-hexenal or a derivative thereof, and expresses a recombinant biosynthetic pathway comprising: (a) an acyl-CoA synthetase (ACS) converting hexanoic acid to hexanoyl-CoA; (b) a short chain acyl-CoA oxidase (AOX) converting the hexanoyl-CoA to trans-2-hexenoyl-CoA; (c) optionally, an enoyl-CoA isomerase (ECI) converting the trans-2-hexenoyl-CoA to trans-3-hexenoyl-CoA; and (d) a fatty acyl-CoA reductase (FAR) converting the trans-3-hexenoyl-CoA to trans-3-hexenal. These enzymes are disclosed herein. In some embodiments, the recombinant biosynthetic pathway comprises an ECI. In some embodiments, the recombinant biosynthetic pathway comprises an ECI and/or an ADH. In some embodiments, the enzymatic conversions of (c) is conducted wholly or partly by native enzymes of the host cell. In some embodiments, the trans-3-hexenoyl-CoA is converted enzymatically (e.g., as a component of the biosynthetic pathway) or non-enzymatically to trans-3-hexenoic acid. In some embodiments, the trans-3-hexenal is oxidized enzymatically (e.g., as a component of the biosynthetic pathway) or non-enzymatically to trans-3-hexenoic acid. In some embodiments, the trans-3-hexenoic acid is converted enzymatically (e.g., as a component of the biosynthetic pathway) or non-enzymatically to a trans-3-hexenoyl ester. In some embodiments, the trans-3-hexenoyl ester is selected from trans-3-hexenoyl acetate, trans-3-hexenoyl propionate, trans-3-hexenoyl formate, trans-3-hexenoyl butyrate, trans-3-hexenoyl hexanoate, trans-3-hexenoyl trans-3-hexenoate, trans-3-hexenoyl lactate, and trans-3-hexenoyl acetoacetate.

In exemplary embodiments, the microbial cell produces trans-2-hexenal or a derivative thereof, and expresses a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA. Such enzymes are described herein. In some embodiments, the trans-2-hexenoyl-CoA is converted enzymatically (e.g., as a component of the biosynthetic pathway) or non-enzymatically to trans-2-hexenoic acid. In some embodiments, the trans-2-hexenoyl-CoA is enzymatically (e.g., as a component of the biosynthetic pathway) or non-enzymatically converted to trans-2-hexenal.

In exemplary embodiments, the microbial cell produces cis-2-hexenal or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA and (c) an enoyl-CoA isomerase (ECI) converting the trans-2-hexenoyl-CoA to cis-2-hexenoyl-CoA. In some embodiments, the cis-2-hexenoyl-CoA is converted enzymatically (e.g., as a component of the biosynthetic pathway) or non-enzymatically to cis-2-hexenoic acid. In some embodiments, the cis-2-hexenoyl-CoA is enzymatically (e.g., as a component of the biosynthetic pathway) or non-enzymatically converted to cis-2-hexenal.

In exemplary embodiments, the microbial cell produces cis-2-hexenol or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA; and (c) an enoyl-CoA isomerase (ECI) converting the trans-2-hexenoyl-CoA to cis-2-hexenoyl-CoA. In some embodiments, the cis-2-hexenoyl-CoA is converted enzymatically (e.g., as a component of the biosynthetic pathway) or non-enzymatically to cis-2-hexenoic acid. In some embodiments, the cis-2-hexenoyl-CoA is enzymatically (e.g., as a component of the biosynthetic pathway) or non-enzymatically converted to cis-2-hexenal. In some embodiments, the cis-2-hexenal is enzymatically (e.g., as a component of the biosynthetic pathway) or non-enzymatically oxidized to cis-2-hexenoic acid. In some embodiments, the cis-2-hexenoic acid is converted enzymatically (e.g., as a component of the biosynthetic pathway) or non-enzymatically to a cis-2-hexenoyl ester. In some embodiments, the cis-2-hexenoyl ester is selected from cis-2-hexenoic acid, cis-2-hexenoyl ester is selected from cis-2-hexenoyl acetate, cis-2-hexenoyl salicylate, cis-2-hexenoyl propionate, cis-2-hexenoyl formate, cis-2-hexenoyl butyrate, cis-2-hexenoyl hexanoate, cis-2-hexenoyl cis-2-hexenoate, cis-2-hexenoyl lactate, and cis-2-hexenoyl acetoacetate

In exemplary embodiments, the microbial cell produces trans-2-hexenol or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA. In some embodiments, the cis-2-hexenoyl-CoA is enzymatically (e.g., as a component of the biosynthetic pathway) or non-enzymatically converted to cis-2-hexenal. In some embodiments, the cis-2-hexenal is enzymatically (e.g., as a component of the biosynthetic pathway) or non-enzymatically oxidized to cis-2-hexenol. In some embodiments, the cis-2-hexenol is converted enzymatically (e.g., as a component of the biosynthetic pathway) or non-enzymatically to a cis-2-hexenoyl ester. In some embodiments, the cis-2-hexenoyl ester is selected from cis-2-hexenoyl ester is selected from cis-2-hexenoyl acetate, cis-2-hexenoyl salicylate, cis-2-hexenoyl propionate, cis-2-hexenoyl formate, cis-2-hexenoyl butyrate, cis-2-hexenoyl hexanoate, cis-2-hexenoyl cis-2-hexenoate, cis-2-hexenoyl lactate, and cis-2-hexenoyl acetoacetate.

In exemplary embodiments, the microbial cell produces trans-3-hexenol or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA; and (c) an enoyl-CoA isomerase (ECI) converting the trans-2-hexenoyl-CoA to trans-3-hexenoyl-CoA. In some embodiments, the trans-3-hexenoyl-CoA is enzymatically (e.g., as a component of the biosynthetic pathway) or non-enzymatically converted to trans-3-hexenal. In some embodiments, the trans-3-hexenal is enzymatically (e.g., as a component of the biosynthetic pathway) or non-enzymatically oxidized to trans-3-hexenol. In some embodiments, the trans-3-hexenol acid is converted enzymatically (e.g., as a component of the biosynthetic pathway) or non-enzymatically to a trans-3-hexenoyl ester. In some embodiments, the trans-3-hexenoyl ester is selected from trans-3-hexenoyl ester is selected from trans-3-hexenoyl acetate, trans-3-hexenoyl salicylate, trans-3-hexenoyl propionate, trans-3-hexenoyl formate, trans-3-hexenoyl butyrate, trans-3-hexenoyl hexanoate, trans-3-hexenoyl trans-3-hexenoate, trans-3-hexenoyl lactate, and trans-3-hexenoyl acetoacetate.

In exemplary embodiments, the microbial cell produces cis-3-hexenol or a derivative thereof, microbial host cell expressing a recombinant biosynthetic pathway comprising: (a) an acyl-CoA synthetase (ACS) converting hexanoic acid to hexanoyl-CoA; (b) a short chain acyl-CoA oxidase (AOX) converting the hexanoyl-CoA to trans-2-hexenoyl-CoA; and (c) an enoyl-CoA isomerase (ECI) converting the trans-2-hexenoyl-CoA to cis-3-hexenoyl-CoA. In some embodiments, the cis-3-hexenoyl-CoA is enzymatically (e.g., as a component of the biosynthetic pathway) or non-enzymatically converted to cis-3-hexenal. In some embodiments, the cis-3-hexenal is enzymatically (e.g., as a component of the biosynthetic pathway) or non-enzymatically oxidized to cis-3-hexenol. In some embodiments, the cis-3-hexenol is converted enzymatically (e.g., as a component of the biosynthetic pathway) or non-enzymatically to a cis-3-hexenoyl ester. In some embodiments, the cis-3-hexenoyl ester is selected from cis-3-hexenoyl ester is selected from cis-3-hexenoyl acetate, cis-3-hexenoyl salicylate, cis-3-hexenoyl propionate, cis-3-hexenoyl formate, cis-3-hexenoyl butyrate, cis-3-hexenoyl hexanoate, cis-3-hexenoyl cis-3-hexenoate, cis-3-hexenoyl lactate, and cis-3-hexenoyl acetoacetate.

In some embodiments, the microbial host cell comprises one or more genetic modifications (relative to a parent strain) that increase the metabolic supply in the microbial cells of NADPH compared to the parental strain, which is the cofactor of the fatty acyl-CoA reductase, aldehyde reductases, and/or alcohol dehydrogenases. In some embodiments, the microbial cell has one or more modifications that increase metabolic NADPH supply. In some embodiments, the microbial host cell has one or more genetic modification(s) that increase metabolic NADPH supply compared to a parental strain through (i) increased glycolytic flux through the oxidative pentose phosphate pathway; (ii) expression of an alternative or exogenous NADPH biosynthesis route; and/or (iii) increased production of NADPH via tricarboxylic acid intermediates.

In some embodiments, the modifications that increase the metabolic supply of NADPH increase the glycolytic flux through the oxidative pentose phosphate pathway. For example, such modifications can comprise a deletion or reduced amount or activity of: (A) glucose-6-phosphate isomerase; and/or (B) phosphofructokinase. Alternatively or in addition, the modifications that result in increased glycolytic flux through the oxidative pentose phosphate pathway may comprise an increase in the amount or activity of: (A) glucose-6-phosphate dehydrogenase; and/or (B) 6-phosphogluconate dehydrogenase. In some embodiments, the modifications that result in increased glycolytic flux through the oxidative pentose phosphate pathway comprise one or more of an overexpression of glucose-6-phosphate dehydrogenase gene (e.g., ZWF1 gene, or a homolog, or an ortholog thereof) and/or 6-phosphogluconate dehydrogenase gene (e.g., GND1 gene, or a homolog, or an ortholog thereof). In some embodiments, the modifications that increase the metabolic supply of NADPH reduce the expression or activity of glucose-6-phosphate isomerase gene (e.g., PGI1 gene, or an ortholog thereof) and/or phosphofructokinase gene (e.g., PFK1 gene, or an ortholog thereof). In some embodiments, the microbial cell belongs to the species Yarrowia lipolytica and harbors a deletion or inactivation of PGI1 and/or PFK1 gene, and/or harbors an overexpression of ZWF1 and/or GND1 (e.g., a gene complementation), or an ortholog or derivative thereof.

In some embodiments, the microbial cell is engineered to increase the metabolic supply of NADPH by expressing or overexpressing an alternative or exogenous NADPH biosynthesis route. In some embodiments, the alternative or exogenous NADPH biosynthesis route comprises bacterial transhydrogenase expression, and/or a NADP-dependent glyceraldehyde-3-phosphate dehydrogenase expression. In some embodiments, the microbial cell expresses a bacterial pntAB and/or bacterial or plant gapN, or a homolog, or an ortholog thereof, or a variant thereof. In some embodiments, the microbial cell expresses a hyperactive variant of bacterial pntAB and/or bacterial or plant gapN, or a homolog, or an ortholog thereof. In some embodiments, the microbial cell belongs to the species Yarrowia lipolytica and it overexpresses bacterial pntAB and/or bacterial or plant gapN gene.

In these or other embodiments, the microbial cell has a genetic modification that results in increased production of NADPH via tricarboxylic acid intermediates. For example, the microbial cell may have an increased expression or activity of a cytosolic NADP (+)-dependent isocitrate dehydrogenase (e.g., IDH or ortholog thereof).

In some embodiments, the modifications downregulate β-oxidation and peroxisome metabolism. In some embodiments, the downregulation of β-oxidation and peroxisome metabolism is caused by a reduction in the amount or activity of: (i) multifunctional β-oxidation enzyme; (ii) peroxisomal membrane E3 ubiquitin ligase; (iii) peroxisomal membrane protein; (iv) one or more peroxisomal acyl-CoA oxidase; (v) peroxisomal adenine nucleotide transporter; (vi) one or more enoyl-CoA hydratase; (vii) one or more 3-hydroxyacyl-CoA dehydratase; (viii) enoyl-CoA hydratase/isomerase; (ix) 3-hydroxyacyl-CoA dehydrogenase; (x) 3-ketoacyl-CoA thiolase; and/or (xi) acyl-CoA dehydrogenase. In some embodiments, the downregulation β-oxidation and peroxisome is caused by a reduction in the amount or activity of a multifunctional β-oxidation enzyme (encoded by MFE1 gene, or a homolog, or an ortholog thereof). In some embodiments, the downregulation β-oxidation and peroxisome is caused by a reduction in the amount or activity of a peroxisomal membrane E3 ubiquitin ligase (e.g., encoded by PEX10 gene, or a homolog, or an ortholog thereof). In some embodiments, the downregulation β-oxidation and peroxisome is caused by a reduction in the amount or activity of a peroxisomal membrane protein (e.g., encoded by PEX11 gene, or a homolog, or an ortholog thereof). In some embodiments, the downregulation β-oxidation and peroxisome is caused by a reduction in the amount or activity of one or more peroxisomal acyl-CoA oxidase enzymes (e.g., encoded by one or more of POX1, POX2, POX3, POX4, POX5, and/or POX6, genes, or homologs, or orthologs thereof). In some embodiments, the downregulation β-oxidation and peroxisome is caused by a reduction in the amount or activity of a peroxisomal adenine nucleotide transporter (e.g., encoded by ANT1 gene, or a homolog, or an ortholog thereof). In some embodiments, the reduction of β-oxidation and peroxisome metabolism is caused by a deletion, inactivation, or downregulation of gene expression of one or more of (e.g., at least 2, 3, or 4 of MFE1, PEX10, PEX11, POX1, POX2, POX3, POX4, POX5, POX6, and/or ANT1 genes, or homologs, or orthologs thereof). In some embodiments, the reduction of β-oxidation and peroxisome metabolism is caused by a hypomorphic mutation or a null mutation (e.g., a deletion) in one or more of (e.g., at least 2, 3, 4 of MFE1, PEX10, PEX11, POX1, POX2, POX3, POX4, POX5, POX6, and/or ANT1 genes, or homologs, or orthologs thereof). In some embodiments, the microbial cell belongs to the species Yarrowia lipolytica and harbors a deletion or inactivation of one or more of MFE1, PEX10, PEX11, POX1, POX2, POX3, POX4, POX5, POX6, and/or ANT1 genes. In some embodiments, the reduction of β-oxidation and peroxisome metabolism is caused by a hypomorphic mutation or a null mutation (e.g., a deletion) in one or more of enoyl-CoA hydratase/isomerase (e.g., at least 2, or 3 of YALI0B10406g, YALI0F22121g and/or YALI0A07733g genes, or homologs, or orthologs thereof). In some embodiments, the microbial cell belongs to the species Yarrowia lipolytica and harbors a deletion or inactivation of one or more of YALI0B10406g, YALI0F22121g and/or YALI0A07733g genes. In some embodiments, the reduction of β-oxidation and peroxisome metabolism is caused by a hypomorphic mutation or a null mutation (e.g., a deletion) in one or more of 3-hydroxyacyl-CoA dehydratase. In some embodiments, the reduction of β-oxidation and peroxisome metabolism is caused by a hypomorphic mutation or a null mutation (e.g., a deletion) in one or more of 3-hydroxyacyl-CoA dehydratase (e.g., at least 2, or 3, or 4, or 5, or all of YALI0A20207g, YALI0109383g, YALI0D00671g, YALI0D09493g, YALI0F28567g, and/or YALI0C08811g genes, or homologs, or orthologs thereof). In some embodiments, the microbial cell belongs to the species Yarrowia lipolytica and harbors a deletion or inactivation of one or more of YALI0A20207g, YALI0D09383g, YALI0100671g, YALI0D09493g, YALI0F28567g, and/or YALI0C08811g genes. In some embodiments, the reduction of β-oxidation and peroxisome metabolism is caused by a hypomorphic mutation or a null mutation (e.g., a deletion) in a 3-hydroxyacyl-CoA dehydrogenase. In some embodiments, the reduction of β-oxidation and peroxisome metabolism is caused by a hypomorphic mutation or a null mutation (e.g., a deletion) in a 3-hydroxyacyl-CoA dehydrogenase (e.g., YALI0C08811g gene, or homologs, or orthologs thereof). In some embodiments, the microbial cell belongs to the species Yarrowia lipolytica and harbors a deletion or inactivation of YALI0C08811g gene. In some embodiments, the reduction of β-oxidation and peroxisome metabolism is caused by a hypomorphic mutation or a null mutation (e.g., a deletion) in a 3-ketoacyl-CoA thiolase. In some embodiments, the reduction of β-oxidation and peroxisome metabolism is caused by a hypomorphic mutation or a null mutation (e.g., a deletion) in a 3-ketoacyl-CoA thiolase (e.g., YALI0E11099g gene, or homologs, or orthologs thereof). In some embodiments, the microbial cell belongs to the species Yarrowia lipolytica and harbors a deletion or inactivation of YALI0E11099g gene. In some embodiments, the reduction of β-oxidation and peroxisome metabolism is caused by a hypomorphic mutation or a null mutation (e.g., a deletion) in a acyl-CoA dehydrogenase. In some embodiments, the reduction of β-oxidation and peroxisome metabolism is caused by a hypomorphic mutation or a null mutation (e.g., a deletion) in a acyl-CoA dehydrogenase (e.g., YALI0D15708g gene, or homologs, or orthologs thereof). In some embodiments, the microbial cell belongs to the species Yarrowia lipolytica and harbors a deletion or inactivation of YALI0D15708g gene.

In some embodiments, the microbial cell is engineered to reduce the amount or activity of one or more non-essential NADPH dependent aldehyde reductases. This modification is designed to decrease the consumption of NADPH, which is the cofactor for the fatty acid hydroxylase. For example, the genome of Yarrowia lipolytica contains the following putative NADPH-dependent reductases: ALR1-12 (encoded by YALI0D07634g, YALI0C13508g, YALI0F18590g, YALI0F09075g, YALI0F09097g, YALI0A15906g, YALI0C20251g, YALI0C06171g, YALI0C02805g, YALI0B15268g, YALI0B01298g, and/or YALI0B07117g See Cheng et al., Identification, characterization of two NADPH-dependent erythrose reductases in the yeast Yarrowia lipolytica and improvement of erythritol productivity using metabolic engineering, Microb Cell Fact. 17: 133 (2018). In some embodiments, the reduction in the amount or activity of one or more non-essential NADPH dependent aldehyde reductases is caused by a hypomorphic mutation, inactivation, or a null mutation (e.g., a deletion) in one or more of YALI0D07634g, YALI0C13508g, YALI0F18590g, YALI0F09075g, YALI0F09097g, YALI0A15906g, YALI0C20251g, YALI0C06171g, YALI0C02805g, YALI0B15268g, YALI0B01298g, and/or YALI0B07117g genes. In some embodiments, the microbial cell belongs to the species Yarrowia lipolytica and harbors a deletion or inactivation of one or more of (e.g., 2, 3, 4, or more of) YALI0D07634g, YALI0C13508g, YALI0F18590g, YALI0F09075g, YALI0F09097g, YALI0A15906g, YALI0C20251g, YALI0C06171g, YALI0C02805g, YALI0B15268g, YALI0B01298g, and/or YALI0B07117g genes.

In some embodiments, the modifications reduce neutral lipid biosynthesis. In some embodiments, the reduction of neutral lipid biosynthesis is caused by a reduction in the amount or activity (e.g., by deletion, inactivation, or decreased expression) of: (i) diacylglycerol acyltransferase enzyme; and/or (ii) acyl-CoA:sterol acyltransferase. In some embodiments, the reduction of neutral lipid biosynthesis is caused by a reduction in the amount or activity (e.g., by deletion, inactivation, or decreased expression) of DGA1, DGA2, and/or LRO1 genes, or homologs, or orthologs thereof. In some embodiments, the reduction of neutral lipid biosynthesis is caused by a reduction in the amount or activity (e.g., by deletion, inactivation, or decreased expression) of acyl-CoA:sterol acyltransferase (e.g., encoded by ARE1 gene, or a homolog, or an ortholog thereof). In some embodiments, the reduction of neutral lipid biosynthesis is caused by a downregulation of one or more of (e.g., 2, 3, or 4 of) DGA1, DGA2, LRO1, and/or ARE1 gene, or a homolog, or an ortholog thereof. In some embodiments, the microbial cell belongs to the species Yarrowia lipolytica and harbors a deletion of one or more of DGA1, DGA2, LRO1, and ARE1 gene.

In some embodiments, the microbial host cell has one or more modifications that reduce the amount or activity of one or more native alcohol dehydrogenases and/or alcohol oxidase. In some embodiments, the reduction in the amount or activity of one or more non-essential alcohol dehydrogenases is caused by a hypomorphic mutation, inactivation, or a null mutation (e.g., a deletion) in one or more of ADH1, ADH2, ADH3, ADH4, ADH5, ADH6, ADH7, and/or FADH genes. In some embodiments, the microbial cell belongs to the species Yarrowia lipolytica and harbors a deletion or inactivation of one or more of (e.g., 2, 3, 4, or more of) ADH1, ADH2, ADH3, ADH4, ADH5, ADH6, ADH7, and/or FADH genes. In some embodiments, the reduction in the amount or activity of alcohol oxidase is caused by a hypomorphic mutation, inactivation, or a null mutation (e.g., a deletion) in FAO1 gene. In some embodiments, the microbial cell belongs to the species Yarrowia lipolytica and harbors a deletion or inactivation of FAO1 gene.

The similarity or identity of nucleotide and amino acid sequences, i.e. the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215:403-410. BLAST polynucleotide searches can be performed with the BLASTN program, score=100, word length=12.

BLAST protein searches may be performed with the BLASTP program, score=50, word length=3. To obtain gapped alignments for comparative purposes, Gapped BLAST is utilized as described in Altschul et al (1997) Nucleic Acids Res. 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

Expression of enzymes can be tuned for optimal activity, using, for example, gene modules (e.g., operons) or independent expression of the enzymes. For example, expression of the genes can be regulated through selection of promoters, such as inducible or constitutive promoters, with different strengths (e.g., strong, intermediate, or weak). Additionally, expression of genes can be regulated through manipulation of the copy number of the gene in the cell. In some embodiments, expression of genes can be regulated through manipulating the order of the genes within a module, where the genes transcribed first in an operon are generally expressed at a higher level. In some embodiments, expression of genes is regulated through integration of one or more genes into the chromosome.

Optimization of expression can also be achieved through selection of appropriate promoters and ribosomal binding sites. In some embodiments, this may include the selection of high-copy number plasmids, or single-, low- or medium-copy number plasmids. The step of transcription termination can also be targeted for regulation of gene expression, through the introduction or elimination of structures such as stem-loops.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA. The heterologous DNA is placed under operable control of transcriptional elements to permit the expression of the heterologous DNA in the host cell.

In some embodiments, endogenous genes are edited, as opposed to gene complementation. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced by homologous recombination.

In some embodiments, genes are overexpressed at least in part by controlling gene copy number. While gene copy number can be conveniently controlled using plasmids with varying copy number, gene duplication and chromosomal integration can also be employed. For example, a process for genetically stable tandem gene duplication is described in US 2011/0236927, which is hereby incorporated by reference in its entirety.

In accordance with this disclosure, where genes are deleted, genes can be deleted in whole or in part (i.e., inactivated), which can include deletion of coding sequences and/or expression control sequences.

In some embodiments, the microbial cell is a yeast or fungal cell. In some embodiments, the microbial cell is an oleaginous yeast (without limitation, e.g., Yarrowia lipolytica). In some embodiments, the yeast or fungal cell belongs to a genus selected from Ashbya, Aspergillus, Aurantiochytrium, Bastobotyrs, Candida, Claviceps, Cryptococcus, Cunninghamella, Geotrichum, Hansemila, Issatchenkia, Kluyveromyces, Kodamaea, Leucosporidiella, Linderna, Lipomyces, Mortierella, Myxozyma, Mucor, Occultifur, Ogataea, Penicillium, Phaffia, Pichia, Prototheca, Rhizopus, Rhodosporidium, Rhodotorula, Saccharomyces, Scheffersomyces, Schizosaccharomyces, Sporidiobolus, Sporobolomyces, Starmerella, Tremella, Trichosporon, Wickerhamomyces, Waltomyces, and Yarrowia. In some embodiments, the yeast or fungal cell belongs to a species selected from Yarrowia lipolytica, Yarrowia phangngensis, Pichia kudriavzevii, Saccharomyces cerevisiae, Pichia pastoris, Kluyveromyces marxianus, Rhodosporidium toruloides, Sporidiobolus ruinenii, Sporidiobolus salmonicolor, Aspergillus oryzae, Mortierella isabellina, Waltomyces lipofer, Candida tropicalis, Candida boidinii, Scheffersomyces stipitis, Mucor circinelloides, Ashbya gossypii, Trichoderma harzianum, Pichia guilliermondii, Kodamaea ohmeri, Rhodotorula aurantiaca, Lindnera saturnus, Penicillium roqueforti, Lipomyces starkeyi, and Bastobotyrs adeninivorans. In some embodiments, the yeast or fungal cell is Yarrowia lipolytica. In some embodiments, the yeast or fungal cell is Yarrowia phangngensis.

In some embodiments, the microbial cell is a bacterial cell. In some embodiments, the microbial cell is a bacterium that accumulates significant quantities of triacylglycerols (TAGs; without limitation, e.g., Rhodococcus opacus, Acinetobacter calcoaceticus, Streptomyces coelicolor, Rhodococcus jostii, and Acinetobacter baylyi). In some embodiments, the bacterial cell belongs to a genus selected from Acidovorax, Acinetobacter, Actinomyces, Alcanivorax, Arthrobacter, Brevibacterium, Bacillus, Clostridium, Corynebacterium, Dietzia, Escherichia, Gordonia, Marinobacter, Mycobacterium, Micrococcus, Micromonospora, Moraxella, Nocardia, Pseudomonas, Psychrobacter, Rhodococcus, Salmonella, Streptomyces, Thalassolituus, and Thermomonospora. In some embodiments, the bacterial cell belongs to a species selected from Rhodococcus opacus, Acinetobacter calcoaceticus, Streptomyces coelicolor, Rhodococcus jostii, and Acinetobacter baylyi. In some embodiments, the bacterial host cell is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the bacterial host cell is E. coli.

In some embodiments, the host cell is Yarrowia lipolytica having one or more genetic modifications increasing the availability of NADPH described herein. To improve availability of NADPH for hydroxylation of fatty acid, a series of gene knock-outs and gene insertions can be introduced to increase the availability of NADPH. For example, genetic modifications can increase glycolytic flux through the oxidative pentose phosphate pathway, express an alternative or exogenous NADPH biosynthesis route; and/or increase production of NADPH via tricarboxylic acid intermediates. Another series of knock-outs can reduce the utilization of NADPH in other non-essential pathways.

The WT enzymes disclosed herein, can optionally contain an Ala at position 2 (e.g., Ala insertion at position 2) where not present in the wild-type. In some embodiments, the genes encoding the enzymes disclosed herein are codon optimized for improved expression in the microbial host cell.

The host cells and methods are further suitable for commercial production of cis-3-hexenol, that is, the cells and methods can be productive at commercial scale. In some embodiments, the size of the culture is at least about 100 L, at least about 200 L, at least about 500 L, at least about 1,000 L, or at least about 10,000 L, or at least about 100,000 L. In an embodiment, the culturing may be conducted in batch culture, continuous culture, or semi-continuous culture.

In various embodiments, the microbial host cell is cultured at a temperature between 22° C. and 37° C. While commercial biosynthesis in bacteria such as E. coli can be limited by the temperature at which overexpressed and/or foreign enzymes (e.g., enzymes derived from plants) are stable, recombinant enzymes may be engineered to allow for cultures to be maintained at higher temperatures, resulting in higher yields and higher overall productivity. In some embodiments, the host cell is a bacterial host cell, and culturing is conducted at about 22° C. or greater, about 23° C. or greater, about 24° C. or greater, about 25° C. or greater, about 26° C. or greater, about 27° C. or greater, about 28° C. or greater, about 29° C. or greater, about 30° C. or greater, about 31° C. or greater, about 32° C. or greater, about 33° C. or greater, about 34° C. or greater, about 35° C. or greater, about 36° C. or greater, or about 37° C. or greater, or about 39° C. or greater, or about 40° C. or greater, or about 42° C.

In various embodiments, the microbial host cell is cultured with aeration. The second step in the hexanoic acid to cis-3-hexenol bioconversion pathway is the oxidation of hexanoyl-CoA to trans-2-hexenoyl-CoA by an acyl-CoA oxidase (AOX) enzyme, in a reaction that requires molecular oxygen (O₂) as a source of electrons. Accordingly, the microbial host cell is cultured with oxygen supply. In some embodiments, oxygen-enriched air is supplied to the culture. In some embodiments, oxygen is supplied to the culture.

Cis-3-hexenol can be extracted from media and/or whole cells, and the cis-3-hexenol recovered. In some embodiments, the cis-3-hexenol product is recovered and optionally enriched by fractionation (e.g. fractional distillation). The product can be recovered by any suitable process, including partitioning the desired product into an organic phase. The production of the desired product can be determined and/or quantified, for example, by gas chromatography (e.g., GC-MS). The desired product can be produced in batch or continuous bioreactor systems. Production of product, recovery, and/or analysis of the product can be done as described in US 2012/0246767, U.S. Pat. Nos. 10,501,760, 10,934,564, which are hereby incorporated by reference in its entirety. For example, in some embodiments, cis-3-hexenol is extracted from aqueous culture medium, which may be done by partitioning into an organic phase, followed by fractional distillation. Cis-3-hexenol may be measured quantitatively by GC/MS, followed by blending of the fractions.

In some embodiments, the microbial host cells and methods disclosed herein are suitable for commercial production of cis-3-hexenol, that is, the microbial host cells and methods are productive at commercial scale. In some embodiments, the size of the culture is at least about 100 L, at least about 200 L, at least about 500 L, at least about 1,000 L, at least about 10,000 L, at least about 100,000 L, or at least about 1,000,000 L. In some embodiment, the culturing may be conducted in batch culture, continuous culture, or semi-continuous culture.

In some aspects, the present disclosure provides methods for making a product comprising cis-3-hexenol, including flavor and fragrance compositions or products. In some embodiments, the method comprises producing cis-3-hexenol as described herein through microbial culture, recovering the cis-3-hexenol, and incorporating the cis-3-hexenol into the flavor or fragrance composition, or a consumable product (e.g., a food product).

In one aspect, the present disclosure relates to a method for making cis-3-hexenol. The method comprises culturing the microbial cell of any one of the embodiments disclosed herein, and recovering the cis-3-hexenol from the culture. In some embodiments, the method comprises culturing a microbial cell of any of the embodiments disclosed herein with hexanoic acid. In other embodiments, the hexanoic acid is produced by the cell, which can comprise expression of a biosynthetic pathway comprising one or more heterologous enzymes.

In one aspect, the present disclosure relates to a method for making a product comprising cis-3-hexenol. The method comprises incorporating the cis-3-hexenol, i.e., made according to the method of any of the embodiments disclosed herein or made using a strain described herein, into said product. Examples of products in which highly purified cis-3-hexenol may be used include, but are not limited to, perfumes, cosmetics, food products, beverages, flavors, food additives, fragrances, detergent fragrances, green solvents, antimicrobial ingredients, polymers, nylon precursors, and fuel precursors. In some embodiments, the product is a flavour or fragrance product.

Aspects and embodiments of the invention will now be described according to the following examples,

### EXAMPLES

Cis-3-hexenol (FIG. 1), an unsaturated alcohol, also known as leaf alcohol, is emitted by green plants upon mechanical damage. It is used as a flavoring/aromatic agent. The biosynthesis of cis-3 hexenol according to the present disclosure uses an engineered biosynthetic pathway that synthesizes cis-3 hexenol from hexanoic acid, a product of the fatty acid biosynthesis pathway (FIG. 1).

A pathway for the biosynthesis of cis-3 hexenol was constructed. In this pathway, cis-3 hexenol is synthesized from hexanoic acid (FIG. 1). Briefly, hexanoic acid can be synthesized from acetyl-CoA, which is generated via glycolysis and TCA cycle, via malonoyl-CoA and the action of a type I/type II fatty acid synthase complex (FIG. 1). Hexanoic acid is converted by acyl-CoA synthetase (ACS) to hexanoyl-CoA by an acyl-CoA synthetase (FIG. 1). Hexanoyl-CoA is oxidized by a short chain acyl-CoA oxidase to trans-2-hexenoyl-CoA, which is isomerized to cis-3-hexenoyl-CoA by a enoyl-CoA isomerase (ECI) (FIG. 1). Cis-3-hexenoyl-CoA is reduced by a fatty acyl-CoA reductase (FAR) to cis-3-hexenal, which is reduced by an alcohol dehydrogenase (ADH) to cis-3-hexenol (FIG. 1).

### Example 1. Conversion of Hexanoic Acid to Hexanoyl-CoA

Yarrowia lipolytica strains expressing the following recombinant acyl-CoA synthetase (ACS) enzymes were constructed: SEQ ID NO: 1, SEQ ID NO: 2, SEQ ID NO: 3, SEQ ID NO: 4, SEQ ID NO: 5, SEQ ID NO: 6, or SEQ ID NO: 7. Of these, SEQ ID NO: 6 contained three point mutations (Thr324Gly, Val399Ala, Trp427Gly) and a deletion of an N-terminal peptide compared to the wild type sequence, which has an NCBI accession number NP_001331094.1.

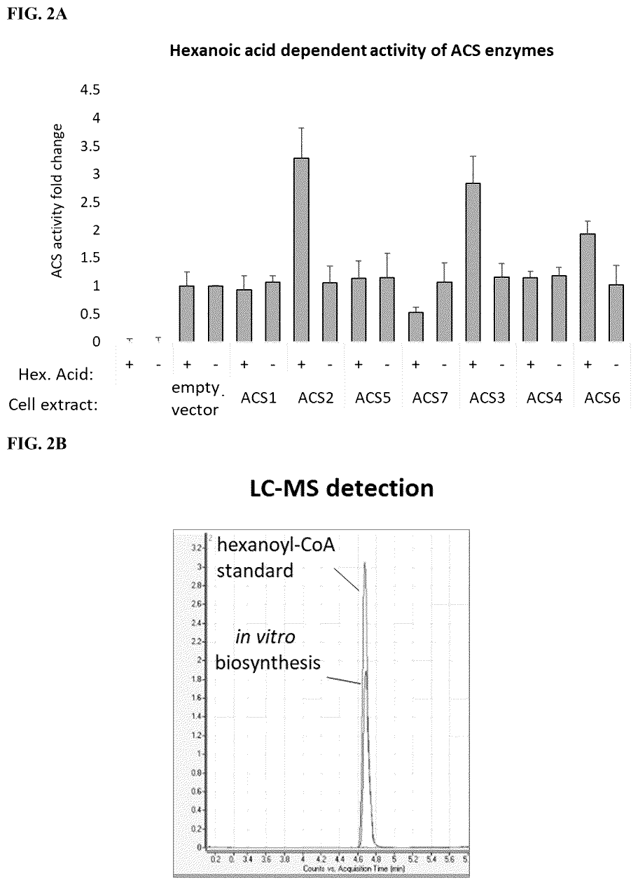

An in vitro assay was carried out using extracts of the Yarrowia lipolytica strains expressing recombinant ACS enzymes and hexanoic acid, ATP and CoA as substrates. Activity was quantified by monitoring consumption of CoA in the reaction mix. A 5,5′-dithiobis(2-nitrobenzoic acid) (DTNB)-coupled colorimetric in vitro assay was used for measuring consumption of CoA and thereby monitoring the ACS activity. Briefly, cell extract was incubated with hexanoic acid, ATP and CoA at room temperature for 30 minutes, and a DTNB-coupled colorimetric measurement of remaining free CoA was performed. The activities of various acyl-CoA synthetase (ACS) enzymes were determined. As shown in FIG. 2A, enzymes of SEQ ID NO: 2, SEQ ID NO: 3, and SEQ ID NO: 6 showed the desired activity. Hexanoyl-CoA synthesis was confirmed using LC-MS in comparison with a hexanoyl-CoA standard (FIG. 2B).

These results demonstrate that an acyl-CoA synthetase (ACS) enzyme is capable of converting hexanoic acid to hexanoyl-CoA.

### Example 2. Conversion of Hexanoyl-CoA to Trans-2-Hexenoyl-CoA

Yarrowia lipolytica strains expressing the following recombinant acyl-CoA oxidase (AOX) enzymes were constructed: SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15), SEQ ID NO: 16, SEQ ID NO: 17, SEQ ID NO: 18, SEQ ID NO: 19; and SEQ ID NO: 20.

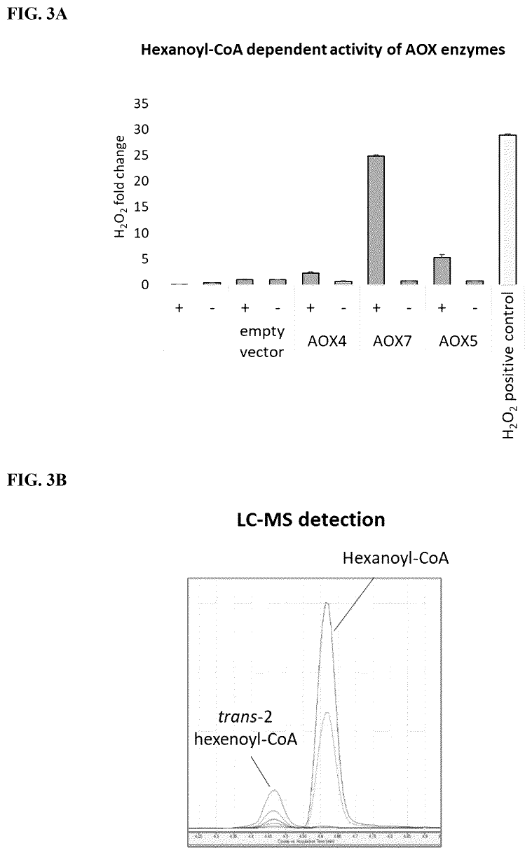

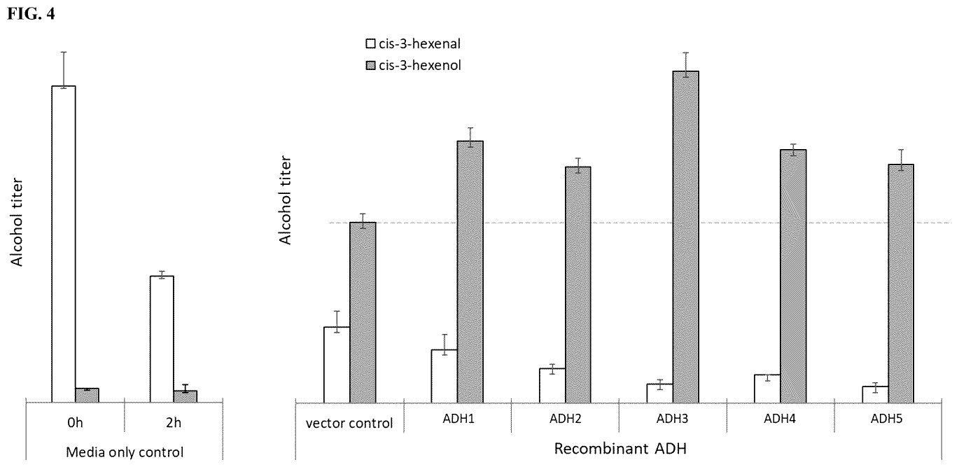

Acyl-CoA oxidase enzymes were screened for the conversion of hexanoyl-CoA to trans-2-hexenoyl-CoA. Towards that, an in vitro assay was performed using extract from Yarrowia lipolytica strains expressing recombinant Acyl-CoA oxidase (AOX) enzymes, and hexanoyl-CoA as a substrate. After a 20 min incubation at room temperature, the activity of recombinant AOX was quantified by the production of H₂O₂ in the reaction mix. A horseradish peroxidase (HRP)-coupled colorimetric in vitro assay was performed for measuring AOX activity. H₂O₂ was used as a positive control for peroxidase activity. As shown in FIG. 3A, activity of AOX enzymes could be detected in the extracts of Yarrowia lipolytica strains expressing SEQ ID NO: 11, SEQ ID NO: 12, and SEQ ID NO: 14. The production of trans-2 hexanoyl-CoA was confirmed by liquid chromatography-mass spectrometry (LC-MS) in comparison with hexanoyl-CoA and trans-2 hexanoyl-CoA standards (FIG. 3B).

### Example 3. Conversion of cis-3-hexenal to cis-3-hexenol

Yarrowia phangngensis strains expressing the following recombinant alcohol dehydrogenase (ADH) enzymes were constructed: SEQ ID NO: 47, SEQ ID NO: 48, SEQ ID NO: 49, SEQ ID NO: 50, and SEQ ID NO: 51.

Alcohol dehydrogenase (ADH) enzymes expressed in Yarrowia phangngensis were screened for the conversion of of cis-3-hexenal to cis-3-hexenol. Towards that, Yarrowia phangngensis strains expressing one of five recombinant ADH enzymes from a replicative plasmid were grown in YDM medium at 30° C. Cells were concentrated in YDM medium supplemented with 500 mg/L cis-3-hexenal and grown in 96-well plate for 2 h at 30° C. Cell cultures were extracted with ethyl acetate and analyzed by GC-FID. As shown in FIG. 4, each of the tested Yarrowia phangngensis strains expressing alcohol dehydrogenase (ADH) enzymes were able to catalyze the conversion of of cis-3-hexenal to cis-3-hexenol. A media-only control is shown on the left-hand side (FIG. 4).

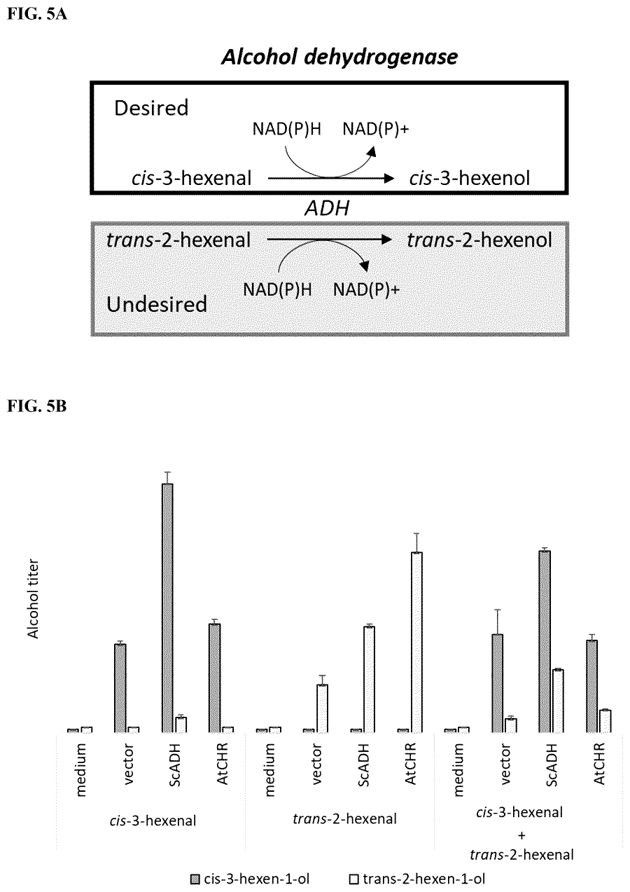

The degree of selectivity for cis-3-hexenal versus trans-2-hexenal was evaluated for enzymes of SEQ ID NO: 47 and SEQ ID NO: 48. FIG. 5A shows the biochemical reactions producing cis-3-hexenol and trans-2-hexenol. Yarrowia lipolytica strains expressing recombinant enzyme were grown in YDM (Yeast Defined Medium) at 30° C. Cells were concentrated in YDM supplemented with 1 g/L of cis-3-hexenal, 1 g/L of trans-2-hexenal or 1 g/L of a mixture of cis-3-hexenal and trans-2-hexenal and grown in 96-well plate for 3 h at 30° C. in presence of an IPM (isopropyl myristate) overlayer. Cell cultures were extracted with ethyl acetate and analyzed by GC-FID. Yarrowia lipolytica strains expressing either enzyme were able to convert both cis-3-hexenal and trans-2-hexenal to cis-3-hexenol and trans-2-hexenol, respectively (See left and middle panels of FIG. 5B. As shown in FIG. 5B (right panel). Yarrowia lipolytica strains expressing the enzyme of SEQ ID NO: 47 showed a selectivity for cis-3-hexenal.

### Example 4. Conversion of Trans-2-hexenoyl-CoA to cis-3-hexenoyl-CoA

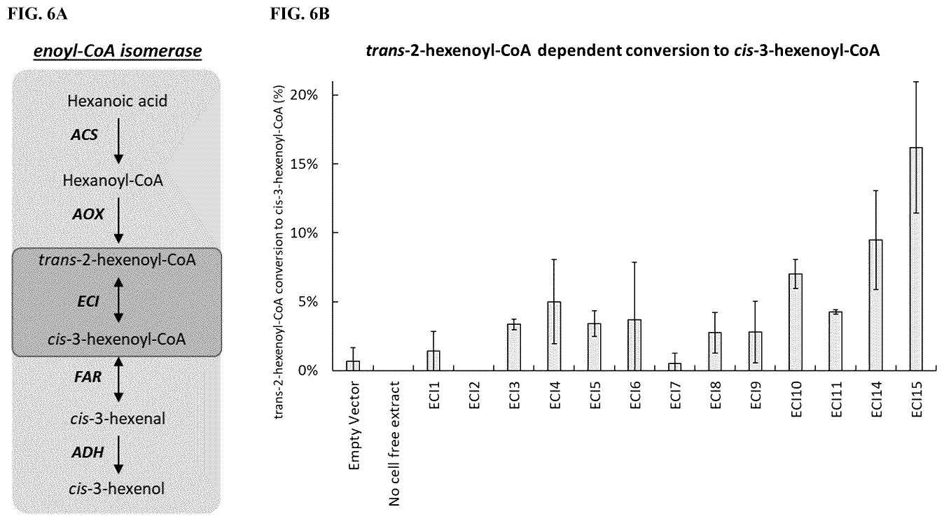

The conversion of trans-2-hexenoyl-CoA to cis-3-hexenoyl-CoA in a synthetic cis-3-hexenol biosynthesis pathway is shown in FIG. 6A. To demonstrate this conversion, Yarrowia lipolytica strains expressing the following enoyl-CoA isomerase (ECI) enzymes were constructed: SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 34; and SEQ ID NO: 35. A Yarrowia lipolytica strain harboring an empty vector was also constructed for use as a control.

The Yarrowia lipolytica strains expressing one of the thirteen enoyl-CoA isomerase (ECI) enzymes from a replicative plasmid and the control strain harboring empty vector were grown in YDM medium at 30° C. Cell free extracts were prepared and incubated with trans-2-hexenoyl-CoA for 30 min at 30° C. The samples were then heat treated to inactivate enzymes and analyzed by LC-MS. As shown in FIG. 6B, cell free extracts of many of the tested Yarrowia lipolytica strains expressing enoyl-CoA isomerase (ECI) enzymes were able to catalyze increased conversion of trans-2-hexenoyl-CoA to cis-3-hexenoyl-CoA compared to the cell free extract of the control strain harboring an empty vector. Among the tested ECI enzymes, SEQ ID NO: 30, SEQ ID NO: 34; and SEQ ID NO: 35 were the three best enzymes that were able to convert trans-2-hexenoyl-CoA to cis-3-hexenoyl-CoA when expressed in Yarrowia lipolytica (FIG. 6B). These results demonstrate that several enoyl-CoA isomerase (ECI) enzymes were capable of converting trans-2-hexenoyl-CoA to cis-3-hexenoyl-CoA when expressed in Yarrowia lipolytica.

### Example 5. Conversion of cis-3-hexenoyl-CoA to cis-3-hexenal

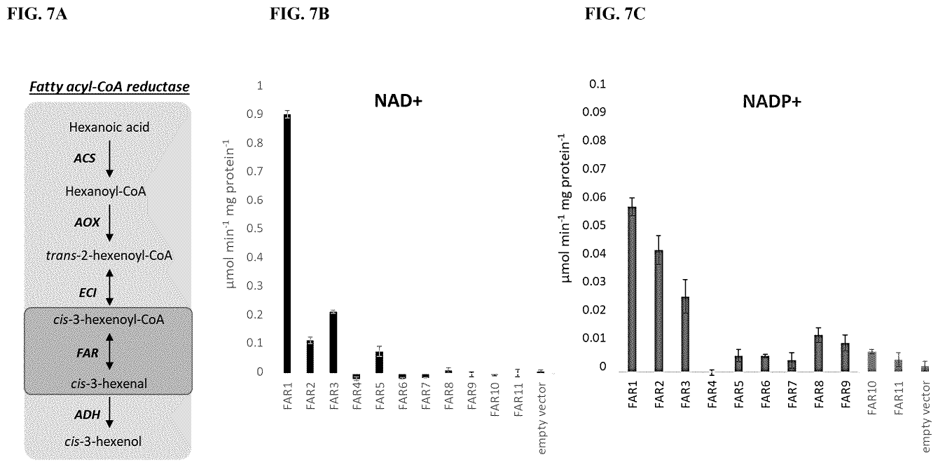

The conversion of cis-3-hexenoyl-CoA to cis-3-hexenal in the synthetic cis-3-hexenol biosynthesis pathway is shown in FIG. 7A. To demonstrate this conversion, Yarrowia lipolytica strains expressing the following fatty acyl-CoA reductase (FAR) enzymes were constructed: SEQ ID NO: 36, SEQ ID NO: 37, SEQ ID NO: 38, SEQ ID NO: 39, SEQ ID NO: 40, SEQ ID NO: 41, SEQ ID NO: 42; SEQ ID NO: 43, SEQ ID NO: 44, SEQ ID NO: 45, and SEQ ID NO: 46. A Yarrowia lipolytica strain harboring an empty vector was also constructed for use as a control.

Conversion of cis-3-hexenal to cis-3-hexenoyl-CoA is a reversible reaction. Accordingly, the enzymes that can convert cis-3-hexenal to cis-3-hexenoyl-CoA were identified using an assay that measures reduction of NAD+ or NADP+. Briefly, the Yarrowia lipolytica strains expressing one of the eleven fatty acyl-CoA reductase (FAR) enzymes from a replicative plasmid and the control strain harboring an empty vector were grown in YDM medium at 30° C. Cell free extracts were prepared. Reactions comprising a cell free extract, cis-3-hexenal, CoA, and an electron-pair recipient cofactor were assembled. The electron-pair recipient cofactors used in this experiment were NAD+ and NADP+. Production of NADH or NADPH was assayed by measuring absorbance at 340 nm.

FIG. 7B shows the conversion of cis-3-hexenal to cis-3-hexenoyl-CoA by the cell extracts in the presence of NAD+, as measured by absorbance at 340 nm. As shown in FIG. 7B, cell free extracts of many of the tested Yarrowia lipolytica strains expressing fatty acyl-CoA reductase (FAR) enzymes were able to catalyze the conversion of cis-3-hexenal to cis-3-hexenoyl-CoA compared to the cell free extract of the control strain harboring empty vector. Among the tested FAR enzymes, SEQ ID NO: 36, SEQ ID NO: 37, and SEQ ID NO: 38 were the three best enzymes that were able to convert cis-3-hexenal to cis-3-hexenoyl-CoA using NAD+ as a cofactor when expressed in Yarrowia lipolytica (FIG. 7B). These results demonstrate that several fatty acyl-CoA reductase (FAR) enzymes were capable of converting cis-3-hexenal to cis-3-hexenoyl-CoA when expressed in Yarrowia lipolytica. Since this reaction is reversible, these data also show that several fatty acyl-CoA reductase (FAR) enzymes (e.g., SEQ ID NO: 36, SEQ ID NO: 37, and SEQ ID NO: 38) can convert cis-3-hexenoyl-CoA to cis-3-hexenal when substrates cis-3-hexenoyl-CoA and NADH are in excess.

FIG. 7C shows the conversion of cis-3-hexenal to cis-3-hexenoyl-CoA by the cell extracts in the presence of NADP+, as measured by absorbance at 340 nm. As shown in FIG. 7C, cell free extracts of many of the tested Yarrowia lipolytica strains expressing fatty acyl-CoA reductase (FAR) enzymes were able to catalyze conversion of cis-3-hexenal to cis-3-hexenoyl-CoA compared to the cell free extract of the control strain harboring empty vector. Among the tested FAR enzymes, SEQ ID NO: 36, SEQ ID NO: 37, and SEQ ID NO: 38 were the three best enzymes that were able to convert cis-3-hexenal to cis-3-hexenoyl-CoA using NADP+ as a cofactor when expressed in Yarrowia lipolytica (FIG. 7C). These results demonstrate that several fatty acyl-CoA reductase (FAR) enzymes were capable of converting cis-3-hexenal to cis-3-hexenoyl-CoA when expressed in Yarrowia lipolytica. Since this reaction is reversible, these data also show that several fatty acyl-CoA reductase (FAR) enzymes (e.g., SEQ ID NO: 36, SEQ ID NO: 37, and SEQ ID NO: 38) are capable of converting cis-3-hexenoyl-CoA to cis-3-hexenal when substrates cis-3-hexenoyl-CoA and NADPH are in excess.

### Example 6. Background Strain Engineering for the Production of cis-3-Hexenol

For cis-3-hexenol biosynthesis, e.g., a yeast cell may be grown using sugar as a carbon source and/or hexanoic acid. In some embodiments, sugar is converted to biomass through glycolysis or funneled through the pentose phosphate pathway to hexanoic acid. In some embodiments, hexanoic acid is provided externally. The yeast cell expresses the cis-3-hexenol biosynthetic pathway, and comprises one or more biosynthetic pathways to improve flux through the biosynthetic pathway, and/or reduce degradation of product. In some embodiments, H₂O₂ detoxification enzymes are overexpressed (e.g., cytosolic catalase, CTT1; and/or peroxisomal catalase, CTA1). Alternatively or in addition, genetic modifications can increase glycolytic flux through the pentose phosphate pathway to increase NADPH supply. Exemplary modifications include deletion, inactivation, or reduced expression of glucose-6-phosphate isomerase (e.g., PGI1) and/or phosphofructokinase (e.g., PFK1); or overexpression or increased activity of glucose-6-phosphate dehydrogenase (e.g., ZWF1) and/or 6-phosphogluconate dehydrogenase (e.g., GND1). Alternatively or in addition, genetic modifications expressing a heterologous NADPH biosynthetic route, such as expression of bacterial transhydrogenase (e.g., pntAB) and/or bacterial or plant NADP-dependent glyceraldehyde-3-phosphate dehydrogenase (e.g., gapN). Alternatively or in addition, genetic modifications can enhance NADPH supply by overexpression of eukaryotic cytosolic NADP (+)-dependent isocitrate dehydrogenase (IDH). Alternatively or in addition, genetic modifications can downregulate, delete, or inactivate β-oxidation or peroxisome enzymes, such as a multifunctional β-oxidation enzyme (e.g., MFE1), a peroxisomal membrane E3 ubiquitin ligase (e.g., PEX10), a peroxisomal membrane protein (e.g., PEX11), one or more peroxisomal acyl-CoA oxidases (e.g., POX1, POX2, POX3, POX4, POX5, and POX6), and/or a peroxisomal adenine nucleotide transporter (e.g., ANT1). Alternatively or in addition, genetic modifications can reduce formation of by-products, for example, by deletion, inactivation, or reduced expression of citric acid cytoplasmic exporter (e.g., CEX1) and/or one or more NADPH-dependent aldehyde reductases (e.g., ALR1-12). Alternatively or in addition, genetic modifications can include deletion, inactivation, or reduced expression or activity of one or more neutral lipid biosynthesis genes (e.g., diacylglycerol acyltransferases DGA1, DGA2, and LRO1; and or acyl-CoA:sterol acyltransferase ARE1). Alternatively or in addition, genetic modifications include modified expression or activity of alcohol dehydrogenases and/or alcohol oxidase (e.g., ADH1, ADH2, ADH3, ADH4, ADH5, ADH6, ADH7, FADH, and FAO1).

### Example 7. Response of Yarrowia lipolytica Transcriptome to the Heterologous cis-3-Hexenol Production Pathway

In order to identify genes that are differentially expressed in the presence of the heterologous cis-3-hexenol production pathway, a RNAseq experiment was performed on a MFE1ΔY. lipolytica strain and a MFE1Δ ACS2-AOX5-EC115-FAR1-ADH1 cis-3-hexenol pathway strain in media with either glucose or glucose and hexanoic acid as carbon sources. After 16 hours of incubation, cells were harvested and RNA was extracted for sequencing. Transcript values were processed using standard bioinformatics methods and reported in values of transcript reads per million (TPM). Among notable changes were upregulation of some aldehyde dehydrogenases that convert aldehydes into carboxylic acids. These aldehyde dehydrogenases included YALI0C03025g or YALI0F04444g. Additionally, some alcohol dehydrogenases, which may either convert aldehydes to alcohols or convert alcohols to aldehydes, were differentially regulated.

### Example 8. Bioconversion of hexanoic acid to cis-3-hexenol in Yarrowia lipolytica

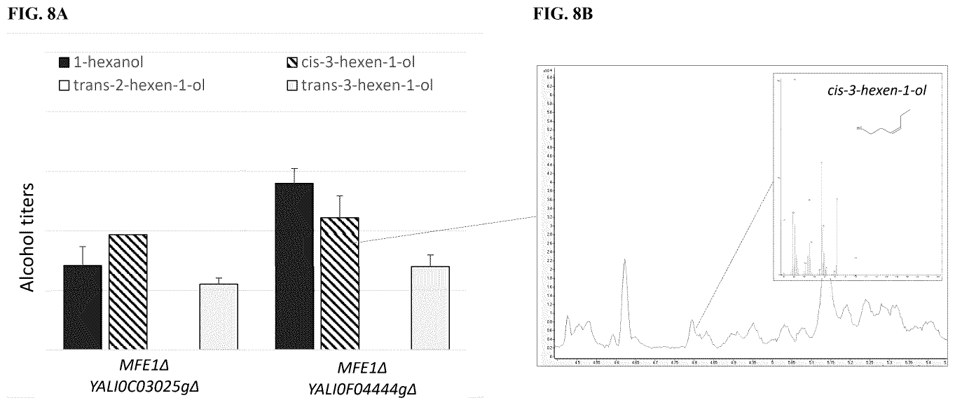

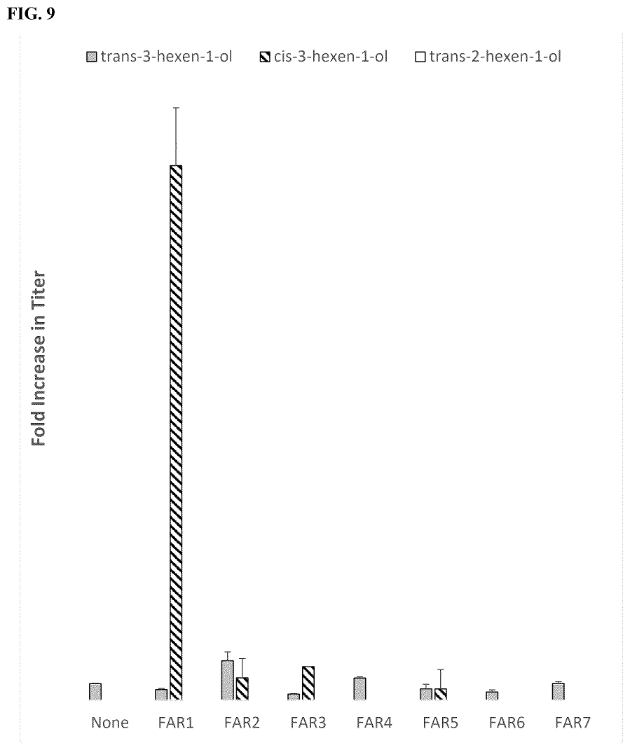

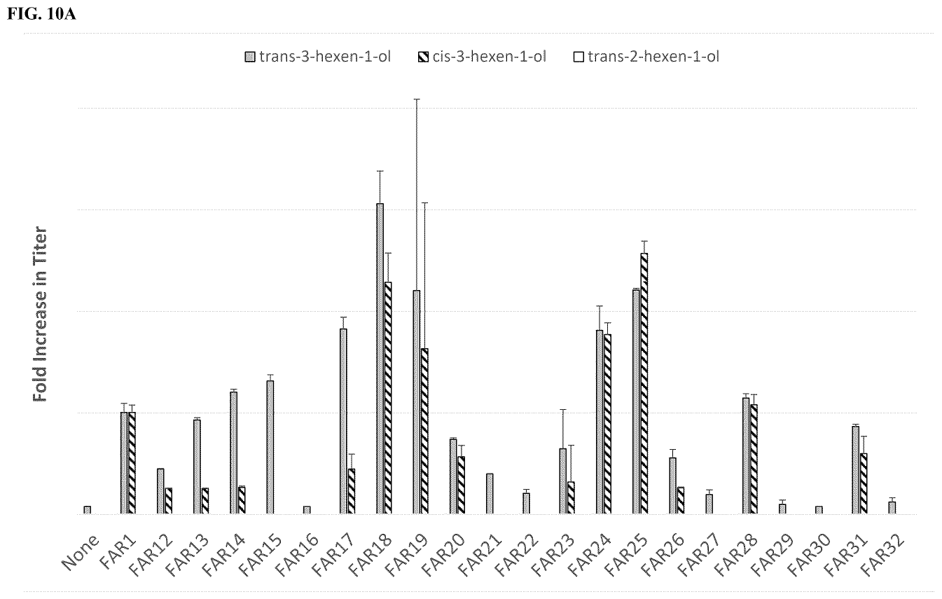

To evaluate the feasibility of in vivo hexanoic acid to cis-3-hexenol production in Yarrowia lipolytica, a heterologous metabolic pathway was constructed using the ACS2 (SEQ ID NO: 2), AOX5 (SEQ ID NO: 12), ECI15 (SEQ ID NO: 35), FAR1 (SEQ ID NO: 36), and ADH1 (SEQ ID NO: 47) genes expressed by constitutive promoters. This pathway was integrated into the genome of two Y. lipolytica strains that additionally had deletions of the MFE1 and the aldehyde dehydrogenases YALI0C03025g or YALI0F04444g. These strains were cultivated in medium containing 20 g/L glucose and 3 g/L hexanoic acid for 48 h in a 96-well plate. Medium broth was extracted with ethyl acetate and analyzed by GC-FID and GC-MS to detect the presence of organic alcohols. FIG. 8A shows the titers of various alcohols in strains having the deletion of YALI0C03025g or YALI0F04444g. FIG. 8B shows the GC-FID profile showing the production of various alcohols. The production of cis-3-hexenol was demonstrated using GC-MS profile (see FIG. 8B inset). Both strains were observed to produce 1.9-2.2 mg/L cis-3-hexenol, in addition to 1-hexanol and trans-3-hexenol, which was higher compared to the parental YALI0C03025g or YALI0F04444g strain. There results show the improved production of cis-3-hexenol from hexanoic acid by Yarrowia lipolytica strain having deletions of aldehyde dehydrogenase genes that are overexpressed in the strains that carry the recombinant cis-3-hexenol pathway.

### Example 9. Bioconversion of Hexanoic Acid to cis-3-Hexenol in Escherichia coli

To evaluate the feasibility of in vivo hexanoic acid to cis-3-hexenol production in Escherichia coli, a heterologous metabolic pathway was constructed using the ACS2 (SEQ ID NO: 2), AOX5 (SEQ ID NO: 12), ECI15 (SEQ ID NO: 35), FAR1 (SEQ ID NO: 36) and ADH1 (SEQ ID NO: 47) genes expressed by constitutive and inducible promoters. These genes were expressed in an Escherichia coli strain with deletions in genes fadB, fadJ and paaZ. The strain was cultivated in medium containing 20 g/L glycerol and 3.6 g/L hexanoic acid for 24 h in a 96-well plate. Fermentation culture was extracted using ethyl acetate and analyzed by GC-FID for the detection of organic alcohols. Production of cis-3-hexenol was achieved in this basal strain expressing FAR1, in addition to trans-3-hexenol. These results showed that the engineered pathway functionally expresses and produces cis-3-hexenol in diverse hosts.

### Example 10. Background Strain Engineering for the Production of cis-3-Hexenol

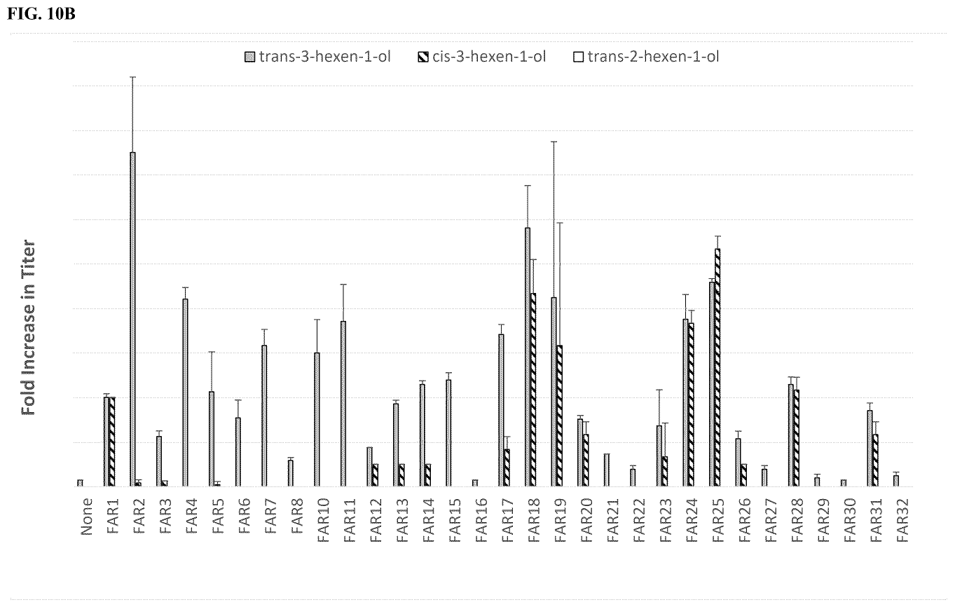

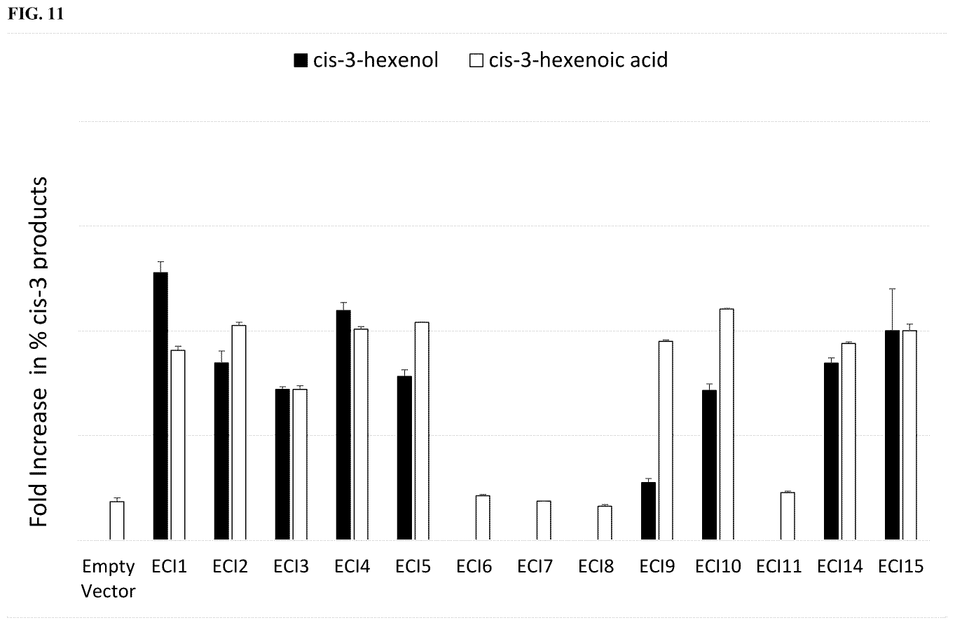

To improve cis-3-hexenol production in Escherichia coli, a panel of FAR enzymes (FAR12-32, SEQ ID NO: 52-72) was expressed from an inducible promoter along with ECI15 (SEQ ID NO: 35) and ADH1 (SEQ ID NO: 47), in a strain where ACS2 (SEQ ID NO: 2) and AOX5 (SEQ ID NO: 12) were expressed from a constitutive promoter. The Escherichia coli strain was deleted for genes fadB, fadJ and paaZ. The strains were grown in medium containing 20 g/L glycerol and 3.6 g/L hexanoic acid for 24 h in a 96-well plate. Culture was extracted using ethyl acetate and analyzed by GC-FID for the detection of organic alcohols and carboxylic acids. The strain expressing FAR1 (SEQ ID NO: 36) is shown on the left-hand side of the figure as a positive control. Several strains produced higher cis-3-hexenol titers than the strain expressing FAR1; namely, the strains with FAR18 (SEQ ID NO: 58), FAR19 (SEQ ID NO: 59), FAR24 (SEQ ID NO: 64), FAR25 (SEQ ID NO: 65), and FAR28 (SEQ ID NO: 69) (FIG. 9A and FIG. 10). achieved an increase in cis-3-hexenol production by up to 2.67-fold. These strains also produced trans-3-hexenol to varying levels.

### Example 11. Evaluation of Enoyl-CoA Isomerase (ECI) Activity and Selectivity for cis-3-Hexenol Production from the Bioconversion of Hexanoic Acid in Escherichia coli

A panel of ECI enzymes (ECI1-14 (SEQ ID NOs: 21-34) were screened in Escherichia coli to evaluate the selectivity exhibited in their isomerization of the pathway intermediate trans-2-hexenoyl-CoA to cis-3-hexenoyl-CoA, or the unwanted product trans-3-hexenoyl-CoA, relative to ECI15 (SEQ ID NO: 35). This ECI panel was expressed from an inducible promoter with FAR1 (SEQ ID NO: 36) and ADH1 (SEQ ID NO: 47) in an Escherichia coli strain expressing ACS2 (SEQ ID NO: 2) and AOX5 (SEQ ID NO: 12) constitutively. The strains were grown in medium containing 20 g/L glycerol and 3.6 g/L hexanoic acid for 24 h in a 96-well plate. Culture was extracted using ethyl acetate and analyzed by GC-FID for the detection of organic alcohols and carboxylic acids. The relative selectivity of the ECI enzymes was evaluated through the calculation of the % cis-3-hexenol among the total unsaturated six-carbon alcohol (cis-3-hexenol, trans-3-hexenol, and trans-2-hexenol) compounds detected, as well as the % cis-3-hexenoic acid among the total unsaturated six-carbon carboxylic acid (cis-3-hexenoic acid, trans-3-hexenoic acid, and trans-2-hexenoic acid) compounds detected. As shown in FIG. 11, the strain expressing ECI1 (SEQ ID NO: 21) gave a 1.29× increase % cis-3-hexenol selectivity.

### Example 12. Enhanced Unsaturated Six-Carbon Carboxylic Acid Production from Hexanoic Acid Through Modulation of Pathway Enzyme Expression Levels

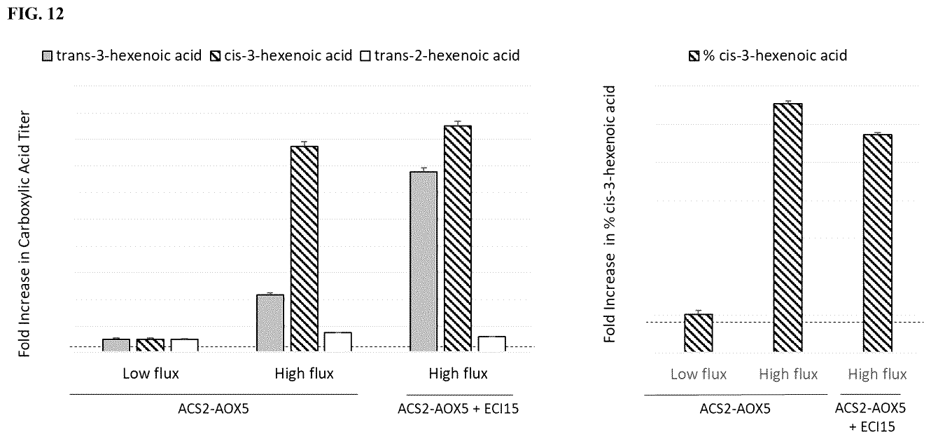

The first three steps of hexanoic acid to cis-3-hexenol bioconversion pathway constitute: first, the production of hexanoyl-CoA from hexanoic acid by an ACS enzyme; second, oxidation to trans-2-hexenoyl-CoA by an AOX enzyme; and third, isomerization of trans-2-hexenoyl-CoA to cis-3-hexenoyl-CoA by an ECI enzyme. In this example, the impact of gene expression level on these steps of the pathway was assessed through the production of trans-2-hexenoic acid, cis-3-hexenoic acid and trans-3-hexenoic acids as hydrolysis products from their respective coenzyme A-conjugated species. Here, Escherichia coli with genetic deletions in fadB, fadJ and paaZ expressed ACS2 (SEQ ID NO: 2) and AOX5 (SEQ ID NO: 12) in either low expression level (low flux) or high expression level (high flux) contexts, in the presence or absence of ECI15 (SEQ ID NO: 35). The strains were grown in medium containing 20 g/L glycerol and 1 g/L hexanoic acid for 24 h in a 96-well plate. Fermentation culture was extracted using ethyl acetate and analyzed by GC-FID for the detection of carboxylic acids. In the high flux context, where ACS2 (SEQ ID NO: 2) and AOX5 (SEQ ID NO: 12) are highly expressed, cis-3-hexenoic acid titers increased 15.4-fold compared to the low flux context. In this high flux context, the percentage of cis-3-hexenoic acid within the total unsaturated six-carbon carboxylic acid levels increased 6.5-fold. Although cis-3-hexenoic acid titers increase 17-fold by overexpression of ECI15 (SEQ ID NO: 35, (see FIG. 12 (left panel)), it reduces cis-3-hexenoic acid percentage to 5.7-fold due to a concomitant increase in trans-3-hexenoic acid production. (See FIG. 12 (right panel))

### Example 13. Screen for Native ECI Activities from Escherichia coli for Activity in the cis-3-Hexenol Bioconversion Pathway

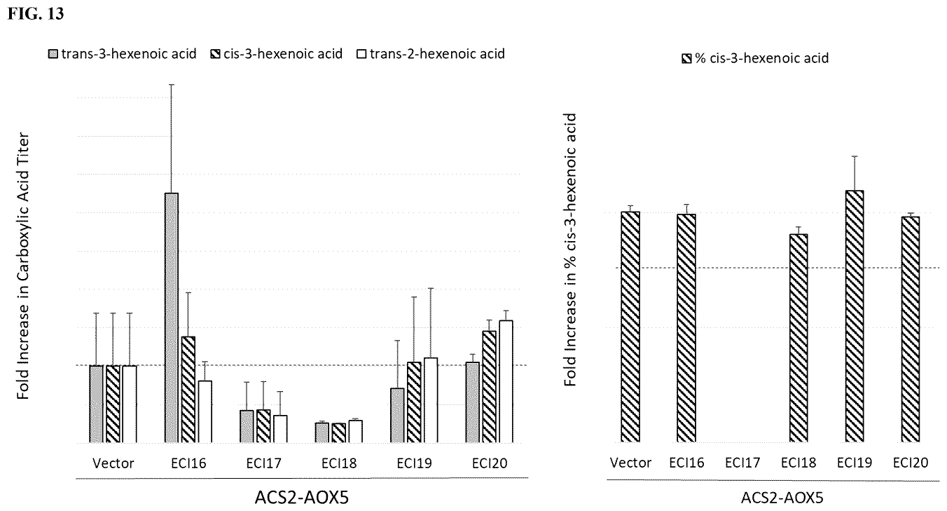

The results from screening our initial ECI panel suggests that high expression levels of heterologous ECI enzymes produced significant trans-3-hexenoyl-CoA from trans-2-hexenoyl-CoA in the cis-3-hexenol bioconversion pathway from hexanoic acid in Escherichia coli. However, the production of cis-3 and trans-3 species were also observed in the absence of a heterologous ECI enzyme, suggesting E. coli contains additional enzymes that can catalyze this conversion in addition to the previously reported activity of the FadB and FadJ enzymes, which were deleted in this strain. Five native genes encoding putative isomerase enzymes (ECI16-20, SEQ ID NOs: 73-77) were screened for activity within the cis-3-hexenol bioconversion pathway. This ECI panel was expressed from a constitutive promoter in an Escherichia coli strain that harbors ACS2 (SEQ ID NO: 2) and AOX5 (SEQ ID NO: 12) under the control of an inducible promoter. The Escherichia coli strain harbored deletions in the genes fadB, fadJ and paaZ. The strains were grown in medium containing 20 g/L glycerol and 1 g/L hexanoic acid for 24 h in a 96-well plate. The expression of ACS2 (SEQ ID NO: 2) and AOX5 (SEQ ID NO: 12) was modulated by the absence or addition of inducer compound to evaluate the activity of the ECI enzymes in the presence of minimal or high levels of the pathway intermediates hexanoyl-CoA and trans-2-hexanoyl-CoA. Culture was extracted using ethyl acetate and analyzed by GC-FID for the detection of carboxylic acids. As shown, the strong constitutive expression of ECI16 (SEQ ID NO: 73) increased the production of trans-3- and cis-3-hexenoic acids (see FIG. 13 (left panel)), without altering the proportion of cis-3-hexenoic acid therein (see FIG. 13 (right panel)).

### Example 14. High Aeration Increases cis-3-hexenol Titers in the Hexanoic Bioconversion Pathway in Escherichia coli

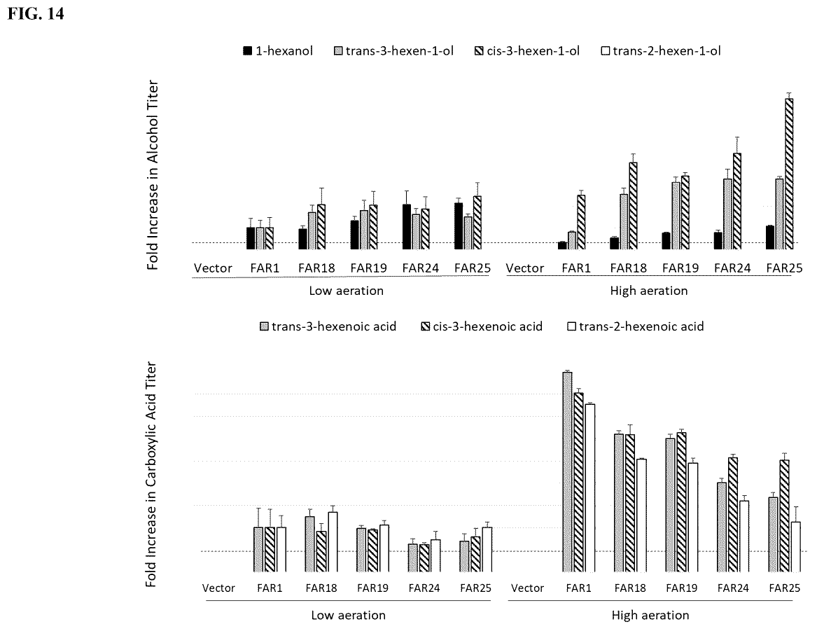

The second step in the hexanoic acid to cis-3-hexenol bioconversion pathway is the oxidation of hexanoyl-CoA to trans-2-hexenoyl-CoA by an acyl-CoA oxidase (AOX) enzyme, in a reaction that requires molecular oxygen. We evaluated the impact of aeration on cis-3-hexenol production in Escherichia coli expressing the bioconversion pathway. Here, ACS2 (SEQ ID NO: 2) and AOX5 (SEQ ID NO: 12) were constitutively expressed, and ECI15 (SEQ ID NO: 35), FAR, and ADH1 (SEQ ID NO: 47) were under the control of an inducible promoter. The FAR genes expressed were FAR1 (SEQ ID NO: 36), FAR18 (SEQ ID NO: 58), FAR19 (SEQ ID NO: 59), FAR24 (SEQ ID NO: 64), and FAR25 (SEQ ID NO: 65). The equivalent strain lacking a FAR gene was included as a negative control. The Escherichia coli strain harbored deletions in the genes fadB, fadJ and paaZ. The strains were grown in medium containing 20 g/L glycerol and 1 g/L hexanoic acid for 24 h in a 96-well plate with either low or high agitation to facilitate low or high aeration of the cultures. Culture was extracted using ethyl acetate and analyzed by GC-FID for the detection of organic alcohols and carboxylic acids. As shown in FIG. 14 (top panel), in comparison to strains cultivated at low aeration, high aeration cultivation produces a 2.5-fold improvement in cis-3-hexenol production by the strain expressing FAR1 (SEQ ID NO: 36), and 4.0-7.0-fold by expressing FAR18, FAR19, FAR24 or FAR25 (SEQ ID NOs: 58, 59, 64 and 65). In addition, these FARs increase the production of cis-3-hexenoic acid by 2.5-4.0-fold at high aeration compared to FAR1 at low aeration (FIG. 14 (bottom panel)).

### Example 15. Cultivation Temperature Changes the Unsaturated Six-Carbon Compound Profile Produced by the cis-3-hexenol Bioconversion Pathway in Escherichia coli

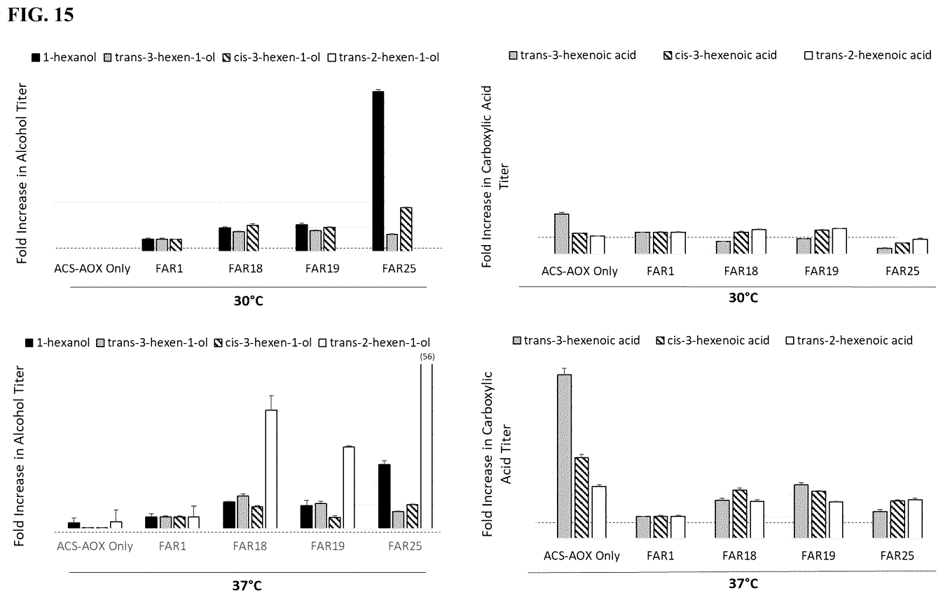

The heterologous metabolic pathway for the bioconversion of hexanoic acid to cis-3-hexenol was introduced into an Escherichia coli strain engineered with deletions in the genes fadB, fadJ and paaZ. This pathway consists of ACS2 (SEQ ID NO: 2) and AOX5 (SEQ ID NO: 12) expressed from an inducible promoter to achieve high flux in the entry steps of the pathway. A panel of FAR enzymes was also expressed from an inducible promoter, while endogenous expression levels of each of ECI16-20 (SEQ ID NOs: 73-77) were maintained in all strains. The FAR panel consisted of FAR1 (SEQ ID NO: 36), FAR18 (SEQ ID NO: 58), FAR19 (SEQ ID NO: 59), FAR24 (SEQ ID NO: 64), and FAR25 (SEQ ID NO: 65). The strains were grown in medium containing 20 g/L glycerol and 1 g/L hexanoic acid for 24 h in a 96-well plate at either 30° C. or 37° C. Culture was extracted using ethyl acetate and analyzed by GC-FID for the detection of organic alcohols (FIG. 15 (left panels)) and carboxylic acids (FIG. 15 (right panels)). Cultivation of the bioconversion strains at 37° C. promoted the production of trans-2-hexenol from strains expressing FAR18, FAR19 and FAR25 (compare FIG. 15 (top panels) with FIG. 15 (bottom panels)).

### Example 16. In Vivo Production of Hexanoic Acid in E. Coli

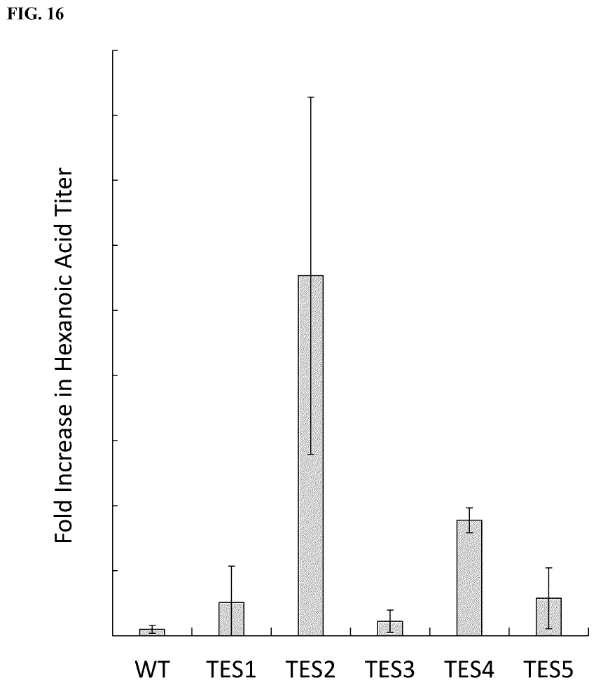

To evaluate the feasibility of in vivo hexanoic acid production in Escherichia coli, a panel of five acyl-acyl carrier protein (ACP) thioesterases (TES) TES1, TES2, TES3, TES4, and TES5 (SEQ ID NOs: 78-82) were expressed from a constitutive promoter on replicating vector in a ΔfadD E. coli strain. Strains were cultivated in LB medium with 0.4% glycerol for 24 hours at 30° C., and then the fermentation broth was extracted with ethyl acetate and analyzed by GC-MS for the presence of hexanoic acid. As shown in FIG. 16, the highest producers, TES2 (SEQ ID NO: 79) and TES4 (SEQ ID NO: 81), produced 24 mg/L and 8 mg/L hexanoic acid in these conditions, demonstrating the ability to directly synthesize hexanoic acid in E. coli from common metabolic precursors.

### Example 17. Identification of Six-Carbon Products and Intermediates from the Hexanoic Acid to cis-3-Hexenol Bioconversion Metabolic Pathway

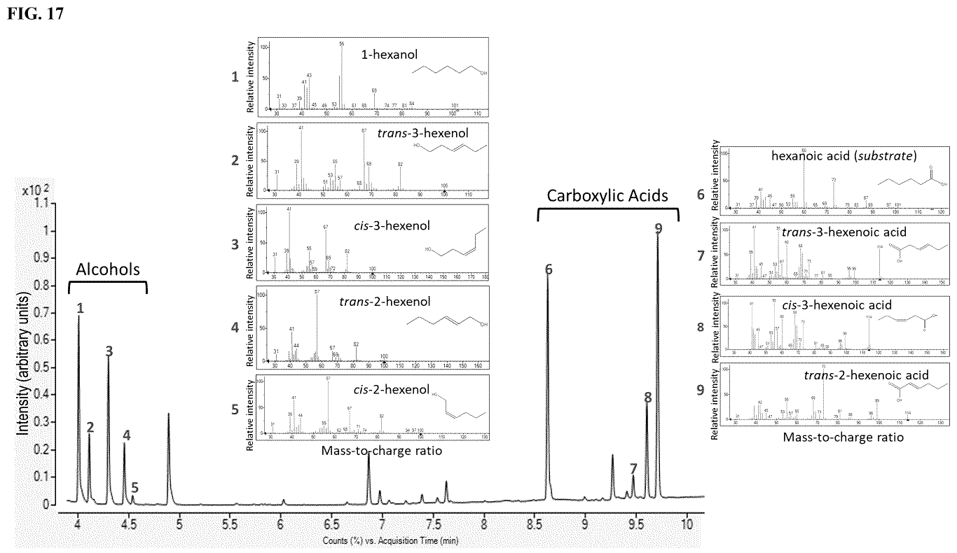

The identification of compounds produced in Escherichia coli from the bioconversion of hexanoic acid to cis-3-hexenol was achieved by the analysis of fermentation extracts by GC-MS. An Escherichia coli strain deleted for the genes fadB, fadJ and paaZ was engineered to express ACS2 (SEQ ID NO: 2), AOX5 (SEQ ID NO: 12), FAR25 (SEQ ID NO: 65) and ADH1 (SEQ ID NO: 47) from inducible promoters, and maintain endogenous expression levels of each of ECI16-20 (SEQ ID NOs: 73-77). The strain was cultivated for 24 h in medium broth supplemented with 20 g/L glycerol and 1 g/L hexanoic acid in a 96-well plate. Culture was extracted using ethyl acetate and analyzed by GC-MS for the detection of organic alcohols and carboxylic acids. As shown in FIG. 17, the organic pathway intermediates could be detected.

### Example 18. Overexpression of Native ADH Enzymes Impacts Six-Carbon Alcohol Production

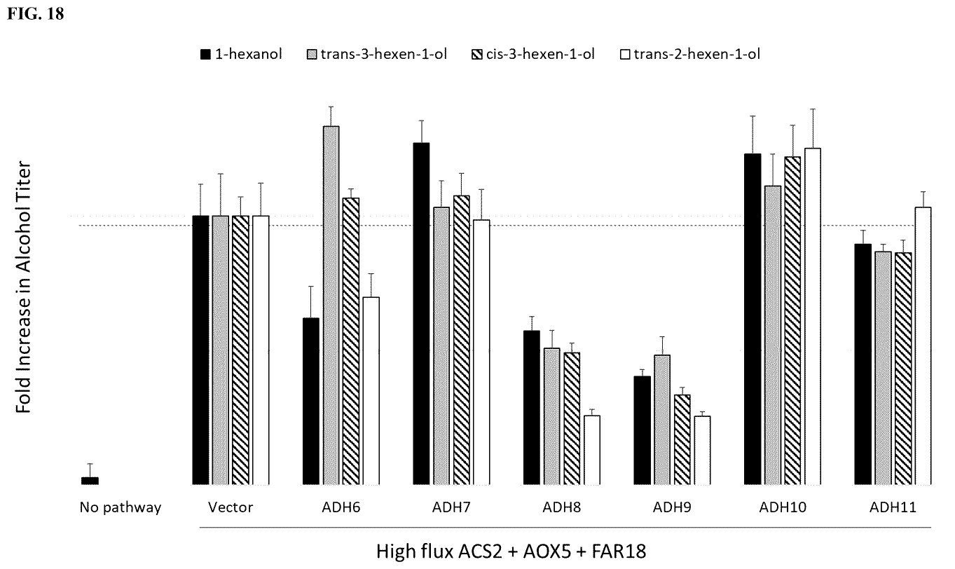

The ability of native Escherichia coli alcohol dehydrogenase (ADH) enzymes to increase or decrease the production of cis-3-hexenol and other six-carbon alcohols was evaluated. A partial metabolic pathway for the bioconversion of hexanoic acid to cis-3-hexenol was introduced into an Escherichia coli strain engineered with deletions in the genes fadB, fadJ and paaZ. This pathway consists of ACS2 (SEQ ID NO: 2) and AOX5 (SEQ ID NO: 12) expressed from an inducible promoter to achieve high flux in the entry steps of the pathway. Endogenous expression levels of each of ECI16-20 (SEQ ID NOs: 73-77) were maintained in all strains, and FAR18 (SEQ ID NO: 58), was expressed from an inducible promoter. A panel of native ADH enzymes (ADH6-11, SEQ ID NOs: 83-88) was constitutively expressed. The strains were grown in medium containing 20 g/L glycerol and 1 g/L hexanoic acid for 24 h in a 96-well plate. Culture was extracted using ethyl acetate and analyzed by GC-FID for the detection of organic alcohols. As shown in FIG. 18, the overexpression of native ADH genes alters unsaturated six-carbon alcohol production. Specifically, overexpression of ADH6 (SEQ ID NO: 83), ADH7 (SEQ ID NO: 84) and ADH10 (SEQ ID NO: 87) improved the production of one or more organic alcohols (FIG. 18).

### Example 19. Replacement of fabI with ENR1 Increases Unsaturated Carboxylic Acid Production

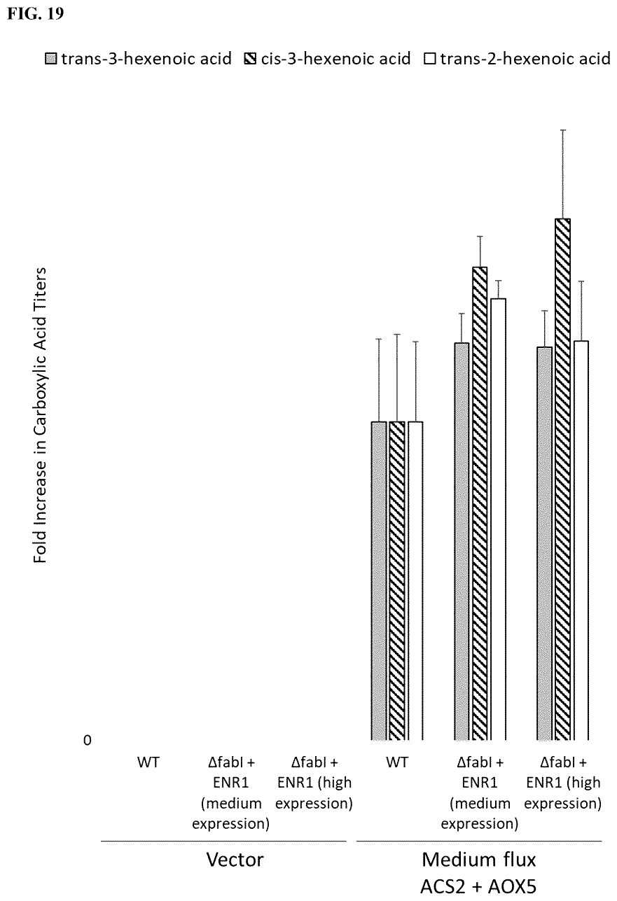

An Escherichia coli strain with deletions in fadB, fadJ and paaZ expressed ACS2 (SEQ ID NO: 2) and AOX5 (SEQ ID NO: 12) from an inducible promoter to achieve high flux in the entry steps of the pathway for the bioconversion of hexanoic aid to cis-3-hexenol. The native fabI gene was deleted from the Escherichia coli chromosome and replaced by the enoyl-ACP reductase ENR1 (SEQ ID NO: 89) under a medium- or high-level constitutive promoter. As shown in FIG. 19, through this replacement a 1.5-1.7-fold increase in cis-3-hexenoic acid levels was achieved.

SEQUENCES
Acyl-CoA synthetases (ACS)
SEQ ID NO: 1
Escherichia coli ecFadK (ACS1)
MHPTGPHLGPDVLFRESNMKVTLTENEQRRAAYRQQGLWGDASLADYWQQTARAMPDKIAVVDNHG
ASYTYSALDHAASCLANWMLAKGIESGDRIAFQLPGWCEFTVIYLACLKIGAVSVPLLPSWREAEL
VWVLNKCQAKMFFAPTLFKQTRPVDLILPLQNQLPQLQQIVGVDKLAPATSSLSLSQIIADNTSLT
TAITTHGDELAAVLFTSGTEGLPKGVMLTHNNILASERAYCARLNLTWQDVEMMPAPLGHATGELH
GVTAPFLIGARSVLLDIFTPDACLALLEQQRCTCMLGATPFVYDLLNVLEKQPADLSALRFFLCGG
TTIPKKVARECQQRGIKLLSVYGSTESSPHAVVNLDDPLSREMHTDGYAAAGVEIKVVDDARKTLP
PGCEGEEASRGPNVEMGYFDEPELTARALDEEGWYYSGDLCRMDEAGYIKITGRKKDIIVRGGENI
SSREVEDILLQHPKIHDACVVAMSDERLGERSCAYVVLKAPHHSLSLEEVVAFFSRKRVAKYKYPE
HIVVIEKLPRTTSGKIQKELLRKDIMRRLTQDVCEEIE
SEQ ID NO: 2
Pseudomonas putida ppLvaE (ACS2)
MMVPTLEHELAPNEANHVPLSPLSFLKRAAQVYPQRDAVIYGARRYSYRQLHERSRALASALERVG
VQPGERVAILAPNIPEMLEAHYGVPGAGAVLVCINIRLEGRSIAFILRHCAAKVLICDREFGAVAN
QALAMLDAPPLLVGIDDDQAERADLAHDLDYEAFLAQGDPARPLSAPQNEWQSIAINYTSGTTGDP
KGVVLHHRGAYLNACAGALIFQLGPRSVYLWTLPMFHCNGWSHTWAVTLSGGTHVCLRKVQPDAIN
AAIAEHAVTHLSAAPVVMSMLIHAEHASAPPVPVSVITGGAAPPSAVIAAMEARGENITHAYGMTE
SYGPSTLCLWQPGVDELPLEARAQFMSRQGVAHPLLEEATVLDTDTGRPVPADGLTLGELVVRGNT
VMKGYLHNPEATRAALANGWLHTGDLAVLHLDGYVEIKDRAKDIIISGGENISSLEIEEVLYQHPE
VVEAAVVARPDSRWGETPHAFVTLRADALASGDDLVRWCRERLAHFKAPRHVSLVDLPKTATGKIQ
KFVLREWARQQEAQIADAEH
SEQ ID NO: 3
Rhodopseudomonas palustris rpDcaA (ACS3)
MSFAYFDWIAHHAEVRPERIAVVDLASSRKISYRAMDERVDRLAAHLAALGVGRGDRVAVLALNAV
ETLEVQFACFRLGAIFVPLNVRLTVHELSYIVGDAAPRVLAHDDELAPMAKELKAACSVPHLLAFG
AAYEAALAASPRLGASEPVTLDDVSTIMYTSGTTGKPKGAMITHGMTFINAVNLGIPAFISQRTVE
LCVLPLFHTGGLNCYTNPVLHAGGTTLLMRAFDPGAALSIIGDPSVGLTHEFGVPSIYQFMCQHPA
FAATDLSRLQIAGVGGAPMPVPLLKIWQERGCALVQGYGMTETSPAVLMLDADDAARKAGSAGKPV
LHADLKIVGPDGDPVKPGEMGELWVKGPNITPGYWNRPDANRTSFTDGWLHTGDAARVDDEGFYYI
VDRTKDMYISGGENVYPAEVEDVLYQLPEIAEAAVIGAPDPQWGETGVAVVALKPGQELSEAKLLA
HCRERLARFKCPQRVSFVEALPRNATGKVHKPTLRERILVRETADA
SEQ ID NO: 4
Rhodopseudomonas palustris rpDcaC (ACS4)
MTSLEATGGVPGPGRIGRVAIGDILRKSARRFPDRVALTDGGRSVTYTELERDANRFANALVARGL
KPGAKISTVCNNSIEFVKALFGIHRAGLVWVPINTMLGPDDMGYILDHAGVKVAVIDDNLHGQPER
RAALEARGIDLIAINLAGKAADTGLPVEDQLIEGLSEIEPDVAFDDRDLAMIIYTSGTTSRPKGAM
HCHLAVTMAVMSNAIEMQLSRKDGITGQFPLFHCAAHVLLLSYLIVGGQMAIMRGEDPVACMEAIQ
RNKLTVFIGLPLMYQVILDHPRRKEFDLSSLRCCIYTMAPMPRPLLERAIAELCPTFVQPSGQTEM
YPATTMSQPDRQLARFGNYWGESTLVNETAIMDDAGNLLPPGEVGEIVHRGPNVMLGYYKDPEATE
AARKFGWHHTGDLALIDEHGEVLFLDRKKDMIKSGGENVASIKIEETLLAHPSVMNAAVVGLPHPQ
WGEAVSGFVKLKPGASATEAEIVEHCKKHLGGFQVPKLLRIVDEMPMTATGKLRKVELRNQFTDHF
MLGQTG
SEQ ID NO: 5
Cannabis sativa csAAE1 (ACS5)
MGKNYKSLDSVVASDFIALGITSEVAETLHGRLAEIVCNYGAATPQTWINIANHILSPDLPESLHQ
MLFYGCYKDFGPAPPAWIPDPEKVKSTNMGALLEKRGKEFLGVKYKDPISSFSHFQEFSVRNPEVY
WRTVLMDEMKISFSKDPECILRRDDDINNPGGSEWLPGGYLNSAKNCLNVNSNKKLNDTMIVWRDE
GNDDLPLNKLTLDQLRKRVWLVGYALEEMGLEKGCAIAIDMPMHVDAVVIYLAIVLAGYVVVSIAD
SFSAPEISTRLRLSKAKAIFTQDHIIRGKKRIPLYSRVVEAKSPMAIVIPCSGSNIGAELRDGDIS
WDYFLERAKEFKNCEFTAREQPVDAYTNILESSGTTGEPKAIPWTQATPLKAAADGWSHLDIRKGD
VIVWPTNLGWMMGPWLVYASLLNGASIALYNGSPLVSGFAKFVQDAKVTMLGVVPSIVRSWKSTNC
VSGYDWSTIRCFSSSGEASNVDEYLWLMGRANYKPVIEMCGGTEIGGAFSAGSFLQAQSLSSFSSQ
CMGCTLYILDKNGYPMPKNKPGIGELALGPVMEGASKTLLNGNHHDVYFKGMPTLNGEVLRRHGDI
FELTSNGYYHAHGRADDTMNIGGIKISSIEIERVCNEVDDRVFETTAIGVPPLGGGPEQLVIFFVL
KDSNDTTIDLNQLRLSENLGLQKKLNPLFKVTRVVPLSSLPRTATNKIMRRVLRQQFSHFE
SEQ ID NO: 6
Arabidopsis thaliana atACS (ACS6) (engineered)
MASEENDLVFPSKEFSGQALVSSPQQYMEMHKRSMDDPAAFWSDIASEFYWKQKWGDQVFSENLDV
RKGPISIEWEKGGITNICYNCLDKNVEAGLGDKTAIHWEGNELGVDASLTYSELLQRVCQLANYLK
DNGVKKGDAVVIYLPMLMELPIAMLACARIGAVHSVVFAGFSADSLAQRIVDCKPNVILTCNAVKR
GPKTINLKAIVDAALDQSSKDGVSVGICLTYDNSLATTRENTKWQNGRDVWWQDVISQYPTSCEVE
WVDAEDPLFLLYTSGSTGKPKGVLHTTGGYMIYTATTFKYAFDYKSTDVYWCTADCGWIGGHSYVT
YGPMLNGATVVVFEGAPNYPDPGRCWDIVDKYKVSIFYTAPTLVRSLMRDDDKFVTRHSRKSLRVL
GSAGEPINPSAWRWFFNVVGDSRCPISDTWGQTETGGFMITPLPGAWPQKPGSATFPFFGVQPVIV
DEKGNEIEGECSGYLCVKGSWPGAFRTLFGDHERYETTYFKPFAGYYFSGDGCSRDKDGYYWLTGR
VDDVINVSGHRIGTAEVESALVLHPQCAEAAVVGIEHEVKGQGIYAFVTLLEGVPYSEELRKSLVL
MVRNQIGAFAAPDRIHWAPGLPKTRSGKIMRRILRKIASRQLEELGDTSTLADPSVVDQLIALADV
SEQ ID NO: 7
Saccharomyces cerevisiae scFAA2 (ACS7)
MAAPDYALTDLIESDPRFESLKTRLAGYTKGSDEYIEELYSQLPLTSYPRYKTELKKQAVAISNPD
NEAGFSSIYRSSLSSENLVSCVDKNLRTAYDHFMESARRWPQRDCLGSRPIDKATGTWEETFRFES
YSTVSKRCHNIGSGILSLVNTKRKRPLEANDFVVAILSHNNPEWILTDLACQAYSLTNTALYETLG
PNTSEYILNLTEAPILIFAKSNMYHVLKMVPDMKFVNTLVCMDELTHDELRMLNESLLPVKCNSLN
EKITFFSLEQVEQVGCENKIPAIPPTPDSLYTISFTSGTTGLPKGVEMSHRNIASGIAFAFSTFRI
PPDKRNQQLYDMCFLPLAHIFERMVIAYDLAIGFGIGFLHKPDPTVLVEDLKILKPYAVALVPRIL
TREEAGIKNALDKSTVQRNVANTILDSKSARFTARGGPDKSIMNELVYHRVLIDKIRDSLGLSNNS
FIITGSAPISKDTLLELRSALDIGIRQGYGLTETFAGVCLSEPFEKDVGSCGAIGISAECRLKSVP
EMGYHADKDLKGELQIRGPQVFERYFKNPNETSKAVDQDGWESTGDVAFIDGKGRISVIDRVKNFF
KLAHGEYIAPEKIENIYLSSCPYITQIFVFGDPLKTFLVGIVGVDVDAAQPILAAKHPEVKTWTKE
VLVENLNRNKKLRKEFLNKINKCTDGLQGFEKLHNIKVGLEPLTLEDDVVTPTFKIKRAKASKFFK
DTLDQLYAEGSLVKTEKL
Short chain acyl-CoA oxidases/dehydrogenases (AOX)
SEQ ID NO: 8
Geobacillus thermoleovorans AOX1
MENGDHAGTGDEDFGYIGKRGNETMNFRLSEEHEMLRKMVREFAENEVAPTAAERDEEERFDRGIF
NKMAELGLTGIPWPEEYGGIGSDYLAYVIAVEELSRVCASTGVTLSAHISLASWPIYKEGNEEQKQ
KYLRALATGEKLGAYALSEPGAGSDVASMKTRAVKDGDHYILNGSKVWITNGGEAEIYVVFAVTDP
EKRHKGISAFIVEKGTPGFSFGKKEKKLGIRSSPTTELIFEDCRIPKENLLGQEGEGFKIAMMTLD
GGRNGIAAQAVGIAQGALDAAVDYAKQRVQFGKPIIEQQGVAFKLADMATAIEAARLLTYQAAWLE
SNGLPYGKASAMAKLFAGDTAMKVTVEAVQIFGGNGYTKDYPVERFMRDAKITQIYEGTQEIQRLV
ISRMLTRN
SEQ ID NO: 9
Geobacillus thermoleovorans AOX2
MKNKQLHRGMETMMNEDFTPEQEMLRQTVRKFVDKEIMPYIKEWDERGEFDRNIFKRLAELNLMGV
CIPEEYGGMGMDYNSLAIVCEELERGDTAFRTAVSVHTGLNSLTLLQWGTEEQKQKYLVPQARGEK
IGAFGLTEPNAGSDVASIQTTAVRDGDDYILNGQKTWISLADIADHFLVFAYTDKSKKHRGISAFI
VERTMPGFSSRPIKGKLGIRSGNTGELFFDNVRVPKENLLGEEGEGFKIAMSALDNGRETVAAGAV
GLIMACLEASVKYCHERKTFGKEIGRHQLVQQMIARMEAGLQISRLLVYKVGELKNEGRRCTRETS
LAKWIACDYANQAADDAVQIHGAYGYSNEYPVERYLRNSKAPVIYEGTREIHTIMQAEYVLGYRQD
KPLRKTLPAWRPAEN
SEQ ID NO: 10
Geobacillus thermoleovorans AOX3
MSATSEGGHFMEFRFTEEQEMMRQMVREFAAAEIAPFVERMEQGEFPRPILAKMAELGLMGITVPE
QYGGAGMDFVSYIIAIHEISKVSPTVGVILSVHTSVGTNPILYFGTEEQKQKYVTKLARGEYLGAF
CLTEPSAGSDAKSLKTKAVRRGDRYVINGSKIFITNGGEADTYIVFARTNPEEAGSRGISAFIVEK
GTPGMSIGKDEKKMGLHGSRTVTITFEDAEVPAENLLGQEGEGFKIAMANLDVGRIGIAAQALGIA
EAAVEHAVAYAKERVQFGKPIIEQQGVAFKLADMATAAEAAKWLVYRAAWLRAQGLPCGKEASMAK
LFASQTAMDNAIEAVQIFGGNGYTKDYPVERLFRDAKITQIYEGTSEIQRIVISKHLCQAC
SEQ ID NO: 11
Cucurbita pepo cpAOX4 (AOX4)
MTVDTKMNREDDKDKNARSAYFGSPALDVSVAFPQATAASVFPPSVSDYYQENDLLTPEEQALRKK
VRQCMEKEIAPIMTKYWEKAEFPFHVIPKLGNLCIAGGTIKVPAFCFLFKISYNFYXSSIIALCGS
EEQKQKYLPSLAKLDTIACWALTEPENGSDASGLRTTATKVEGGWLIEGKKRWIGNSTFADLLVIF
ARNTITNEINGFIIKKNSPGLMVTKIENKIGLRIVQNGDIVMNKVFVPDEDRLPGVNSFKDTNKVL
AVSRVMVAWQPIGIAMGVYDMCHRYLKEREQFGAPLAAFQVNQQKLVLMLGNVQAMELMGWRLCKL
YEKGTMTPGQASLGKAWITLRARETVALGRELLGGNGILSDELVAKAFCDLEPIYTYEGTYDINTL
VTGREITGVASFKPASLAKRSRL
SEQ ID NO: 12
Arabidopsis thaliana atACO4 (AOX5)
MAVLSSADRASNEKKVKSSYFDLPPMEMSVAFPQATPASTFPPCTSDYYHENDLLTPEEQAIRKKV
RECMEKEVAPIMTEYWEKAEFPFHITPKLGAMGVAGGSIKGYGCPGLSITANAIATAEIARVDASC
STFILVHSSLGMLTIALCGSEAQKEKYLPSLAQLNTVACWALTEPDNGSDASGLGTTATKVEGGWK
INGQKRWIGNSTFADLLIIFARNTTTNQINGFIVKKDAPGLKATKIPNKIGLRMVQNGDILLQNVE
VPDEDRLPGVNSFQDTSKVLAVSRVMVAWQPIGISMGIYDMCHRYLKERKQFGAPLAAFQLNQQKL
VQMLGNVQAMFLMGWRLCKLYETGQMTPGQASLGKAWISSKARETASLGRELLGGNGILADELVAK
AFCDLEPIYTYEGTYDINTLVTGREVTGIASFKPATRSRL
SEQ ID NO: 13
Candida tropicalis ctAOX (AOX6)
MTFTKKNVSVSQGPDPRSSIQKERDSSKWNPQQMNYFLEGSVERSELMKALAQQMERDPILFTDGS
YYDLTKDQQRELTAVKINRIARYREQESIDTENKRLSLIGIFDPQVGTRIGVNLGLFLSCIRGNGT
TSQLNYWANEKETADVKGIYGCFGMTELAHGSNVAGLETTATFDKESDEFVINTPHIGATKWWIGG
AAHSATHCSVYARLIVDGQDYGVKTFVVPLRDSNHDLMPGVTVGDIGPKMGRDGIDNGWIQFSNVR
IPRFFMLQKFCKVSAEGEVTLPPLEQLSYSALLGGRVMMVLDSYRMLARMSTIALRYAIGRRQFKG
DNVDPNDPNALETQLIDYPLHQKRLFPYFVPPMSSPSVPSRLNTPSRPPWSNWTSPLKRTTPRLIF
KSIDDMKSLFVDSGSLKSTATWLGAEAIDQCRQACGGHGHSSYNGFGKAYNDWVVQCTWEGDNNVL
GMSVGKPIVKQVISIEDAGKTVRGSTAFLNQLKEYTGSNSSKVVLNTVADLDDIKTVIKAIEVAII
RLSQEAASIVKKESFDYVGAELVQLSKLKAHHYLLTEYIRRIDTFDQKELAPYLITLGKLYAATIV
LDRFAGVELTENVASTEAITALASVQIPKLCAEVRPNVVAYTDSFQQSDMIVNSAIGRYDGDIYEN
YFDLVKLQNPPSKTKAPYSDALEAMLNRPTLDERERFQKSDETAAILSK
SEQ ID NO: 14
Yarrowia lipolytica YLAOX3 (AOX7)
MISPNLTANVEIDGKQYNTFTEPPKALAGERAKVKFPIKDMTEFLHGGEENVTMIERLMTELERDP
VLNVSGDYDMPKEQLRETAVARIAALSGHWKKDTEKEALLRSQLHGIVDMGTRIRLGVHTGLEMGA
IRGSGTKEQYDYWVRKGAADVKGFYGCFAMTELGHGSNVAGLETTATYIQDTDEFIINTPNTGATK
WWIGGAAHSATHTACFARLLVDGKDYGVKIFVVQLRDVSSHSLMPGIALGDIGKKMGRDAIDNGWI
QFTNVRIPRQNMLMKYAKVSSTGKVSQPPLAQLTYGALIGGRVTMIADSFFVSQRFITIALRYACV
RRQFGTTPGQPETKIIDYPYHQRRLLPLLAFTYAMKMAADQSQIQYDQTTDLLQTIDPKDKGALGK
AIVDLKELFASSAGLKAFTTWTCANIIDQCRQACGGHGYSGYNGFGQAYADWVVQCTWEGDNNVLC
LSMGRGLIQSCLGHRKGKPLGSSVGYLANKGLEQATLSGRDLKDPKVLIEAWEKVANGAIQRATDK
FVELTKGGLSPDQAFEELSQQRFQCAKIHTRKHLVTAFYERINASAKADVKPYLINLANLFTLWSI
EEDSGLFLREGELQPKDIDQVTELVNHYCKEVRDQVAGYTDAFGLSDWFINAPIGNYDGDVYKHYF
AKVNQQNPAQNPRPPYYESTLRPFLFREDEDDDICELDEE
SEQ ID NO: 15
Thermobifida fusca AOX8
MDFTLTEEQTALRAMAAEFVDREITPHVVEWDRAESVPRDLVPRLGELGFLGMTIPEEYGGVGGDH
VSYALVMEELGRGDSSVRGLVSVSLGLVAKSIATYGTEEQRQRWLPGLCSGELLGCFGLTEPDTGS
DAASLSLKAVRDGDDYVLTGSKMFITNGTWADVAIIFARTGGPGPKGITAFLVPTDTPGLTRTTIH
GKLGLRGQPTAQLFLDEVRVGPDAILGEENQGFRIAMAALDKGRVSVAAGAVGAARGALESALAHA
KERVQFGRPIAGFQLVQEMLADMAVETDAARLLVWRAADLIDRGEPFSTAASMAKLYATETAVRAA
NNAMQVFGGYGYIDEYPPGKYLRDARVTTLYEGTSQIQKLIIGRALTGISAFA
SEQ ID NO: 16
Thermomonospora curvata AOX9
MDAQDFADVLAAVRSFVREQVIPREEEIEETDAIPESLRQAAREMGLFGYALPEEYGGLGLSLSEE
VRLAFELGYASPAFRSMFGTSNGIAGQVLVNAGTEEQKRQWLPRLASGEVIGAFALTEAEAGSDPS
TLSTTAVKDDDGNYVINGAKRFITNAPEADVEMVFARTGGPGSRGISTELVERDAPGLKVGPPDEK
MGQRGAHTAEVFFDNVRVPASAMVGPEGTGERTAMASLAHGRLSIAAVCVGLAQRILDETVAYAKE
RAQGGRKIGEYQLVQGMIADSQAELYAGRAMVLEAARAYDAGEDRKLGPSCAKYFCSEMLGRVADR
AVQVFGGMGYMRGVAVERFYRDARLFRIYEGTSQIQQLVIAKRLLAD
SEQ ID NO: 17
Geobacillus thermodenitrificans AOX10
MNFQLSEEHEMLRKMVREFAENEVAPTAAERDEEERFDRSIENKMAELGLTGIPWPEEYGGIGSDY
LAYVIAVEELSRVCASTGVTLSAHISLASWPIYKEGTEEQKQKYLRALATGEKLGAYGLSEPGAGS
DVASMKTRAIKDGDHYVLNGSKVWITNGGEAEIYVVFAVTDPEKRHKGISAFIVEKGTPGESIGKK
EKKLGIRSSPTTELIFEDCRIPKENLLGQEGEGFKIAMMTLDGGRNGIAAQAVGIAQGALDAAVDY
AKQRVQFGKPIAEQQGVSFKLADMATAIEAARLLTYQAAWLESNGLPYGKASAMAKLFAGDTAMKV
TVDAVQIFGGNGYTKDYPVERFMRDAKITQIYEGTQEIQRLVISRMLTRD
SEQ ID NO: 18
Caldivirga maquilingensis AOX11
MHPLLEEVHELLRRSTVEFAEARVEPLAKAIEVNGEYPRQLMGELGKQGLLAPMAPPEYGGGGLDF
RGEVIIIEEVAKHSPTLATLMEVQGALIIDSLLSHGSREVRERFLEGLVKGDLIASFALTEPCCGS
DAGAIETRAVKDGGEWVINGRKAWVTSGQYADLYLVFARTGSIEERHRAITAFLVPRGGCIEVNPM
GVMGIRGGGTAEVSLRDCRVPDEYRVGEVNRGFYLAMEKLNLGRTCVGAIGLGIAERAFTEAYDYA
QVRRIYGGTLAELGVIQGYLAQMYTQVEALRGLIYLTAYMRDKGLSEFTKYAQAAKYLGSSTAVNV
TRTAIQVMGAVGLSTESPLEYLYRDAKATEVYEGSNEVILYSLFKMLRK
SEQ ID NO: 19
Pyrobaculum aerophilum AOX12
MVFPFDTVEDYSIVLTREHEMERKAVREFVEREIAPRAMEIEETDEVPRDILKKIAENGFFGIGIP
EKYGGQGGDHRMAAILSEEFCRVLPGLSVYFGTNELFLTPIMLEGTEEQRQKYVPPIARGEKFGAF
AVTEPCCGSDVAGIQTKAEKKGDKWVINGRKAFISSSDVADFFIVLARTYPPPDKKVRYLGLTFFI
VEKDTPGFKVEQCYHKMGLYGNHACELVLENVEVPDENRVGEEGMGFLYAMETFDRTRIGVAAQAV
GMAQAAFEKAFQYVHQRQAFGVPIAYFQAIQFSLVEMMAKIFTARLLTYLAAKLADEDRREFTEVA
SLAKFYATEVAEEVISEAINLHGGVGVIRETGVERFLRSVKITQIYEGANNIQKLVAYRQLIRLLK
EKGQIPDEIARLVT
SEQ ID NO: 20
Thermoplasma acidophilum AOX13
MGNFGVTQDEELVLSFVRKFAEEELKPLAKEIDEKMEVPRKIIDRMKDLGLFATYIPKEYGGYGMS
FPFLVRAIEEISKACPSTALVLDGALTLFAEPLIMFGSEDLKKRYLPRVAAGSVGGLAITEPGAGS
DAAGISATAVKKGDRYVINGDKIFISNGRISDFFVLDAVTDPGKRHRGITAFVADRDTPGLKISRD
IHKMGIRGSSTVELAFEDMEIPAENIVGKENEGFKVIMETLDAGRIGIAAQALGIAENALAEAIDY
VKQRKQFGTEIANFEGIQFMIAEMATEIEAARYLTYVAAEKWQNKENTIEISAMAKMKASDVAMRV
TTDALQLFGGYGYTTDLDAERHMRDAKITQIYEGTNQIQRLVIAKEILKKTRYY
Enoyl-CoA isomerases
SEQ ID NO: 21
Saccharomyces cerevisiae ScECI1 (ECI1)
MSQEIRQNEKISYRIEGPFFIIHLMNPDNLNALEGEDYIYLGELLELADRNRDVYFTIIQSSGREF
SSGADEKGIAKAQGDDTNKYPSETSKWVSNFVARNVYVTDAFIKHSKVLICCLNGPAIGLSAALVA
LCDIVYSINDKVYLLYPFANLGLITEGGTTVSLPLKFGTNTTYECLMENKPFKYDIMCENGFISKN
FNMPSSNAEAFNAKVLEELREKVKGLYLPSCLGMKKLLKSNHIDAENKANSVEVNESLKYWVDGEP
LKRFRQLGSKQRKHRL
SEQ ID NO: 22
Kluyveromyces marxianus KmECI1 (ECI2)
MVSIVKSSRIDSRKQGPFLIIFLNDEKTLNSLEGNDYLYLAHLLIENDKDADTSFTVIQSSGRFFS
SGANFSSIVKENGKKNKDGELPKWAAAFLSRNTYVTTAFINHSKPLICCLNGPAVGLSAAIVMLCD
IVYVMNNKVYLQFPFAKIGLVTEGAVAVTLPLKIGYARAQNVLFENKRVTYDMLENTVSVKNYELD
DWQQFNARVLEDLGKDVKNINMASIIGMKALIKETWKGHLLQANVSEVTDAMPFWIEGIPQSNENK
LLKKAESRKNKLKPKL
SEQ ID NO: 23
Ogataea parapolymorpha OpECI1 (ECI3)
MSPSYKIVNQVFVIVLDDPHTRNALTIPQFVQLAELLELADKHPHTTATLVVGRGPMESAGANIKT
VAELKNKTSSEILSEISAKNLYLVHLFTSHRKLLVVGLNGPVIGLTASLVALADLVYAQNQKVEMS
FPFTNIGLTTECAAAVSLPHRLGLSTALEHVLLAKPLSAAKLHELGLVNRVEDLDDCDAENDQLAN
MLTKELVGLDRDTIFVNKRLMRHTFDTQVRAQALQETMSGVADWTANKPQTAFDEIVAGKRKHKL
SEQ ID NO: 24
Chaetomium thermophilum CtECI1 (ECI4)
MSDPAIKIEYRGRLAIVTINNEKKLNALNGNQYYALAQALREVATHDEVYITLLIGKGRYFSAGAD
VSLSQPSPEEQRIRSENPHRFWLQSFVANNLNITHAFYTHPKILVVGLNGPVIGLSAALVAFADFI
YATPQTELLTPFSSLGLVTEGGACRALIQRLGPARANEALIMSKRITAEELERAGFVNKIFSEVGK
GEDEKFKQLVLNEINERLGEHLVGDSLLGIKKLIRRPETQILDAANVAEVFAGLDREVSGVPQGEF
EKIATGKKRHKL
SEQ ID NO: 25
Scheffersomyces stipitis SsECI1 (ECI5)
MEGEDILYEVRGKVTIITENIPQKLNALNGEQYLLLAKLVERADKEEDTILTLIQSSGREFSAGAN
FADKSLANTDAADLESHEYWLNRFVARNTYLTELFHNHRKILAAAVNGPVIGLSASLLALCDLIYV
KEEKDFYILTPFANLGLVAEGAASATLFLRLGWSKASEALLLAKPISGKDLNNLGFINKTYDGQFK
TTEEFNQAVHDELVNAFENLHEDSIIQNKQLLKSNRDQLITSANSKEVIRGENKWIEGVPQGREVQ
LAQKDIKHKF
SEQ ID NO: 26
Thermomonospora curvata TcuEchA5 (ECI6)
MAVRIEDRGHVRIVTLDRPERRNAVDGPMADRIAEVFAAFERDGRARVAVLTGAGGTFCAGNDLKA
IAAGEFPVPTRQAPPMRVTRAAPAKPVIAAIEGPCFGGGLELALWCDLRVASATAEFGLLNLVHGL
PSMDAGTVRLPRLIGHARALEMIVTARRVPAAEAMGWGLLNRLVEPGRALDEALRLAEAVAALPQE
PMLASRRSALQQWGRPEAEAFGREVELALAALGNPG
SEQ ID NO: 27
Thermomonospora curvata TcuEchA10 (ECI7)
MERLPISTAHCTVEQDGHVVIVTMNRPEARNALSTSMLVGMADAFAYISQTPEVRVGILTGAGGHE
CSGADLKAMGTPPEDEREQRRAAEITNYHWKGLLREAVPTKPIIAAIEGYAVAGGTELLLGTDLRV
VAEGATLGLYEAKRGLFPMGGSVVRLPRQIGYAPAMEILLTARSVTPQEALAMGLINRVVPDGQAL
SAARELADQIAACAPLSVQAILRAHRETFHLPEEEALKVSDEIGWPIFATEDAQEGPRAFREKRPP
VFKGR
SEQ ID NO: 28
Thermomonospora catenispora TcaEchA5 (ECI8)
MSGVRVERSGPVTTVILARPEVRNAIDGPTAAALAEAFRAFDADPDAAVAVLWGEGGTFCAGADLK
AIGTERGNRVDPPPADAPLGCTRMRLSKPVIAAISGHAVAGGLELALWADLRVAEPDAVEGVFCRR
WGVPLVDGGTVRLPRLIGMSRAMDMILTGRPVDAREAYDIGLVNRLVEPGRARAAAEELAADLARF
PQTCLRGDRLSLLEQDGMTEDEALANEYRRGVNALGEAIEGAARFAAGAGRHGDFTDI
SEQ ID NO: 29
Thermomonospora catenispora TcaEchA10 (ECI9)
MASDTDERVLLDRREGILVITLNRPERLNAVTADVSELMLDVLNGLMRDSRTHVVVLTGAGRGFCS
GMDISGDDAIAAGASSEGRPQALYRGIALSGEVTARLREIPQPVIGALHGPVAGMGASWALACDLR
VADPTTTLVLPFTNLGLSGGDCGLSWLLPRLVGPTKAADLLYRSQRLSFEQIRELGLVTEEADRGG
DLDRALELADELLQRSPYGLRRTKELLNASLDAPGLRQQLLAETGVQTLAFFTQDLAEGMTAALER
RPPREANR
SEQ ID NO: 30
Arabidopsis thaliana AtECI1 (ECI10)
MCSLEKRDRLFILKLTGDGEHRLNPTLLDSLRSTINQIRSDPSFSQSVLITTSDGKFFSNGYDLAL
AESNPSLSVVMDAKLRSLVADLISLPMPTIAAVTGHASAAGCILAMSHDYVLMRRDRGFLYMSELD
IELIVPAWFMAVIRGKIGSPAARRDVMLTAAKVTADVGVKMGIVDSAYGSAAETVEAAIKLGEEIV
QRGGDGHVYGKMRESLLREVLIHTIGEYESGSSVVRSTGSKL
SEQ ID NO: 31
Arabidopsis thaliana AtECI3 (ECI11)
MCTLEKRGDLFLLTLTGEDEHRFHPDTIASVLSLLEQAKSQSTKGSVLITTGHGKFFSNGEDLAWA
QSAGHGAIKRMHQMVKSFKPVLAALLDLPMPTIAALNGHAAASGLMFALSHDYVEMRKDRGVLYMS
EVDIGLPVPDYFSALVVAKVGSGIARRELLLSGKKLKGEEAVALGIVDSAAHDSAEGVVEATVSLG
ESLAAKKWNGEVYATIRKSLYPELCRMVDLTANNLATHNL
SEQ ID NO: 32
Jatropha curcas JcECI2 (ECI12)
MCTLEKRDDIFILTFTGADEHRLSPTLIDSVLSALREVKSQATRGSVLITTSQGKFFSNGEDLAWA
RAAGSNSKAMERLRFMVQSFKPVVAEMISLPMPTVAALQGHAAAAGELFSICHDYVLMRSDKGVLY
MSEVDLGLPLPDYFASVERDKLHSVSARRDVLLRGAKVKGEEAVRMGIVEAAYDSEAKLTDATIRL
GEQLASRKWKGDVYKEIRKSLYPDICGVLALDEENIVSKL
SEQ ID NO: 33
Citrus clementina CcECI2 (ECI13)
MCTLEKHGDVFVLTLTGSSNVDEHRLGPTAIDSILSAIAQAKAEATPGSALITTSHGKFFSNGFDL
AWAQAAGSRTGARERLHYMFKSFRPLVAAMMDLPMPTVAAVNGHAAAAGFILALSHDYVVMRRDKG
VLYMSEVDIGLTLPDYCAALFREKVGSATARRDVLLRAKKIKGEEALRMGLVEAAYDSEERVAEAS
MRLGKQLAGRKWAGEVYAEIRKSLYPDLCGVLGLDIKAVESNSKL
SEQ ID NO: 34
Phoenix dactylifera PdECI1 (ECI14)
MCTLEKRGRVYLLTLTGDGEHRLNPTLLNAIRSALARVRSDSARAGGGSALVTAAEGKYFSNGFDL
AWAKSSPANRTTIMTSAFQHVVADLMTLPMPTIAAVTGHASAAGFALALIHDYVVMRRDRGFLYMS
ELDIEIPIAEFVMALFRSKIADPRVRRDVLLRAPKITAAEAERKGIIERAVGGAAEAVETAVRMGE
ELAEKNWNGEVYASIRNAAFPEVCRALELAEEGDEETKRLASKL
SEQ ID NO: 35
Phoenix dactylifera PdECI2 (ECI15)
MCSLEKRGRVFVLTLTGDGEHRLGHDLIAAVRSALARVRAESAAGPGGYAFVTAAEGRFFSNGFDL
AWANSAGSRSAARERLSSLVAVERPIVADLMSLPMPTIAAVTGHAAAAGFMLAICHDYVAMRGDRG
FLYMSELDIGLPFPPYFMALMRAKIADPRALRDVVLRAAKIPAADAKEKGIIDRVHGGAAETVEVA
VRMGEELAARKWNGSIYASIRMAAFPELCRAVGLAEEGDEEKPKTVAAKL
SEQ ID NO: 73
Escherichia coli EcPaaG (ECI16)
MMEFILSHVEKGVMTLTLNRPERLNSFNDEMHAQLAECLKQVERDDTIRCLLLTGAGRGFCAGQDL
NDRNVDPTGPAPDLGMSVERFYNPLVRRLAKLPKPVICAVNGVAAGAGATLALGGDIVIAARSAKE
VMAFSKLGLIPDCGGTWLLPRVAGRARAMGLALLGNQLSAEQAHEWGMIWQVVDDETLADTAQQLA
RHLATQPTFGLGLIKQAINSAETNTLDTQLDLERDYQRLAGRSADYREGVSAFLAKRSPQFTGK
SEQ ID NO: 74
Escherichia coli EcCaiD (ECI17)
MKQQGTTLPANNHTLKQYAFFAGMLSSLKKQKWRKGMSESLHLTRNGSILEITLDRPKANAIDAKT
SFEMGEVELNERDDPQLRVAIITGAGEKFFSAGWDLKAAAEGEAPDADFGPGGFAGLTEIFNLDKP
VIAAVNGYAFGGGFELALAADFIVCADNASFALPEAKLGIVPDSGGVLRLPKILPPAIVNEMVMTG
RRMGAEEALRWGIVNRVVSQAELMDNARELAQQLVNSAPLAIAALKEIYRTTSEMPVEEAYRYIRS
GVLKHYPSVLHSEDAIEGPLAFAEKRDPVWKGR
SEQ ID NO: 75
Escherichia coli EcPaaF (ECI18)
MSELIVSRQQRVLLLTLNRPAARNALNNALLMQLVNELEAAATDTSISVCVITGNARFFAAGADLN
EMAEKDLAATLNDTRPQLWARLQAFNKPLIAAVNGYALGAGCELALLCDVVVAGENARFGLPEITL
GIMPGAGGTQRLIRSVGKSLASKMVLSGESITAQQAQQAGLVSDVFPSDLTLEYALQLASKMARHS
PLALQAAKQALRQSQEVALQAGLAQERQLFTLLAATEDRHEGISAFLQKRTPDEKGR
SEQ ID NO: 76
Escherichia coli EcMenB (ECI19)
MIYPDEAMLYAPVEWHDCSEGFEDIRYEKSTDGIAKITINRPQVRNAFRPLTVKEMIQALADARYD
DNIGVIILTGAGDKAFCSGGDQKVRGDYGGYKDDSGVHHLNVLDFQRQIRTCPKPVVAMVAGYSIG
GGHVLHMMCDLTIAADNAIFGQTGPKVGSFDGGWGASYMARIVGQKKAREIWFLCRQYDAKQALDM
GLVNTVVPLADLEKETVRWCREMLQNSPMALRCLKAALNADCDGQAGLQELAGNATMLFYMTEEGQ
EGRNAFNQKRQPDFSKFKRNP
SEQ ID NO: 77
Escherichia coli EcFadB (ECI20)
MLYKGDTLYLDWLEDGIAELVEDAPGSVNKLDTATVASLGEAIGVLEQQSDLKGLLLRSNKAAFIV
GADITEFLSLFLVPEEQLSQWLHFANSVENRLEDLPVPTIAAVNGYALGGGCECVLATDYRLATPD
LRIGLPETKLGIMPGFGGSVRMPRMLGADSALEIIAAGKDVGADQALKIGLVDGVVKAEKLVEGAK
AVLRQAINGDLDWKAKRQPKLEPLKLSKIEATMSFTIAKGMVAQTAGKHYPAPITAVKTIEAAARF
GREEALNLENKSFVPLAHTNEARALVGIFLNDQYVKGKAKKLTKDVETPKQAAVLGAGIMGGGIAY
QSAWKGVPVVMKDINDKSLTLGMTEAAKLLNKQLERGKIDGLKLAGVISTIHPTLDYAGEDRVDIV
VEAVVENPKVKKAVLAETEQKVRQDTVLASNTSTIPISELANALERPENFCGMHFENPVHRMPLVE
IIRGEKSSDETIAKVVAWASKMGKTPIVVNDCPGFFVNRVLFPYFAGFSQLLRDGADERKIDKVME
KQFGWPMGPAYLLDVVGIDTAHHAQAVMAAGFPQRMQKDYRDAIDALFDANRFGQKNGLGEWRYKE
DSKGKPKKEEDAAVEDLLAEVSQPKRDESEEEIIARMMIPMVNEVVRCLEEGIIATPAEADMALVY
GLGFPPFHGGAFRWLDTLGSAKYLDMAQQYQHLGPLYEVPEGLRNKARHNEPYYPPVEPARPVGDL
KTA
Fatty acyl-CoA reductases (EC 1.2. 1.84)
SEQ ID NO: 36
Lachnoclostridium phytofermentans LpEutE (FAR1)
MTVNEQLVQDIIKNVVASMQLTQTNKTELGVEDDMNQAIEAAKEAQLVVKKMSMDQREKIISAIRK
KTIEHAETLARMAVEETGMGNVGHKILKHQLVAEKTPGTEDITTTAWSGDRGLTLVEMGPFGVIGA
ITPCTNPSETIICNTIGMLAGGNTVVENPHPAAIKTSNFAVQLINEASLSAGGPVNIACSVRKPTL
DSSKIMMSHQDIPLIAATGGPGVVTAVLQSGKRGIGAGAGNPPVLVDETADIRKAAEDIINGCTED
NNLPCIAEKEVVAIDAIANELMNYMVKEQGCYAITKEQQEKLTNLVITPKGLNRNCVGKDARTLLG
MIGIDVPSNIRCIIFEGEKEHPLISEELMMPILGIVRAKSEDDAVEKAVWLEHGNRHSAHIHSKNV
DRITTYAKAIDTAILVKNAPSYAAIGFGGEGFCTFTIASRTGEGLTSASTFTKRRRCVMSDSLCIR
SEQ ID NO: 37
Thermoanaerobacterium saccharolyticum TsEutE (FAR2)
MKVKEEDIEAIVKKVLSEFNFEKNTKSFRDFGVFQDMNDAIRAAKDAQKKLRNMSMESREKIIQNI
RKKIMENKKILAEMGVSETGMGKVEHKIIKHELVALKTPGTEDIVTTAWSGDKGLTLVEMGPFGVI
GTITPSTNPSETVLCNSIGMIAAGNSVVENPHPGAVNVSNYAVKLVNEAVMEAGGPENLVASVEKP
TLETGNIMFKSPDVSLLVATGGPGVVTSVLSSGKRAIGAGAGNPPVVVDETADIKKAAKDIVDGAT
FDNNLPCIAEKEVVSVDKITDELIYYMQQNGCYKIEGREIEKLIELVLDHKGGKITLNRKWVGKDA
HLILKAIGIDADESVRCIIFEAEKDNPLVVEELMMPILGIVRAKNVDEAIMIATELEHGNRHSAHM
HSKNVDNLTKFGKIIDTAIFVKNAPSYAALGYGGEGYCTFTIASRTGEGLTSARTFTKSRRCVLAD
GLSIR
SEQ ID NO: 38
Caldanaerobius polysaccharolyticus CpEutE (FAR3)
MAGIREEDIELIVRRVLSNLDLKNLKAAVKKDIGVFEDMKQAISAAKKAQKELKSMSIEFREKIIQ
NIRKKTLENARIMAEMGVQETGMGKVEHKVLKHELVARKTPGTEDIITTAWSGDKGLTLVEMGPWG
VIGAITPSTNPSETVICNSIGMIAAGNSVVENPHPGAVGVSNYAVRLINEAVVEAGGPPNLAVSVA
KPTLETAEIMFKHPDINLLVATGGPGVVTAVLSTGKRAIGAGAGNPPVVVDETADIRKAAKDIVDG
ATFDNNLPCIAEKEVIAVNKVADELIYYMKQNGCYMASKEEIEELKAMVLQTRDGKYYLNRKWVGK
DASTLLKGIGVDVDDKVRCIIFEATKDHPFVVEELMMPILGIIRAENVDEAIAIAVELEHGFRHSA
HMHSKNVDNLTKFARAIDTAIFVKNAPSYAAIGFGGEGYCTFTIASRTGEGLTSARTFTKSRRCVL
ADGLSIR
SEQ ID NO: 39
Clostridium beijerinckii CbALD (FAR4)
MNKDTLIPTTKDLKLKTNVENINLKNYKDNSSCFGVFENVENAINSAVHAQKILSLHYTKEQREKI
ITEIRKAALENKEVLATMILEETHMGRYEDKILKHELVAKYTPGTEDLTTTAWSGDNGLTVVEMSP
YGVIGAITPSTNPTETVICNSIGMIAAGNAVVENGHPGAKKCVAFAIEMINKAIISCGGPENLVTT
IKNPTMESLDAIIKHPLIKLLCGTGGPGMVKTLLNSGKKAIGAGAGNPPVIVDDTADIEKAGKSII
EGCSFDNNLPCIAEKEVFVFENVADDLISNMLKNNAVIINEDQVSKLIDLVLQKNNETQEYFINKK
WVGKDAKLESDEIDVESPSNIKCIVCEVNANHPFVMTELMMPILPIVRVKDIDEAVKYTKIAEQNR
KHSAYIYSKNIDNLNRFEREIDTTIFVKNAKSFAGVGYEAEGFTTFTIAGSTGEGITSARNFTRQR
RCVLAG
SEQ ID NO: 40
Clostridium kluyveri CkALD (FAR5)
MEIMDKDLQSIQEVRTLIAKAKKAQAEFKNFSQEAVNKVIEKIAKATEVEAVKLAKLAYEDTGYGK
WEDKVIKNKESSIVVYNYIKDLKTVGILKEDKEKKLIDIAVPLGVIAGLIPSTNPTSTAIFKVLIA
LKAGNAIVESPHPTAVRSITETVKIMQKAAVEAGAPDGLIQCMSILTVEGTAELMKNKDTALILAT
GGEGMVRAAYSSGTPAIGVGPGNGPCFIERTADIPTAVRKVIGSDTEDNGVICASEQSIIAETVKK
AEIIEEFKRQKGYFLNAEESEKVGKILLRANGTPNPAIVGKDVQALAKLAGISIPSDAVILLSEQT
DVSPKNPYAKEKLAPVLAFYTVEDWHEACEKSLALLHNQGSGHTLIIHSQNEEIIREFALKKPVSR
ILVNSPGSLGGIGGATNLVPSLTLGCGAVGGSATSDNVGPENLENIRKVAYGTTTVEEIREAFGVG
AASSSAPAEPEDNEDVQAIVKAIMAKLNL
SEQ ID NO: 41
Escherichia coli EcMphF (FAR6)
MSKRKVAIIGSGNIGTDLMIKILRHGQHLEMAVMVGIDPQSDGLARARRMGVATTHEGVIGLMNMP
EFADIDIVEDATSAGAHVKNDAALREAKPDIRLIDLTPAAIGPYCVPVVNLEANVDQLNVNMVTCG
GQATIPMVAAVSRVARVHYAEIIASIASKSAGPGTRANIDEFTETTSRAIEVVGGAAKGKAIIVLN
PAEPPLMMRDTVYVLSDEASQDDIEASINEMAEAVQAYVPGYRLKQRVQFEVIPQDKPVNLPGVGQ
FSGLKTAVWLEVEGAAHYLPAYAGNLDIMTSSALATAEKMAQSLARKAGEAA
SEQ ID NO: 42
Marinobacter hydrocarbonoclasticus MhFAR2 (FAR7)
MNYFLTGGTGFIGRELVEKLLARGGTVYVLVREQSQDKLERLRERWGADDKQVKAVIGDLTSKNLG
IDAKTLKSLKGNIDHVFHLAAVYDMGADEEAQAATNIEGTRAAVQAAEAMGAKHFHHVSSIAAAGL
FKGIFREDMFEEAEKLDHPYLRTKHESEKVVREECKVPFRIYRPGMVIGHSETGEMDKVDGPYYFF
KMIQKIRHALPQWVPTIGIEGGRLNIVPVDFVVDALDHIAHLEGEDGNCFHLVDSDPYKVGEILNI
FCEAGHAPRMGMRIDSRMFGFIPPFIRQSIKNLPPVKRITGALLDDMGIPPSVMSFINYPTREDTR
ELERVLKGTDIEVPRLPSYAPVIWDYWERNLDPDLFKDRTLKGTVEGKVCVVTGATSGIGLATAEK
LAEAGAILVIGARTKETLDEVAASLEAKGGNVHAYQCDESDMDDCDRFVKTVLDNHGHVDVLVNNA
GRSIRRSLALSFDRFHDFERTMQLNYFGSVRLIMGFAPAMLERRRGHVVNISSIGVLTNAPRFSAY
VSSKSALDAFSRCAAAEWSDRNVTFTTINMPLVKTPMIAPTKIYDSVPTLTPDEAAQMVADAIVYR
PKRIATRLGVFAQVLHALAPKMGEIIMNTGYRMFPDSPAAAGSKSGEKPKVSTEQVAFAAIMRGIY
W
SEQ ID NO: 43
Vulcaniibacterium thermophilum VtFAR2 (FAR8)
MGYFVTGATGFIGRHLVSRLFERKGTVYVLVRKESQKKLDDIARRMGWDTKRVMAVAGDMTRANCG
LSAAQMRSLKGKVRHFFHLAAIYDLTADADAQYAANVEGTRHALDLAAALGAGCFHHASSIAAAGL
YPGVFREDMFEEAEGLDDPYLRTKHESEGLVRKEKRLKWRIYRPGIVVGHSRTGEIDKIDGPYYFF
TFIKKMRAMLPQWMPTLGIEGGRINLVPVDYVADAMDYIAHKPKLDGHTFHLVDPAPQRVGEVLNT
FARAAHAPEMTMRLDARMFAFLPSAVRLAVGNLPPVKRMIAMLLRDEGIPKQALKFITYPTREDNR
ETERALRGSGIAVPPLESYAWRLWDYWERHLDPDLFVDRSLKSRVKGKVVVITGGSSGIGLAAARK
IAEAGAITVLCARGEDELFKVRDELRAAGGKVFAYTADLSDMADCDRFVQTVLKEHGAVDILVNNA
GRSIRRSIELSFDRFHDFERTMQLNYFGSLRLIMGFLPGMIERRRGHIINISSIGVLANSPRESAY
VASKAALDAFSRCAQGELSGKGISFTTINMPLVKTPMIAPTKIYEAVPTLTPEEAADLVVRGIIEK
PSRISTRLGTFAAVVNALAPKAYEVIMNTAFELFPDSAAAKGGGKEAQPSNEQIAFAALMRGVHW
SEQ ID NO: 44
Thermomonas hydrothermalis ThFAR2 (FAR9)
MAYLVTGGTGFIGRFLIDNLLKRKGTIHVLVRKASLKKFEALAKQRGWDRKRVVPLVGDMAAPGCG
VSAAQVRALAGKIRHVFHLAALYDLTASADQMYQANVEGTRHALDLAAALKAGCFHHVSSIAAAGL
YPGVFREDMFEEAEGLDDPYLRTKHESEGLVRHETRIKWRIYRPGMVVGHSQTGEIDKIDGPYYFF
TLIKKMRQMLPPWMPMLGLEGGRINIVPVDFVADALDHIAHKPRLDGHCFHLTDPDPLRVGEVLNV
FARAAHAPEMSMRLDARMFAFVPPSVRMAVGSLPPVRRIVGALLRDFRIPRQVLKFITYPTREDNR
EAERALKGSGIAVPRLEDYAWRLWDYWERHLDPDLFIDRSLKGTVAGKVVVITGGSSGIGLATARK
VAAAGAITIIVARGQEELFKARDEMQAAGGKVFAYTADLSDMADCDRLVAEVLKAHGHVDILINNA
GRSIRRSIELSYDRFHDFERTMQLNYFGSLRLIMGELPTMVQRRKGHIINISSIGVLANSPRESAY
VASKAALDAFSRCAQGELSGKGIHFTTINMPLVKTPMIAPTKVYENVPTLTPDEAADLVVKGIIEK
PSRIATRLGIFAAVINAVAPRAYEAVMNTAFELFPDSAAAKGDKQALRDETPSQEQIAFAALMRGV
HW
SEQ ID NO: 45
Heliothis virescens HvFAR (FAR10)
MVVLTSKETKPSVAEFYAGKSVFITGGTGELGKVFIEKLLYSCPDIVNIYMLIREKKGLSVSERIK
QFLDDPLFTRLKDKRPADLEKIVLIPGDITAPDLGITAANEKMLIEKVSVIIHSAATVKENEPLPT
AWKINVEGTRMMLALSRRMKRIEVFIHISTAYTNTNREVVDEILYPAPADIDQVYQYVKEGISEED
TEKILNGRPNTYTFTKALTEHLVAENQAYVPTIIVRPSVVAAIKDEPLKGWLGNWFGATGLTVFTA
KGLNRVIYGHSNYIVDLIPVDYVANLVIAAGAKSNTSSELKVYNCCSSSCNPVKIGTLMSMFADDA
IKQKSYAMPLPGWYIFTKYKWLVLLLTELFQVIPAYITDLSRHLVGKSPRYIKLQSLVNQTRSSID
FFTNHSWVMKADRVRELYASLSPADKYLFPCDPVNINWTQYLQDYCWGVRNFLEKKT
SEQ ID NO: 46
Melipona quadrifasciata MqFAR (FAR11)
MSTISSAQNTSVKDFYRDRCVFVTGVTGFMGKVLVEKLLRSCPGLKTIYVLIRNKKGQDAHQRLRA
LLNGPLEDKLRRDAPNELQKVIAVPGDITEHELGISESDQNILIRNVSVVFHSAATVKFDEALKIS
VTINMIGTKQLLNLCHRMQNLEALIHVSTAYCNCDRTDIAEQIYPLIAEPEEIFALTKVMDDKMID
DITPILIGKRPNTYTFTKALAERMLETESDYLPIAIVRPTIVLSSYKEPVAGWLDNWNGPTGIIAA
AGKGFFRSMLCRDDMIADLVPVDIVINLMIVAAWKTATNRTKTIPIYNCCTGQQNPITWKKFVDLT
FKYSRLHPYNDVIWYPGGNCHTSTIINKICMLLQHIVPAHILDFTHRLKGKTANMVTLQTKLEKAT
KYLEYFTMQQWSERDDNVRELNEQLSPEDRQIFAFDVRQIDWSLYLEHYILGIRHELLKENPDTLP
AARVHLRKLYWFHKAVQFGVLLVLLRFLLLRSTVTQNACYSLVSLVLRMCRIIV
SEQ ID NO: 52
Luteitalea pratensis LupFAR (FAR12)
MADVPAAKPHSDRDLASMAEARTLARAARVAQAQLAELPQDRIDAIVDAMAAAVRQEVDALARLAC
EETGFGVYADKIIKNRFAAEQVHEFIRPMRTVGVLRRDETRKIIEIAEPFGVVAAIVPSTNPTSTA
IYKILIALKARCAIVISPHPSAARCISRTAEIMAAAAARAGAPADVIGWMSVVTLEGTQELMRQRE
VAVILATGGLGLVRAAYSAGKPAYGVGPGNAPCYVHQSANVAKAAQDIILGKSEDNGLLCSSPNSI
VADKAIDGALKAALTSSGGHELTTAEAQKLAAVLVTPQRLPNPVLVGRTAIQIAEIVGIAVPPGTR
ALIAVLEGVGRDHPLSIEKLCPVLSYFVVDDWREGCERCKQVLRYGGMGHTMSIHATDDDVILEFG
LHKPAFRIVVNTPTTHGSVGLTTGLDPAMTLGCGGHGGNITSDNISPLHLLNIKRVAYELRPAVAP
AQRSVGQQASMTDSGSSLRPVVPERVRTLDAPTLSARIDTFLASRGIAKDAALVVGRDGSPSRPSP
LVPSHPGDGPLGERAALPIPVVEVENAGPPVDFVCEDDVRQALRAGRRIRLAARAIVTPAARELGD
SNDLFVGE
SEQ ID NO: 53
Lysinibacillus sphaericus LsFAR (FAR13)
MATIDKDLLAIQEMRDAVSQANAAQAAYMQYSQQQVDSIVKAVADAAFKEAERLGKMAVEETGMGI
QNHKKMKNEVASRDVYEDIKDLKTVGVIGTDRLKKVTEIASPFGVIAGIVPTTNPTSTAIFKTLIS
LKTRNGLVLSPHPYAVKCTKEALDICRVAAENAGAPEGLLQCLTMSSMEATQQLMKHPQIHLILAT
GGGALVKAAYSSGKPAYGVGPGNVPAYIEKSANIQKAARQLVQSKSFDNGTICATEQAIIVDKAIA
EQVLAELKKQHAYILNAEEKLKMEKLISPVPGKVNPQIVGKSAVSLAHSIGIMVPEETKVLVGLET
MIGKNIPFSLEKLSPIFAFYTVEDSTEAKQVMVDLLNIGGRGHSCSIHTENAALAEQFSLDLPVSR
IVVNTLSSIGAVGGTTGLAPSFTLGCGTFGGNITSDNITARHLLNIKRMAYGIKDVEVPAPEFDLH
PVVENIKAQAPSLDTEVVQQIVDQVLKQITLQTK
SEQ ID NO: 54
Brevibacillus laterosporus BlFAR (FAR14)
MTLSDKDLLSVQEVRNLIKRSNTAQKALSKMSQEQIDTIVKAVANAGVVHANRLAKLAVEETGFGI
WEDKVIKNLFGSQVVYEHIKDLKTIGILNEDTVKKVIEVGVPVGVIAGLIPSTNPTSTVLYKSLIS
IKAGNSIIFSPHPNAKKCILETIKIIREAITSASGPEDIVTCMEMPTIQGTNELMKHKDVQLILAT
GGSAMVKAAYSSGTPAIGVGSGNGPAYIDKSADLQNAVKRIFDSATEDNGTICASEQSVVVERCMK
EKVIAEFKKQGAYFLNPEEKEKVGRFIMRENGSMNPQIVGKSVQHIANLAKITVPAGARLLIAEET
EVGYKVAFSREKLAPILGFYTADSMEKACDLCVEILLHEGAGHTCIIHAQDEEVIRYFGVRQPVSR
FLVNTPAALGGIGGTTNLVPALTLGCGAVGNNSTSDNVGPMNLINIRRIAYGVRELEALRPHQASC
NTQGGFGVNDDLVNSIIQKILLELRQSNCIS
SEQ ID NO: 55
Nocardiodes sp. NoFAR (FAR15)
MTHDELDSDLRSIQEARRLATAARAAQREFAHASQAEVDRICAAMADAVYREAARLGQLATDETGY
GVPAHKRLKVEFASRTVWESIRDVPTVGVLRRDEAKGIVEIGWPVGVIVGLCPSTNPNSTAIYKVL
ISVKARNACIIAPHPSAKAATYEAVRIMIEAGERAGMPKGLVGCMQEVSLPGSQELMRHYATSMIL
ATGGTPMVRAAHSMGKPALGVGPGNVPAYVDRSADVLAAATAIVNSKSFDCSTICATEQAVVADAP
IAGALRAEMERLGAYFVSAEEKAALERTVENPGGAMNPKAVGKSPQALAALAGIQVPEHARILVAE
LGSVGPQEPLSAEKLTTVLGWYVEDGWRAGCERSIELLKFGGDGHSLVIHATDEEVIMAFGLEKPA
FRILVNTWGTLGAIGATTGVMPALTLAPGGIGGAVVSDNITVTHLLNVKRLAFKLHEPPAAAYEHA
PDVRGAPRHDGPRSAEATPAARVAEPAAVSGDQVERIVRRVLSELGAGR
SEQ ID NO: 56
Shimwellia blattae SbFAR (FAR16)
MIELDNDLQSRQNARELVRNAKKAQAILATFSQQQIDAIVKNVAQEAAHHAEALAKMAAEETGEGN
WQDKVLKNRFASLRVYDAIKDIKTVGIIHDDPVKKVMDVGVPLGVICALVPSTNPTSTVIYKALIA
LKAGNAIIFSPHPGARQCSLKAIEIVKRAAQAAGAPAGSVDGITQLTLDATSELMHSKDVSLILAT
GGEGMVRAAYASGTPTISGGPGNGPAFIERSADIHQAVKDIVTSKTEDNGVICASEQSVIVERCIY
DEVHRELEAQGVYFMNDDEAAKMAALLLRANGTINPQVVGKTALHLSQMAGESVPASTRVLIARQS
TVSPKNPYSREKLCPVLGLYVEEDWKAACHRVVELLTNEGLGHTLVIHTRNQDVIRQFCLEKPVNR
ILINTPAALGGIGATTNISPALTLGCGAVGGGSSSDNVGPLNLLNIRKVGYGVRSVDELRAPGSRP
EPQPAMAAPAHCPQPSILDDARFTAPISAATGSDDRFATAGAATEATNDINEQNVERVIRQVLERL
GK
SEQ ID NO: 57
Oleisolibacter albus OaFAR (FAR17)
MNDGQIAAAVAKVLEAYGAAADAPPPPSAEPAAVTRPLATGQSAAGLIEQAIAKALAGGQPAPHVP
DPAGCGWKAGAGKTAADDPIARIIAKVVGEQSLTPAAPAATVAAPTDCGDGVFATMDEAVEASALA
QRQYLLCTMADRERFVQGIRDVVLCPATLETLARMAVEETGIGNVEHKLIKNRLAAEKTPGTEDLT
TDAQSGDNGLTLVEYSPYGVIGAITPTTNPTETIICNSIGMLAAGNSVVESPHPRARKVSLLLVQL
INRKLADLGAPANLVVTVSKPSIENTNALMAHPKVRMLVATGGPAIVKAVMSTGKKAIGAGAGNPP
VVVDETADIEKAAKDIVNGCSFDNNVPCVAEKEIIAVDQIADYLLENLKKNGAYELTDPAQIAALQ
KVVLTDKGGPQTSCVGKSAVWLLDKIGIAADASIKIIVMDVPKEHPFVQEELMMPILPLVRVKDVD
EAIDVAVEVEHGNRHTAIMHSTNVRKLTKMAKLIQTTIFVKNGPSYAGLGAGGEGYSTFTIAGPTG
EGLTSAKSFARRRKCVMVEALNVR
SEQ ID NO: 58
Lonepinella koalarum LkFAR (FAR18)
MNDVEISQAVSAVLSKYGVQPEQEKSTTNSVVTATVKETFPANLINQVVESCLAEKNVSASQLVTE
SKQNHVQSVKGIFSSMNDAVEAAWVAQRQYSLCSMKDRANFIAGIREVFQTETILAEISKLAVEET
GMGCYEHKLIKNRVAATKSPGIEDLATQALSGDQGLTLIELSAYGVIGSITPTTNPTETIICNSIG
MLAAGNSVVFSPHPRSRKVSLLAVKYINEKLAELGAPDNLVVTVDQPSIENTNALMVHPKVRMLVA
TGGPSIVRSVMSSGKKAIGAGAGNPPVVVDETADIEKAAKDIVNGCSEDNNLPCIAEKELIVVNTV
ADCLVRHMQNNGAYLLSEQTKIQLLQNVVLNDKKSGPNTGCVGKSAVYLLEKIGISAPNGTKIILV
ETDKKHSFVQHELMMPILPLIRVDNVDEAIDLAVEVEHGNRHTAIMHSTNVEKLTKMAKLIQTTIF
VKNGPSYAGIGVGGEGFATFTIAGPTGEGLTSARSFARQRRCTMVESLNIR
SEQ ID NO: 59
Breoghania corrubedonensis BcFAR (FAR19)
MNDQQISDAVTQVLDGYGAAPGAAPEQAVNQTTSATQGAPVAQASEFVSDMVANILADAGSVPAAK
GTPAFVPGSKRCCWQPAASGEDPMDAILAKALASGLGEKKAGTSAPAAKAAVTNPVVSDEEATTLG
DGVFATMDEAIAAAEQAQRQYLFCSMAARKAFVDGIREIFVDPATLERISTQTVEQTGMGNAAHKI
IKNRLAAEKSPGVEDLTTDAASGDGGLTLVEYSPFGVIGAITPTTNPTETIICNSIGMLAAGNAAV
FSPHPRATKVSLLTVKLINRKLAALGAPANLVVTVQKPSIENTNAMMAHPKVRMLVATGGPGIVKT
VMSTGKKAIGAGAGNPPVVVDETADIEKAAQDIVNGCSFDNNLPCIAEKEVIAVDQIADYLILCMQ
KCGAYLVTDAAVIDKLQALVINEKGGPQTACVGKSAVHLLDKVGIKVGDEVKVILIELPKEHPFVQ
EELMMPILPLVRSPSVDDAIDLAVDLEHGNRHTAMMHSTNVRKLTKMAKLIQTTIFVKNGPSYAGI
GVGGEGHATFTIAGPTGEGLTSARSFARKRRCVMVEALNVR
SEQ ID NO: 60
Kosakonia radicincitans KrFAR (FAR20)
MNTTELENMIRTILADNLTGIATAPGNIQHTIFARVEDAITASYDAYKKYLAEPLALRTRIITALK
EELAPWIKEMSERAAEETGMGNALDKISKNTAALNNTPGIEDLTTSALTGDGGMVLFELSPEGVIG
AIAPSTNPTETIINNTISMLAAGNAVYFSPHPGAKKVSLWLIEKIEDIIYRVSGIRNLVTTVAEPT
FDATREMMSDPRIALLVVTGGPAIVNMAMKTGKKVIGAGPGNPPVLVDETACPVKAAKDIVDGASF
DHNVLCIAEKCVIVVDSIADRLVENMQKNDAFLVKTPGDIARLRQVVINDKGEANKKLVGKSPAVI
LQAADLNTSTAPRLIIVEVEQDDPLVMVEQLMPVLPVVRVRDFETGLALALKVENDQHHTAIMHSQ
NVSRLNLAAKTMQTSIFVKNGPSYAGLGIEAEGFTTFTIATPTGEGTTSARSFARKRRCVLINGES
IR
SEQ ID NO: 61
Virgibacillus pantothenticus VpFAR (FAR21)
MPEFDKDLQSIQEARLLIASALKAQAELAEMSQKDIDQIVEHMAKAAFANAEKLAMLAHEETGEGN
VEDKIIKNKFASEKVYESIKNMKTVGILNDNEKQKVMEVAVPLGVIAGVIPSTNPTSTVIYKALIA
IKARNAIVESPHPGAKQCILETVKILNKAAIEAGAPAGAIGTLTILSMQGTSELMKHKDVALILAT
GGGAMVRAAYSSGNPAIGVGPGNGPAFIERTADIEKAVSRIISSKTEDNGVICASEQSVVVEQISE
SKVIQEFKRQGAYFLNEKESQLLSKFILRSNGTMNPKIVGKTAYSIAQMADITVPDNTTVLISRQT
EVSKVNPYAREKLCPILAFYTENDWISACDRCIELLNVEGIGHTLCIHSSDEAVIREFALRKPVSR
FLVNTPGALGGIGATTNLSPALTLGCGTVGGSSTSDNITPLNLLNIRRASYGIREKEDIRFTLNQD
ITFNGQNNIDLNSLNKEISKKEIAQLIEVALQKLAKEEA
SEQ ID NO: 62
Nakamurella multipartita NmFAR (FAR22)
MTAQVDGLDADLAGLVDVRAKARAARRAFDLLAGADQAQLDRYVRAMAEAGTRAAEELARLAVDET
GYGLYEHKIYKNRYNTGFVARWMLQRRAVGVLWVDEAARVTAVGAPMGVIAGIIPVTNPTSTALFK
CLAAVKSGNAIVLAPHPRAAGCTLRAAQIMAEAAVAAGAPDGLISCLEQPTLTVTAELMRRPEIAL
VLATGGPGMVRAAYSSGKPTIAVGAGNVPAYVGASVADPAEAAEMILTSKSEDNGTACVAEQSVVV
VDAVADAFLAAFAARGAHWLDAGQQEALARTLFDERGAMRPASVGQSAGTLARLSGFSVPRGTRVL
AARLDQVAADVPLSKEILGPVLSVYRVSDAAAGLDRCRQVLALGGEGHTAAVHAADPEEIAPFAAL
PAGRILINTPALFGGMGFSAEVDPSFQLGTGTWSGSICSDNVTPLHLINIKRIAHEVRPWRTVYDP
VEL
SEQ ID NO: 63
Aliishimia ponticola ApFAR (FAR23)
MDADLKSIAAARRCAETAFEAYRQFLGTDPAQIDAIVQAMAEAIEPEAARLGRMAVEETGYGNAQD
KRVKDLMNARGTAEWLRDVTTLGMLWRDDATKIAAFGEPMGVVAALIPVTNPSSTVIFKVLSAVKA
GNAIVCAPHPRGVKTGNEVVRIMARVAEQMGAPKGLIQCLDHVTVQGTAELMKHRRTSVVMATGGP
GMVRAAYSSGKPTLAVGAGNVPCYVHRSMAQELDEVAEMIIASKSFDWGTACVSEQAVIADPEIAR
ELRAELKLKGGYFCTAPEADRLAKVIFTGSQAMNPERVGQAPEVLAELAGESLPPRTKCLIAEETE
IGWHRPLSAEKLNPVLAFYEARDSAHGIALAHGIATFEGWGHSAVIHAHDPQVVADFARVPTGRVL
VNVPAIHGGAGYATDLEPSFMLGTGTWSGSITSDNVTALHLINIKRVAYGNRPWRDIYEEYGA
SEQ ID NO: 64
Serratia fonticola SEFAR (FAR24)
MNTTELESLIRTILTEQLAPETASNQRAIAIFDSVDEAISAAHHAFLRFQQSPLKTRSAIISALRE
QLKPHLPVLAERGASETGMGNSADKLLKNLAALENTPGVEDLATTAITGDGGMVLFEYSPFGVIGA
VAPSTNPTETIINNSISMLAAGNAVYFSPHPGAKNVSLDLIAMVEEIIFNSCGIRNLVVTVKEPSF
AATQEMMAHDKIALLAITGGPGIVAMGMKSGKKVIGAGAGNPPCLVDETAELVKAAQDIVAGASED
YNLPCIAEKALIVVDSVADRLLQQMQAFDALLIAQPQDVDRLRQVCLTEGHANKDLVGKSPAELLA
AAGLTCPAKLPRLLLVEVSGDDPLVTTEQLMPLLPVVRVKDEDTGLTLALQVEDGLHHTAVMHSQN
VSRLNLAARRLQTSIFVKNGPSYAGIGVGGEGFTTFTIATPTGEGTTSARTFARQRRCVLTNAFSI
R
SEQ ID NO: 65
Levilactobacillus brevis LbFAR (FAR25)
MNTENIEQAIRKILSEELSNPQSSTATNTTVPGKNGIFKTVNEAIAATKAAQENYADQPISVRNKV
IDAIREGFRPYIEDMAKRIHDETGMGTVSAKIAKLNNALYNTPGPEILQPEAETGDGGLVMYEYAP
FGVIGAVGPSTNPSETVIANAIMMLAGGNTLFFGAHPGAKNITRWTIEKLNELVADATGLHNLVVS
LETPSIESVQEVMQHPDVAMLSITGGPAVVHQALISGKKAVGAGAGNPPAMVDATANIALAAHNIV
DSAAFDNNILCTAEKEVVVEAAVKDELIMRMQQEGAFLVTDSADIEKLAQMTIGPKGAPDRKFVGK
DATYILDQAGISYTGTPTLIILEAAKDHPLVTTEMLMPILPVVCCPDEDSVLATATEVEGGLHHTA
SIHSENLPHINKAAHRLNTSIFVVNGPTYCGTGVATNGAHSGASALTIATPTGEGTATSKTYTRRR
RLNSPEGFSLRTWEA
SEQ ID NO: 66
Listeria innocua LiFAR (FAR26)
MESLELEQLVKKVLLEKLAEQKEVPTKTTTQGAKSGVEDTVDEAVQAAVIAQNCYKEKSLEERRNV
VKAIREALYPEIETIATRAVAETGMGNVTDKILKNTLAIEKTPGVEDLYTEVATGDNGMTLYELSP
YGVIGAVAPSTNPTETLICNSIGMLAAGNAVFYSPHPGAKNISLWLIEKLNTIVRESCGIDNLIVT
VAKPSIQAAQEMMNHPKVPLLVITGGPGVVLQAMQSGKKVIGAGAGNPPSIVDETANIEKAAADIV
DGASFDHNILCIAEKSVVAVDSIADELLFQMEKNGALHVTNPSDIQKLEKVAVTDKGVTNKKLVGK
SATEILKEAGIACDFTPRLIIVETEKSHPFATVELLMPIVPVVRVPDFDEALEVAIELEQGLHHTA
TMHSQNISRLNKAARDMQTSIFVKNGPSFAGLGFRGEGSTTFTIATPTGEGTTTARHFARRRRCVL
TDGESIR
SEQ ID NO: 67
Isosphaera pallida IpFAR (FAR27)
MQPSQTEELIRSVVQQVLSQMGTVFPGGLPGVGSGRLGVFPTVDAAAHAARDAFQRERGRPLADRR
RAIDVIRKVVIEGAEELGSMELAETKIGRRDHKIEKLVSAGQKIPGVEYLRTDNVSGDLGITLTDY
APFGVVGAVTPVTHSLPTLAGNAINILAAGNTVVFHPHPSGVGVALEGVRRENQGIREAIGLENLI
TIVESPTLESAQQLEDHREVDLLLVTGGPAVARAALSSRKRAIVAGPGNPPVVVDATACLDNAARS
IVTGAAYDNNLLCIGEKQVFAPSNVLDRLMDRMARYGGHRLDARQIEALTAAAFKTGEGGQPTLNK
ELVGQDPAVLAAHAGLSIPSGVQLLFGETPADHPFVQLEQMMPFVPFVRTADLDQAIEQAHRSEHG
YGHTAVLHSRDTATMTRMGRLMNCTIFVINGPCTAGLGGGGEGVLSYSIAGPTGEGVTTPLTFCRQ
QRTAVVGAMREL
SEQ ID NO: 68
Cytobacillus solani CsFAR (FAR28)
MNPAELPHQVHESGANGVEDRIEDAIEAGYIAQLNYVKQFQLKDREKIITAIREAVIENKEKLAQM
VFEETKLGRYEDKIAKHELVARKTPGTEDITTAAFSGDEGLTIIEQAPFGLVGAVTPVTNPTETII
NNSISLLAAGNAVVLNVHPSSKVSCAFVVNLINQAIKDTGGPENLVSMVKDPTLETLNRIIESPKV
KLLVGTGGPGMVKTLLKSGKKAIGAGAGNPPVIVDETADLKQAAKSIIEGASFDNNLLCIAEKELE
VIDSVADDLIFHMLNEGAYMLDQQQLSKLMSFALEENVHQEAGGCSLDNKREYHVSKDWVGKDAVS
FLRQLGIAHEEDIKLLICEVDEDHPFVQLEQMMPVFPIVRVGNLDEAIEMALLAEHGNRHTAIMHS
KNVDHLTKFARAIETTIFVKNASSLAGVGFGGEGHTTMTIAGPTGEGITSAKTFTRQRRCVLAEGG
FRIIG
SEQ ID NO: 69
Dongia mobilis DmFAR (FAR29)
MTAIVEAILAGDQRATGEADMMLESAEWAAAAFARFDRARIARIARAAADVAYAHAGELADAAVAE
TGFGVAAHKKIKNELTSKALHDLYAAEDFCSARIRAELKMVELPRPAGIVFALTPSTNPVCSVYYK
VLMALFTRNAIILSPHPMAKKCSVRAAKLMAEAAEAAGAPLGVIQVIEQPSLPLIEHVMKSPRVGV
ILATGGTPMVRAAYSSGNPAIGVGPGNAPVLVDDTADLNAAAKRIVESKSEDNSILCTNESVVIAV
ESIADRLLQSLKAAGAHVAKPEEVAALRELLFGRGTFNIDVLGKSAIEIGAKAGLKLPANTKIILA
EIDRIGIDEPLSKEKLCPVLGFLRVPHAQAGITQARALVRLSGAGHSAAIHSKHAPTILAYGAAVK
ALRVVVNAPCSQGAAGFGTHLAPAFTIGTGYFGSSSVGENVGPQHLVNWTRIAYNADAAEAFGGED
GLDAWAGGPALALGREAPMTLGRLQEGAEAVQNPPPQPPGRAGDTDEMALMREEIRRMVLEELRGA
LRS
SEQ ID NO: 70
Egibacter rhizosphaerae ErFAR (FAR30)
MSGLQPDEIQAIIERVRRRVGDDGEPDGARLRGEQALEASPGVSAGGGVHAGVQDALSAAGRAFAA
FDGAGLEARKRIISSVRQAMLEHGDRLAEMAQAETGLGRADDKTRKNRLVTTRTPGPEDLELDAET
GDRGMNVTEFAPFGIVAAITPTTNPTSTVINNTIAIVSAGNAVVENVHPNAKQVSAENVRLINEAI
MRAGGPPDLVTTIAEPTIESAQEVMNHPDVRLLLVTGGPGVVREALKTDKRAVTAGPGNPPAVVDE
TADIEQAAADIVAGGSEDNNIICTDEKTTIAVDSIADPLVRAMERSGAYVLAEHELRRLERVIFRE
LGEPNKPGRINPQWVGQDVQAILAEIGVRVGPEVRMAVARVPNEHSLVWTEQMMPVMPVTSVRDVD
QAIDLAVRSEHRFGHTASMHSSDVGRITRMGRAMNCSIFVANGPCYAGLGEGGEGFCSFSIATPTG
DGLTRPQTFSRERRMTIVGSLRMV
SEQ ID NO: 71
Pseudogracilibacillus auburnensis PaFAR (FAR31)
MQISEQVIQQLVEEVMNKLENNANQTAVTQLGQGVYPTVDEAVQAAKQANEALKKLSLATRVQMIE
NMRKTSIEHAEELASLAVQETGLGRVEDKRAKNILAAEKTPGMEDLPSTSYSGDNGLTIVEQAPLG
VIGSITPTTNPAATMINNSLSMVAAGNVVVYNPHPSAKQVTLQTMKLLNEAIVEAGGPKNVLTSVI
EPTLETSQQLMNHPEINALVVTGGGAVVKAAMSTGKKVIAAGPGNPPVVVDETANIKKAAKDIVEG
ASFDNNILCTAEKEVFVVQQVAQELKTEMLKNNAIELKAFQFEKLLKEILIEKDGKHYANKDYVGK
DATVLLQAAGIQADKDTKLIIVEVSNDHPLVHTEMLMPILPIVKVSSVDEAIKLAKTAEKGNRHTA
MMHSESITNLTKMAQTIEATIFVKNGPSVAGLGYESEGFTTLTIAGPTGEGLTSARTFTRQRRSVL
VDGLRII
SEQ ID NO: 72
Nostoc punctiforme NpFAR (FAR32)
MKPVLISLLEPLNKLSASEQMVLEKIMQPVSFSAGTYIFKEGSFADSCYILEEGIVRIETASESSS
NFVVNYLEAGTIFGEISLLDKMPRSATAYAQTNIIAKKILIQELEILLETSPRILICLWEMERKAA
PLGVRSLNELTIKKNHFVINPEVEVMLDKALLAQKEIQTWSEERIDALLLVIASSIAQHAESLATA
TVKATRVGDILDKVTKNKIASLGIYRSLVGKSGCGFISNNKINNVCEIASPMGIIFGMIPMTNPVA
TAVEKALICIKSRNALILSFPLSTKNVGTLVCEIIQEALIRQDAPVELIQWLKHRSSREQTEILMK
HKNVSLILATGGASMVKAAYSSGTPAIGVGPANTPTLICADANIQHAARTIIVSKSEDNGLICGSE
HNLIVDARVRKNFIKVLEQEGAAILTHEEKSYFSTVAFEDKSNRLQAKIIGQSAAKIAVMANIKRD
YHIKLIIVPNEFLSLDNPYAYEKMAPILSLFTVQNEIEGIDLCRSILQIEGKGHTAIIHTKNKAVV
RRFGLEMPASRILVNSPGVHGIIGLTSALDPSFTLGCGTFGGNSTTDNVTYSNLLNLKRVAYYQAP
KFLDPEIFKNTYPQWLLQLLNLLNFLKLNALVTFISQRVSIQLRR
Alcohol dehydrogenases (EC 1.1.1.1)
SEQ ID NO: 47
Saccharomyces cerevisiae scADH1 (ADH1)
MSIPETQKGVIFYESHGKLEYKDIPVPKPKANELLINVKYSGVCHTDLHAWHGDWPLPVKLPLVGG
HEGAGVVVGMGENVKGWKIGDYAGIKWLNGSCMACEYCELGNESNCPHADLSGYTHDGSFQQYATA
DAVQAAHIPQGTDLAQVAPILCAGITVYKALKSANLMAGHWVAISGAAGGLGSLAVQYAKAMGYRV
LGIDGGEGKEELFRSIGGEVFIDFTKEKDIVGAVLKATDGGAHGVINVSVSEAAIEASTRYVRANG
TTVLVGMPAGAKCCSDVENQVVKSISIVGSYVGNRADTREALDEFARGLVKSPIKVVGLSTLPEIY
EKMEKGQIVGRYVVDTSK
SEQ ID NO: 48
Marinobacter hydrocarbonoclasticus mhFAR (ADH2)
MAIQQVHHADTSSSKVLGQLRGKRVLITGTTGELGKVVLERLIRAVPDIGAIYLLIRGNKRHPDAR
SRFLEEIATSSVFDRLREADSEGFDAFLEERIHCVTGEVTEAGFGIGQEDYRKLATELDAVINSAA
SVNFREELDKALAINTLCLRNIAGMVDLNPKLAVLQVSTCYVNGMNSGQVTESVIKPAGEAVPRSP
DGFYEIEELVRLLQDKIEDVQARYSGKVLERKLVDLGIREANRYGWSDTYTFTKWLGEQLLMKALN
GRTLTILRPSIIESALEEPAPGWIEGVKVADAIILAYAREKVTLFPGKRSGIIDVIPVDLVANSII
LSLAEALGEPGRRRIYQCCSGGGNPISLGEFIDHLMAESKANYAAYDHLFYRQPSKPELAVNRALF
DLVISGVRLPLSLTDRVLKLLGNSRDLKMLRNLDTTQSLATIFGFYTAPDYIFRNDELMALANRMG
EVDKGLFPVDARLIDWELYLRKIHLAGLNRYALKERKVYSLKTARQRKKAA
SEQ ID NO: 49
Arabidopsis thaliana atCHR (ADH3)
MGKVLEKEAFGLAAKDESGILSPESESRRATGEKDVRFKVLFCGICHTDLSMAKNEWGLTTYPLVP
GHEIVGVVTEVGAKVKKFNAGDKVGVGYMAGSCRSCDSCNDGDENYCPKMILTSGAKNEDDTMTHG
GYSDHMVCAEDFIIRIPDNLPLDGAAPLLCAGVTVYSPMKYHGLDKPGMHIGVVGLGGLGHVAVKF
AKAMGTKVTVISTSERKRDEAVTRLGADAFLVSRDPKQMKDAMGTMDGIIDTVSATHPLLPLLGLL
KNKGKLVMVGAPAEPLELPVFPLIFGRKMVVGSMVGGIKETQEMVDLAGKHNITADIELISADYVN
TAMERLAKADVKYRFVIDVANTMKPTP
SEQ ID NO: 50
Solanum lycopersicum slADH2 (ADH4)
MSTTVGQVIRCKAAVAWEAGKPLVMEEVDVAPPQKMEVRLKILYTSLCHTDVYFWEAKGQNPVEPR
ILGHEAAGIVESVGEGVTDLAPGDHVLPVETGECKDCAHCKSEESNMCSLLRINTDRGVMLNDGKS
RFSINGNPIYHFVGTSTFSEYTVVHVGCVAKINPLAPLDKVCVLSCGISTGLGASLNVAKPTKGSS
VAIFGLGAVGLAAAEGARIAGASRIIGVDLNASRFEQAKKFGVTEFVNPKDYSKPVQEVIAEMTDG
GVDRSVECTGHIDAMISAFECVHDGWGVAVLVGVPHKEAVEKTHPLNELNERTLKGTFFGNYKPRS
DIPCVVEKYMNKELELEKFITHTLPFAEINKAFDLMLKGEGLRCIITMAD
SEQ ID NO: 51
Clostridium acetobutylicum caAdhE2 (ADH5)
MKVTNQKELKQKLNELREAQKKFATYTQEQVDKIFKQCAIAAAKERINLAKLAVEETGIGLVEDKI
IKNHFAAEYIYNKYKNEKTCGIIDHDDSLGITKVAEPIGIVAAIVPTTNPTSTAIFKSLISLKTRN
AIFFSPHPRAKKSTIAAAKLILDAAVKAGAPKNIIGWIDEPSIELSQDLMSEADIILATGGPSMVK
AAYSSGKPAIGVGAGNTPAIIDESADIDMAVSSIILSKTYDNGVICASEQSILVMNSIYEKVKEEF
VKRGSYILNQNEIAKIKETMEKNGAINADIVGKSAYIIAKMAGIEVPQTTKILIGEVQSVEKSELF
SHEKLSPVLAMYKVKDEDEALKKAQRLIELGGSGHTSSLYIDSQNNKDKVKEFGLAMKTSRTFINM
PSSQGASGDLYNFAIAPSFTLGCGTWGGNSVSQNVEPKHLLNIKSVAERRENMLWEKVPQKIYFKY
GCLRFALKELKDMNKKRAFIVTDKDLFKLGYVNKITKVLDEIDIKYSIFTDIKSDPTIDSVKKGAK
EMLNFEPDTIISIGGGSPMDAAKVMHLLYEYPEAEIENLAINEMDIRKRICNFPKLGTKAISVAIP
TTAGTGSEATPFAVITNDETGMKYPLTSYELTPNMAIIDTELMLNMPRKLTAATGIDALVHAIEAY
VSVMATDYTDELALRAIKMIFKYLPRAYKNGTNDIEAREKMAHASNIAGMAFANAFLGVCHSMAHK
LGAMHHVPHGIACAVLIEEVIKYNATDCPTKQTAFPQYKSPNAKRKYAEIAEYLNLKGTSDTEKVT
ALIEAISKLKIDLSIPQNISAAGINKKDFYNTLDKMSELAFDDQCTTANPRYPLISELKDIYIKSE
SEQ ID NO: 83
Escherichia coli EcYqhD (ADH6)
MNNFNLHTPTRILFGKGAIAGLREQIPHDARVLITYGGGSVKKTGVLDQVLDALKGMDVLEFGGIE
PNPAYETLMNAVKLVREQKVTFLLAVGGGSVLDGTKFIAAAANYPENIDPWHILQTGGKEIKSAIP
MGCVLTLPATGSESNAGAVISRKTTGDKQAFHSAHVQPVFAVLDPVYTYTLPPRQVANGVVDAFVH
TVEQYVTKPVDAKIQDRFAEGILLTLIEDGPKALKEPENYDVRANVMWAATQALNGLIGAGVPQDW
ATHMLGHELTAMHGLDHAQTLAIVLPALWNEKRDTKRAKLLQYAERVWNITEGSDDERIDAAIAAT
RNFFEQLGVPTHLSDYGLDGSSIPALLKKLEEHGMTQLGENHDITLDVSRRIYEAAR
SEQ ID NO: 84
Escherichia coli EcAdhE (ADH7)
MAVTNVAELNALVERVKKAQREYASFTQEQVDKIFRAAALAAADARIPLAKMAVAESGMGIVEDKV
IKNHFASEYIYNAYKDEKTCGVLSEDDTFGTITIAEPIGIICGIVPTTNPTSTAIFKSLISLKTRN
AIIFSPHPRAKDATNKAADIVLQAAIAAGAPKDLIGWIDQPSVELSNALMHHPDINLILATGGPGM
VKAAYSSGKPAIGVGAGNTPVVIDETADIKRAVASVLMSKTEDNGVICASEQSVVVVDSVYDAVRE
RFATHGGYLLQGKELKAVQDVILKNGALNAAIVGQPAYKIAELAGESVPENTKILIGEVTVVDESE
PFAHEKLSPTLAMYRAKDFEDAVEKAEKLVAMGGIGHTSCLYTDQDNQPARVSYFGQKMKTARILI
NTPASQGGIGDLYNFKLAPSLTLGCGSWGGNSISENVGPKHLINKKTVAKRAENMLWHKLPKSIYF
RRGSLPIALDEVITDGHKRALIVTDRELENNGYADQITSVLKAAGVETEVFFEVEADPTLSIVRKG
AELANSFKPDVIIALGGGSPMDAAKIMWVMYEHPETHFEELALREMDIRKRIYKFPKMGVKAKMIA
VTTTSGTGSEVTPFAVVTDDATGQKYPLADYALTPDMAIVDANLVMDMPKSLCAFGGLDAVTHAME
AYVSVLASEFSDGQALQALKLLKEYLPASYHEGSKNPVARERVHSAATIAGIAFANAFLGVCHSMA
HKLGSQFHIPHGLANALLICNVIRYNANDNPTKQTAFSQYDRPQARRRYAEIADHLGLSAPGDRTA
AKIEKLLAWLETLKAELGIPKSIREAGVQEADFLANVDKLSEDAFDDQCTGANPRYPLISELKQIL
LDTYYGRDYVEGETAAKKEAAPAKAEKKAKKSA
SEQ ID NO: 85
Escherichia coli EcYiaY (ADH8)
MAASTFFIPSVNVIGADSLTDAMNMMADYGFTRTLIVTDNMLTKLGMAGDVQKALEERNIFSVIYD
GTQPNPTTENVAAGLKLLKENNCDSVISLGGGSPHDCAKGIALVAANGGDIRDYEGVDRSAKPQLP
MIAINTTAGTASEMTRFCIITDEARHIKMAIVDKHVTPLLSVNDSSLMIGMPKSLTAATGMDALTH
AIEAYVSIAATPITDACALKAVTMIAENLPLAVEDGSNAKAREAMAYAQFLAGMAENNASLGYVHA
MAHQLGGFYNLPHGVCNAVLLPHVQVENSKVAAARLRDCAAAMGVNVTGKNDAEGAEACINAIREL
AKKVDIPAGLRDLNVKEEDFAVLATNALKDACGFTNPIQATHEEIVAIYRAAM
SEQ ID NO: 86
Escherichia coli EcFuco (ADH9)
MANRMILNETAWFGRGAVGALTDEVKRRGYQKALIVTDKTLVQCGVVAKVTDKMDAAGLAWAIYDG
VVPNPTITVVKEGLGVFQNSGADYLIAIGGGSPQDTCKAIGIISNNPEFADVRSLEGLSPTNKPSV
PILAIPTTAGTAAEVTINYVITDEEKRRKFVCVDPHDIPQVAFIDADMMDGMPPALKAATGVDALT
HAIEGYITRGAWALTDALHIKAIEIIAGALRGSVAGDKDAGEEMALGQYVAGMGFSNVGLGLVHGM
AHPLGAFYNTPHGVANAILLPHVMRYNADETGEKYRDIARVMGVKVEGMSLEEARNAAVEAVFALN
RDVGIPPHLRDVGVRKEDIPALAQAALDDVCTGGNPREATLEDIVELYHTAW
SEQ ID NO: 87
Escherichia coli EcHcxA (ADH10)
MPHNPIRVVVGPANYFSHPGSFNHLHDFFTDEQLSRAVWIYGKRAIAAAQTKLPPAFGLPGAKHIL
FRGHCSESDVQQLAAESGDDRSVVIGVGGGALLDTAKALARRLGLPFVAVPTIAATCAAWTPLSVW
YNDAGQALHYEIFDDANEMVLVEPEIILNAPQQYLLAGIGDTLAKWYEAVVLAPQPETLPLTVRLG
INNAQAIRDVLLNSSEQALSDQQNQQLTQSFCDVVDAIIAGGGMVGGLGDRFTRVAAAHAVINGLT
VLPQTEKFLHGTKVAYGILVQSALLGQDDVLAQLTGAYQRFHLPTTLAELEVDINNQAEIDKVIAH
TLRPVESIHYLPVTLTPDTLRAAFKKVESFKA
SEQ ID NO: 88
Escherichia coli EcAroB (ADH11)
MERIVVTLGERSYPITIASGLENEPASFLPLKSGEQVMLVTNETLAPLYLDKVRGVLEQAGVNVDS
VILPDGEQYKSLAVLDTVFTALLQKPHGRDTTLVALGGGVVGDLTGFAAASYQRGVRFIQVPTTLL
SQVDSSVGGKTAVNHPLGKNMIGAFYQPASVVVDLDCLKTLPPRELASGLAEVIKYGIILDGAFEN
WLEENLDALLRLDGPAMAYCIRRCCELKAEVVAADERETGLRALLNLGHTFGHAIEAEMGYGNWLH
GEAVAAGMVMAARTSERLGQESSAETQRIITLLKRAGLPVNGPREMSAQAYLPHMLRDKKVLAGEM
RLILPLAIGKSEVRSGVSHELVLNAIADCQSA
Acyl-ACP thioesterases (TES) (EC 3.1.2.14)
SEQ ID NO: 78
Lactobacillus plantarum LpTES (TES1)
MATLGANASLYSEQHRITYYECDRTGRATLTTLIDIAVLASEDQSDALGLTTEMVQSHGVGWVVTQ
YAIDITRMPRQDEVVTIAVRGSAYNPYFAYREFWIRDADGQQLAYITSIWVMMSQTTRRIVKILPE
LVAPYQSEVVKRIPRLPRPISFEATDTTITKPYHVRFFDIDPNRHVNNAHYFDWLVDTLPATELLQ
HDLVHVDVRYENEVKYGQTVTAHANILPSEVADQVTTSHLIEVDDEKCCEVTIQWRTLPEPIQ
SEQ ID NO: 79
Arabidopsis thaliana AtALT4 (TES2)
MIRVTGTAAPAMSVVFPTSWRQPVMLPLRSAKTFKPHTFLDLKGGKEMSEFHEVELKVRDYELDQF
GVVNNAVYANYCQHGMHEFLESIGINCDEVARSGEALAISELTMNFLAPLRSGDKFVVKVNISRTS
AARIYFDHSILKLPNQEVILEAKATVVWLDNKHRPVRIPSSIRSKEVHFLRQNDTV
SEQ ID NO: 80
Lactobacillus brevis LbTES (TES3)
MAANEFSETHRVVYYEADDTGQLTLAMLINLFVLVSEDQNDALGLSTAFVQSHGVGWVVTQYHLHI
DELPRTGAQVTIKTRATAYNRYFAYREYWLLDDAGQVLAYGEGIWVTMSYATRKITTIPAEVMAPY
HSEEQTRLPRLPRPDHFDEAVNQTLKPYTVRYFDIDGNGHVNNAHYFDWMLDVLPATFLRAHHPTD
VKIRFENEVQYGHQVTSELSQAAALTTQHMIKVGDLTAVKATIQWDNR
SEQ ID NO: 81
Bryantella formatexigens BfTES (TES4)
MIYMAYQYRSRIRYSEIGEDKKLTLPGLVNYFQDCSTFQSEALGIGLDTLGARQRAWLLASWKIVI
DRLPRLGEEVVTETWPYGFKGFQGNRNERMLDQEGHTLAAAASVWIYLNVESGHPCRIDGDVLEAY
ELEEELPLGPFSRKIPVPEESTERDSFLVMRSHLDTNHHVNNGQYILMAEEYLPEGEKVKQIRVEY
RKAAVLHDTIVPFVCTEPQRCTVSLCGSDEKPFAVVEFS
SEQ ID NO: 82
Cuphea palustris CpFatBlderivative (TES5)
MFDRKSKRPSMLMDSFGLERVVQDGLVFRQSFSIRSYEICADRTASMETVMNHVQETSLNQCKSIG
LLDDGFGRSPEMCKRDLIWVVTRMKIMVNRYPTWGDTIEVSTWLSQSGKIGMGRDWLISDCNTGEI
LVRATSVYAMMNQKTRRFSKLPHEVRQEFAPHELDSPPAIEDNDGKLQKEDVKTGDSIRKGLTPGW
YDLDVNQHVSNVKYIGWILESMPTEVLETQELCSLTLEYRRECGRDSVLESVTSMDPSKVGDRFQY
RHLLRLEDGADIMKGRTEWRPKNAGTNGAISTGKT
Enoyl-ACP reductase (ENR) (EC 1.3.1. 104)
SEQ ID NO: 89
Bacillus subtilis BsFabL (ENR1)
MAEQNKCALVTGSSRGVGKAAAIRLAENGYNIVINYARSKKAALETAEEIEKLGVKVLVVKANVGQ
PAKIKEMFQQIDETFGRLDVFVNNAASGVLRPVMELEETHWDWTMNINAKALLFCAQEAAKLMEKN
GGGHIVSISSLGSIRYLENYTTVGVSKAALEALTRYLAVELSPKQIIVNAVSGGAIDTDALKHEPN
REDLLEDARQNTPAGRMVEIKDMVDTVEFLVSSKADMIRGQTIIVDGGRSLLV

What is claimed is:

1. A microbial host cell producing cis-3-hexenol from hexanoic acid, the microbial cell expressing a recombinant biosynthetic pathway converting hexanoic acid to cis-3-hexenol and comprising:
(a) an acyl-CoA synthetase (ACS) converting hexanoic acid to hexanoyl-CoA;
(b) a short chain acyl-CoA oxidase (AOX) converting the hexanoyl-CoA to trans-2-hexenoyl-CoA; and
(c) a fatty acyl-CoA reductase (FAR) converting the cis-3-hexenoyl-CoA to cis-3-hexenal; and
optionally, the recombinant biosynthetic pathway comprises one or both of
(d) an enoyl-CoA isomerase (ECI) converting the trans-2-hexenoyl-CoA to cis-3-hexenoyl-CoA; and/or
(e) an alcohol dehydrogenase (ADH) converting the cis-3-hexenal to cis-3-hexenol.

2. The microbial host cell of claim 1, wherein the ACS comprises an amino acid sequence that is at least 70% identical to an amino acid sequence selected from SEQ ID NOs: 1-7.

3. The microbial host cell of claim 2, wherein the ACS comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 1-7.

4. The microbial host cell of claim 2, wherein the ACS comprises an amino acid sequence having from 1 to 20, from 1 to 10, or from 1 to 5 amino acid modifications independently selected from substitutions, insertions, and deletions with respect to an amino acid sequence selected from SEQ ID NOs: 1-7.

5. The microbial host cell of claim 1 or claim 2, wherein the ACS comprises an amino acid sequence that is at least 70% identical to the amino acid sequence of SEQ ID NO: 2.

6. The microbial host cell of claim 5, wherein the ACS comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 2.

7. The microbial host cell of claim 5, wherein the ACS comprises an amino acid sequence having from 1 to 20, from 1 to 10, or from 1 to 5 amino acid modifications independently selected from substitutions, insertions, and deletions with respect to the amino acid sequence of SEQ ID NO: 2.

8. The microbial host cell of claim 1 or claim 2, wherein the ACS comprises an amino acid sequence that is at least 70% identical to the amino acid sequence of SEQ ID NO: 3.

9. The microbial host cell of claim 8, wherein the ACS comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 3.

10. The microbial host cell of claim 8, wherein the ACS comprises an amino acid sequence having from 1 to 20, from 1 to 10, or from 1 to 5 amino acid modifications independently selected from substitutions, insertions, and deletions with respect to the amino acid sequence of SEQ ID NO: 3.

11. The microbial host cell of claim 1 or claim 2, wherein the ACS comprises an amino acid sequence that is at least 70% identical to the amino acid sequence of SEQ ID NO: 6.

12. The microbial host cell of claim 11, wherein the ACS comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 6.

13. The microbial host cell of claim 11, wherein the ACS comprises an amino acid sequence having from 1 to 20, from 1 to 10, or from 1 to 5 amino acid modifications independently selected from substitutions, insertions, and deletions with respect to the amino acid sequence of SEQ ID NO: 6.

14. The microbial host cell of any one of claims 1 to 4, wherein the AOX comprises an amino acid sequence that is at least 70% identical to an amino acid sequence selected from SEQ ID NOs: 8-20.

15. The microbial host cell of claim 14, wherein the AOX comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 8-20.

16. The microbial host cell of claim 14, wherein the AOX comprises an amino acid sequence having from 1 to 20, from 1 to 10, or from 1 to 5 amino acid modifications independently selected from substitutions, insertions, and deletions with respect to an amino acid sequence selected from SEQ ID NOs: 8-20.

17. The microbial host cell of claim 14, wherein the AOX comprises an amino acid sequence that is at least 70% identical to the amino acid sequence of SEQ ID NO: 11.

18. The microbial host cell of claim 17, wherein the AOX comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 11.

19. The microbial host cell of claim 17, wherein the AOX comprises an amino acid sequence having from 1 to 20, from 1 to 10, or from 1 to 5 amino acid modifications independently selected from substitutions, insertions, and deletions with respect to the amino acid sequence of SEQ ID NO: 11.

20. The microbial host cell of claim 14, wherein the AOX comprises an amino acid sequence that is at least 70% identical to the amino acid sequence of SEQ ID NO: 12.

21. The microbial host cell of claim 20, wherein the AOX comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 12.

22. The microbial host cell of claim 20, wherein the AOX comprises an amino acid sequence having from 1 to 20, from 1 to 10, or from 1 to 5 amino acid modifications independently selected from substitutions, insertions, and deletions with respect to the amino acid sequence of SEQ ID NO: 12.

23. The microbial host cell of claim 14, wherein the AOX comprises an amino acid sequence that is at least 70% identical to the amino acid sequence of SEQ ID NO: 14.

24. The microbial host cell of claim 23, wherein the AOX comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 14.

25. The microbial host cell of claim 23, wherein the AOX comprises an amino acid sequence having from 1 to 20, from 1 to 10, or from 1 to 5 amino acid modifications independently selected from substitutions, insertions, and deletions with respect to the amino acid sequence of SEQ ID NO: 14.

26. The microbial host cell of any one of claims 1 to 25, wherein the recombinant biosynthetic pathway comprises an ECI.

27. The microbial host cell of claim 26, wherein the ECI comprises an amino acid sequence that is at least 70% identical to an amino acid sequence selected from SEQ ID NOs: 21-35 and 73-77.

28. The microbial host cell of claim 27, wherein the ECI comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 21-35 and 73-77.

29. The microbial host cell of claim 27, wherein the ECI comprises an amino acid sequence having from 1 to 20, from 1 to 10, or from 1 to 5 amino acid modifications independently selected from substitutions, insertions, and deletions with respect to an amino acid sequence selected from SEQ ID NOs: 21-35 and 73-77.

30. The microbial host cell of claim 27, wherein the ECI comprises an amino acid sequence that is at least 70% identical to the amino acid sequence of SEQ ID NO: 30.

31. The microbial host cell of claim 30, wherein the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 30.

32. The microbial host cell of claim 30, wherein the ECI comprises an amino acid sequence having from 1 to 20, from 1 to 10, or from 1 to 5 amino acid modifications independently selected from substitutions, insertions, and deletions with respect to the amino acid sequence of SEQ ID NO: 30.

33. The microbial host cell of claim 27, wherein the ECI comprises an amino acid sequence that is at least 70% identical to the amino acid sequence of SEQ ID NO: 34.

34. The microbial host cell of claim 33, wherein the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 34.

35. The microbial host cell of claim 33, wherein the ECI comprises an amino acid sequence having from 1 to 20, from 1 to 10, or from 1 to 5 amino acid modifications independently selected from substitutions, insertions, and deletions with respect to the amino acid sequence of SEQ ID NO: 34.

36. The microbial host cell of claim 27, wherein the ECI comprises an amino acid sequence that is at least 70% identical to the amino acid sequence of SEQ ID NO: 35.

37. The microbial host cell of claim 36, wherein the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 35.

38. The microbial host cell of claim 36, wherein the ECI comprises an amino acid sequence having from 1 to 20, from 1 to 10, or from 1 to 5 amino acid modifications independently selected from substitutions, insertions, and deletions with respect to the amino acid sequence of SEQ ID NO: 35.

39. The microbial host cell of claim 27, wherein the ECI comprises an amino acid sequence that is at least 70% identical to the amino acid sequence of SEQ ID NO: 21.

40. The microbial host cell of claim 39, wherein the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 21.

41. The microbial host cell of claim 39, wherein the ECI comprises an amino acid sequence having from 1 to 20, from 1 to 10, or from 1 to 5 amino acid modifications independently selected from substitutions, insertions, and deletions with respect to the amino acid sequence of SEQ ID NO: 21.

42. The microbial host cell of claim 27, wherein the ECI comprises an amino acid sequence that is at least 70% identical to the amino acid sequence of SEQ ID NO: 73.

43. The microbial host cell of claim 42, wherein the ECI comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 73.

44. The microbial host cell of claim 42, wherein the ECI comprises an amino acid sequence having from 1 to 20, from 1 to 10, or from 1 to 5 amino acid modifications independently selected from substitutions, insertions, and deletions with respect to the amino acid sequence of SEQ ID NO: 73.

45. The microbial host cell of any one of claims 1 to 44, wherein the FAR comprises an amino acid sequence that is at least 70% identical to an amino acid sequence selected from SEQ ID NOs: 36-46 and 52-72.

46. The microbial host cell of claim 45, wherein the FAR comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 36-46 and 52-72.

47. The microbial host cell of claim 45, wherein the FAR comprises an amino acid sequence having from 1 to 20, from 1 to 10, or from 1 to 5 amino acid modifications independently selected from substitutions, insertions, and deletions with respect to an amino acid sequence selected from SEQ ID NOs: 36-46 and 52-72.

48. The microbial host cell of claim 45, wherein the FAR comprises an amino acid sequence that is at least 70% identical to the amino acid sequence of SEQ ID NO: 36.

49. The microbial host cell of claim 48, wherein the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 36.

50. The microbial host cell of claim 48, wherein the FAR comprises an amino acid sequence having from 1 to 20, from 1 to 10, or from 1 to 5 amino acid modifications independently selected from substitutions, insertions, and deletions with respect to the amino acid sequence of SEQ ID NO: 36.

51. The microbial host cell of claim 45, wherein the FAR comprises an amino acid sequence that is at least 70% identical to the amino acid sequence of SEQ ID NO: 37.

52. The microbial host cell of claim 51, wherein the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 37.

53. The microbial host cell of claim 51, wherein the FAR comprises an amino acid sequence having from 1 to 20, from 1 to 10, or from 1 to 5 amino acid modifications independently selected from substitutions, insertions, and deletions with respect to the amino acid sequence of SEQ ID NO: 37.

54. The microbial host cell of claim 45, wherein the FAR comprises an amino acid sequence that is at least 70% identical to the amino acid sequence of SEQ ID NO: 38.

55. The microbial host cell of claim 54, wherein the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 38.

56. The microbial host cell of claim 54, wherein the FAR comprises an amino acid sequence having from 1 to 20, from 1 to 10, or from 1 to 5 amino acid modifications independently selected from substitutions, insertions, and deletions with respect to the amino acid sequence of SEQ ID NO: 38.

57. The microbial host cell of claim 45, wherein the FAR comprises an amino acid sequence that is at least 70% identical to the amino acid sequence of SEQ ID NO: 58.

58. The microbial host cell of claim 57, wherein the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 58.

59. The microbial host cell of claim 57, wherein the FAR comprises an amino acid sequence having from 1 to 20, from 1 to 10, or from 1 to 5 amino acid modifications independently selected from substitutions, insertions, and deletions with respect to the amino acid sequence of SEQ ID NO: 58.

60. The microbial host cell of claim 45, wherein the FAR comprises an amino acid sequence that is at least 70% identical to the amino acid sequence of SEQ ID NO: 59.

61. The microbial host cell of claim 60, wherein the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 59.

62. The microbial host cell of claim 60, wherein the FAR comprises an amino acid sequence having from 1 to 20, from 1 to 10, or from 1 to 5 amino acid modifications independently selected from substitutions, insertions, and deletions with respect to the amino acid sequence of SEQ ID NO: 59.

63. The microbial host cell of claim 45, wherein the FAR comprises an amino acid sequence that is at least 70% identical to the amino acid sequence of SEQ ID NO: 64.

64. The microbial host cell of claim 63, wherein the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 64.

65. The microbial host cell of claim 63, wherein the FAR comprises an amino acid sequence having from 1 to 20, from 1 to 10, or from 1 to 5 amino acid modifications independently selected from substitutions, insertions, and deletions with respect to the amino acid sequence of SEQ ID NO: 64.

66. The microbial host cell of claim 45, wherein the FAR comprises an amino acid sequence that is at least 70% identical to the amino acid sequence of SEQ ID NO: 65.

67. The microbial host cell of claim 66, wherein the FAR comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 65.

68. The microbial host cell of claim 66, wherein the FAR comprises an amino acid sequence having from 1 to 20, from 1 to 10, or from 1 to 5 amino acid modifications independently selected from substitutions, insertions, and deletions with respect to the amino acid sequence of SEQ ID NO: 65.

69. The microbial host cell of any one of claims 1 to 67, wherein the recombinant biosynthetic pathway comprises an ADH.

70. The microbial host cell of claim 69, wherein the ADH comprises an amino acid sequence that is at least 70% identical to an amino acid sequence selected from SEQ ID NOs: 47-51 and 83-88.

71. The microbial host cell of claim 70, wherein the ADH comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 47-51 and 83-88.

72. The microbial host cell of claim 70, wherein the ADH comprises an amino acid sequence having from 1 to 20, from 1 to 10, or from 1 to 5 amino acid modifications independently selected from substitutions, insertions, and deletions with respect to an amino acid sequence selected from SEQ ID NOs: 47-51 and 83-88.

73. The microbial host cell of claim 70, wherein the ADH comprises an amino acid sequence that is at least 70% identical to the amino acid sequence of SEQ ID NO: 47.

74. The microbial host cell of claim 73, wherein the ADH comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 47.

75. The microbial host cell of claim 73, wherein the ADH comprises an amino acid sequence having from 1 to 20, from 1 to 10, or from 1 to 5 amino acid modifications independently selected from substitutions, insertions, and deletions with respect to the amino acid sequence of SEQ ID NO: 47.

76. The microbial host cell of claim 70, wherein the ADH comprises an amino acid sequence that is at least 70% identical to the amino acid sequence of SEQ ID NO: 83.

77. The microbial host cell of claim 76, wherein the ADH comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 83.

78. The microbial host cell of claim 76, wherein the ADH comprises an amino acid sequence having from 1 to 20, from 1 to 10, or from 1 to 5 amino acid modifications independently selected from substitutions, insertions, and deletions with respect to the amino acid sequence of SEQ ID NO: 83.

79. The microbial host cell of claim 70, wherein the ADH comprises an amino acid sequence that is at least 70% identical to the amino acid sequence of SEQ ID NO: 84.

80. The microbial host cell of claim 79, wherein the ADH comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 84.

81. The microbial host cell of claim 79, wherein the ADH comprises an amino acid sequence having from 1 to 20, from 1 to 10, or from 1 to 5 amino acid modifications independently selected from substitutions, insertions, and deletions with respect to the amino acid sequence of SEQ ID NO: 84.

82. The microbial host cell of claim 70, wherein the ADH comprises an amino acid sequence that is at least 70% identical to the amino acid sequence of SEQ ID NO: 87.

83. The microbial host cell of claim 82, wherein the ADH comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 87.

84. The microbial host cell of claim 82, wherein the ADH comprises an amino acid sequence having from 1 to 20, from 1 to 10, or from 1 to 5 amino acid modifications independently selected from substitutions, insertions, and deletions with respect to the amino acid sequence of SEQ ID NO: 87.

85. The microbial host cell of any one of claims 1-84, wherein the microbial strain expresses an acyl-acyl carrier protein (ACP) thioesterase (TES).

86. The microbial host cell of claim 85, wherein the TES comprises an amino acid sequence that is at least about 80%, or at least about 85% or at least about 90% or at least about 95%, at least about 96%, at least about 97%, at least about 98%, or at least about 99% identical to an amino acid sequence selected from SEQ ID NOs: 78-82.

87. The microbial host cell of claim 85, wherein the TES comprises an amino acid sequence having from 1 to 20, from 1 to 10, or from 1 to 5 amino acid modifications independently selected from substitutions, insertions, and deletions with respect to an amino acid sequence selected from SEQ ID NOs: 78-82.

88. The microbial host cell of claim 85, wherein the TES comprises an amino acid sequence that is at least 70% identical to the amino acid sequence of SEQ ID NO: 79.

89. The microbial host cell of claim 88, wherein the TES comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 79.

90. The microbial host cell of claim 88, wherein the TES comprises an amino acid sequence having from 1 to 20, from 1 to 10, or from 1 to 5 amino acid modifications independently selected from substitutions, insertions, and deletions with respect to the amino acid sequence of SEQ ID NO: 79.

91. The microbial host cell of claim 85, wherein the TES comprises an amino acid sequence that is at least 70% identical to the amino acid sequence of SEQ ID NO: 81.

92. The microbial host cell of claim 91, wherein the TES comprises an amino acid sequence that is at least about 80%, or at least about 85% or at least about 90% or at least about 95%, at least about 96%, at least about 97%, at least about 98%, or at least about 99% identical to the amino acid sequence of SEQ ID NO: 81.

93. The microbial host cell of claim 91, wherein the TES comprises an amino acid sequence having from 1 to 20, from 1 to 10, or from 1 to 5 amino acid modifications independently selected from substitutions, insertions, and deletions with respect to the amino acid sequence of SEQ ID NO: 81.

94. The microbial host cell of any one of claims 1-93, wherein the microbial strain expresses an enoyl-acyl-carrier-protein (ACP) reductase (ENR).

95. The microbial host cell of claim 94, wherein the ENR uses NADH and/or NADPH as a cofactor.

96. The microbial host cell of claim 94, wherein the ENR comprises an amino acid sequence that is at least about 80%, or at least about 85% or at least about 90% or at least about 95%, at least about 96%, at least about 97%, at least about 98%, or at least about 99% identical to an amino acid sequence of SEQ ID NO: 89.

97. The microbial host cell of claim 94, wherein the TES comprises an amino acid sequence having from 1 to 20, from 1 to 10, or from 1 to 5 amino acid modifications independently selected from substitutions, insertions, and deletions with respect to an amino acid sequence of SEQ ID NO: 89.

98. A microbial host cell producing cis-3-hexenal or a derivative thereof, the microbial cell expressing a recombinant biosynthetic pathway comprising: (a) an acyl-CoA synthetase (ACS) converting hexanoic acid to hexanoyl-CoA; (b) a short chain acyl-CoA oxidase (AOX) converting the hexanoyl-CoA to trans-2-hexenoyl-CoA; and (c) a fatty acyl-CoA reductase (FAR) converting the cis-3-hexenoyl-CoA to cis-3-hexenal.

99. The microbial host cell of claim 98, wherein the recombinant biosynthetic pathway comprises an enoyl-CoA isomerase (ECI) converting the trans-2-hexenoyl-CoA to cis-3-hexenoyl-CoA.

100. The microbial host cell of claim 98 or claim 99, wherein the recombinant biosynthetic pathway comprises an alcohol dehydrogenase (ADH) converting the cis-3-hexenal to cis-3-hexenol.

101. The microbial host cell of any one of claims 98-100, wherein the recombinant biosynthetic pathway comprises an enzyme that oxidizes cis-3-hexenoyl-CoA to cis-3-hexenoic acid.

102. The microbial host cell of any one of claims 98-100, wherein the recombinant biosynthetic pathway comprises an enzyme that oxidizes cis-3-hexenal to cis-3-hexenoic acid.

103. The microbial host cell of any one of claims 98-100, wherein the recombinant biosynthetic pathway comprises an enzyme that converts cis-3-hexenol to a cis-3-hexenoyl ester derivative.

104. The microbial host cell of any one of claims 98 to 103, wherein the cis-3-hexenoyl ester is selected from cis-3-hexenoyl acetate, cis-3-hexenoyl salicylate, cis-3-hexenoyl propionate, cis-3-hexenoyl formate, cis-3-hexenoyl butyrate, cis-3-hexenoyl hexanoate, cis-3-hexenoyl cis-3-hexenoate, cis-3-hexenoyl lactate, and cis-3-hexenoyl acetoacetate.

105. The microbial host cell of any one of claims 98-100, wherein the recombinant biosynthetic pathway comprises:
an enzyme that converts trans-3-hexenoyl-CoA to trans-3-hexenal; and/or
an enzyme that oxidizes trans-3-hexenoyl-CoA to trans-3-hexenoic acid.

106. The microbial host cell of any one of claims 98-100, wherein the recombinant biosynthetic pathway comprises an enzyme that oxidizes trans-3-hexenal to trans-3-hexenoic acid.

107. The microbial host cell of any one of claims 98-100, wherein the recombinant biosynthetic pathway comprises an enzyme that converts trans-3-hexenol to a trans-3-hexenoyl ester derivative.

108. The microbial host cell of any one of claim 98 to 100 or 104-106, wherein the trans-3-hexenoyl ester is selected from trans-3-hexenoyl acetate, trans-3-hexenoyl salicylate, trans-3-hexenoyl propionate, trans-3-hexenoyl formate, trans-3-hexenoyl butyrate, trans-3-hexenoyl hexanoate, trans-3-hexenoyl trans-3-hexenoate, trans-3-hexenoyl lactate, and trans-3-hexenoyl acetoacetate.

109. A microbial host cell producing trans-2-hexenal or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA.

110. A microbial host cell producing cis-2-hexenal or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA and (c) an enoyl-CoA isomerase (ECI) converting the trans-2-hexenoyl-CoA to cis-2-hexenoyl-CoA.

111. A microbial host cell producing cis-2-hexenol or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA; and (c) an enoyl-CoA isomerase (ECI) converting the trans-2-hexenoyl-CoA to cis-2-hexenoyl-CoA.

112. A microbial host cell producing trans-2-hexenol or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA.

113. A microbial host cell producing trans-3-hexenol or a derivative thereof, microbial host cell expressing a recombinant biosynthetic pathway comprising: (a) an acyl-CoA synthetase (ACS) converting hexanoic acid to hexanoyl-CoA; and (b) a short chain acyl-CoA oxidase (AOX) converting the hexanoyl-CoA to trans-2-hexenoyl-CoA; and (c) an enoyl-CoA isomerase (ECI) converting the trans-2-hexenoyl-CoA to trans-3-hexenoyl-CoA.

114. A microbial host cell producing cis-3-hexenol or a derivative thereof, microbial host cell expressing a recombinant biosynthetic pathway comprising: (a) an acyl-CoA synthetase (ACS) converting hexanoic acid to hexanoyl-CoA; (b) a short chain acyl-CoA oxidase (AOX) converting the hexanoyl-CoA to trans-2-hexenoyl-CoA; and (c) an enoyl-CoA isomerase (ECI) converting the trans-2-hexenoyl-CoA to cis-3-hexenoyl-CoA.

115. The microbial host cell of any one of claims 109 to 113, wherein the recombinant biosynthetic pathway comprises:
an enzyme that oxidizes converts trans-2-hexenoyl-CoA to trans-2-hexenal and/or converts cis-2-hexenoyl-CoA to cis-2-hexenal; and/or
an enzyme that converts trans-2-hexenoyl-CoA to trans-2-hexenal, and/or converts cis-2-hexenoyl-CoA to cis-2-hexenal.

116. The microbial host cell of claim 115, wherein the recombinant biosynthetic pathway expresses an enzyme that converts trans-2-hexenal to trans-2-hexenoic acid and/or cis-2-hexenal to sis-2-hexenoic acid.

117. The microbial host cell of claim 116, wherein the recombinant biosynthetic pathway expresses an enzyme that converts the trans-2-hexenol to a trans-2-hexenoyl ester and/or cis-2-hexenol to a cis-2-hexenoyl ester.

118. The microbial host cell of claim 117, wherein the trans-2-hexenoyl ester is selected from trans-2-hexenoyl propionate, trans-2-hexenoyl hexenoate, and ethyl trans-2-hexenoate.

119. The microbial host cell of claim 117, wherein the cis-2-hexenoyl ester is selected from cis-2-hexenoyl propionate, cis-2-hexenoyl hexenoate, and ethyl cis-2-hexenoate.

120. The microbial host cell of any one of claims 1 to 119, wherein the strain has increased expression or activity of one or more catalase enzymes.

121. The microbial host cell of claim 120, wherein the catalase is a cytosolic catalase.

122. The microbial host cell of claim 120, wherein the catalase is a peroxisomal catalase.

123. The microbial host cell of any one of claims 1 to 122, wherein the microbial host cell has one or more modifications that increase metabolic NADPH supply.

124. The microbial host cell of claim 123, wherein the modification(s) that increase metabolic NADPH supply:
(i) increase glycolytic flux through the oxidative pentose phosphate pathway;
(ii) express an alternative or exogenous NADPH biosynthesis route; and/or
(iii) increase production of NADPH via tricarboxylic acid intermediates.

125. The microbial host cell of claim 124, wherein the modifications result in increased glycolytic flux through the oxidative pentose phosphate pathway, and optionally comprise a deletion or reduced amount or activity of:
(A) glucose-6-phosphate isomerase; and/or
(B) phosphofructokinase.

126. The microbial host cell of claim 124 or claim 125, wherein the modifications result in increased glycolytic flux through the oxidative pentose phosphate pathway, and optionally comprise an increase in the amount or activity of:
(A) glucose-6-phosphate dehydrogenase; and/or
(B) 6-phosphogluconate dehydrogenase.

127. The microbial host cell of any one of claims 124 to 126, wherein the cell has an alternative or exogenous NADPH biosynthesis route, which optionally comprises bacterial transhydrogenase expression, and/or a NADP-dependent glyceraldehyde-3-phosphate dehydrogenase expression.

128. The microbial host cell of claim 127, wherein the cell:
expresses pntAB and/or gapN, or a variant thereof.

129. The microbial host cell of any one of claims 124 to 128, wherein the modifications result in increased production of NADPH via tricarboxylic acid intermediates, and the modifications optionally comprise increased expression or activity of a cytosolic NADP(+)-dependent isocitrate dehydrogenase.

130. The microbial host cell of any one of claims 1 to 129, wherein the microbial host cell has one or more modifications that downregulate β-oxidation and peroxisome metabolism.

131. The microbial host cell of claim 130, wherein the downregulation of β-oxidation and peroxisome metabolism is caused by a reduction in the amount or activity of one or more of:
(i) multifunctional β-oxidation enzyme;
(ii) peroxisomal membrane E3 ubiquitin ligase;
(iii) peroxisomal membrane protein;
(iv) one or more peroxisomal acyl-CoA oxidase;
(v) peroxisomal adenine nucleotide transporter;
(vi) one or more enoyl-CoA hydratase;
(vii) one or more 3-hydroxyacyl-CoA dehydratase;
(viii) enoyl-CoA hydratase/isomerase;
(ix) 3-hydroxyacyl-CoA dehydrogenase;
(x) 3-ketoacyl-CoA thiolase; and
(xi) acyl-CoA dehydrogenase.

132. The microbial host cell of any one of claims 1 to 131, wherein the microbial host cell has one or more modifications that reduce the amount or activity of:
citric acid cytoplasmic exporter; and/or
one or more NADPH dependent aldehyde reductases.

133. The microbial host cell of any one of claims 1 to 132, wherein the microbial host cell has one or more modifications that reduce neutral lipid biosynthesis.

134. The microbial host cell of claim 133, wherein the reduction of neutral lipid biosynthesis is caused by a reduction in the amount or activity of:
(i) diacylglycerol acyltransferase enzyme; and/or
(ii) acyl-CoA:sterol acyltransferase.

135. The microbial cell of any one of claims 1 to 134, wherein the microbial host cell has one or more modifications that reduce the amount or activity of one or more native aldehyde dehydrogenase, alcohol dehydrogenases and/or alcohol oxidase.

136. The microbial host cell of claim 135, wherein the aldehyde dehydrogenases is YALI0C03025g and/or YALI0F04444g, or a homolog thereof.

137. The microbial cell of any one of claims 1 to 136, wherein the microbial host cell has one or more modifications that reduce the amount or activity of one or more native multifunctional enzyme that is involved in the degradation of fatty acids via the β-oxidation cycle.

138. The microbial host cell of claim 137, wherein the multifunctional enzyme is encoded by fadB, or an ortholog, an analog, or homolog thereof.

139. The microbial cell of any one of claims 1 to 138, wherein the microbial host cell has one or more modifications that reduce the amount or activity of one or more native fatty acid oxidation complex 3-hydroxyacyl-CoA dehydrogenase/enoyl-CoA hydratase/3-hydroxybutyryl-CoA epimerase.

140. The microbial host cell of claim 139, wherein the fatty acid oxidation complex 3-hydroxyacyl-CoA dehydrogenase/enoyl-CoA hydratase/3-hydroxybutyryl-CoA epimerase is encoded by fadJ, or an ortholog, an analog, or homolog thereof.

141. The microbial cell of any one of claims 1 to 140, wherein the microbial host cell has one or more modifications that reduce the amount or activity of one or more native oxepin-CoA hydrolase/3-oxo-5,6-dehydrosuberyl-CoA semialdehyde dehydrogenase.

142. The microbial host cell of claim 141, wherein the oxepin-CoA hydrolase/3-oxo-5,6-dehydrosuberyl-CoA semialdehyde dehydrogenase is encoded by paaZ, or an ortholog, an analog, or homolog thereof.

143. The microbial host cell of any one of claims 130 to 142, wherein the one or more modifications comprises a complete or partial deletion or a null mutation or a hypomorphic mutation.

144. The microbial cell of any one of claims 1 to 143, wherein the microbial host cell has one or more modifications that increase the amount or activity of one or more native alcohol dehydrogenases and/or alcohol oxidase.

145. The microbial cell of any one of claims 1 to 144, wherein the microbial host cell is a yeast, optionally selected from Saccharomyces, Pichia, or Yarrowia.

146. The microbial cell of claim 145, wherein the microbial cell is Saccharomyces cerevisiae, Pichia pastoris, Yarrowia phangngensis and Yarrowia lipolytica.

147. The microbial cell of any one of claims 1 to 144, wherein the microbial host cell is a bacterium.

148. The microbial cell of claim 147, wherein the microbial cell is a bacterium selected from Escherichia spp., Bacillus spp., Corynebacterium spp., Rhodobacter spp., Zymomonas spp., Vibrio spp., and Pseudomonas spp.

149. The microbial cell of claim 148, wherein the microbial cell belongs to a bacterial host cell is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida.

150. The microbial cell of claim 149, wherein the microbial cell is Escherichia coli.

151. A method for making a compound selected from cis-3-hexenol, cis-3-hexenal, trans-3-hexenol, trans-3-hexenal or a derivative thereof, and trans-2-hexenal, cis-2-hexenal or a derivative thereof, the method comprising: culturing the microbial cell of any one of claims 1 to 150, and recovering said compound from the culture.

152. The method of claim 151, microbial host cell converts an hexanoic acid substrate to said compound.

153. The method of claim 152, wherein the hexanoic acid substrate is added to the culture.

154. The method of claim 153, wherein the hexanoic acid substrate is synthesized by the microbial cell.

155. The method of any one of claims 151 to 154, wherein the size of the culture is at least about 100 L, at least about 200 L, at least about 500 L, at least about 1,000 L, or at least about 10,000 L.

156. The method of any one of claims 151 to 155, wherein the culturing is conducted in a batch culture.

157. The method of any one of claims 151 to 156, wherein the culturing is conducted in a continuous culture, or a semi-continuous culture.

158. A method for making a product, comprising incorporating the compound selected from cis-3-hexenol, cis-3-hexenal, trans-3-hexenol, trans-3-hexenal or a derivative thereof, and trans-2-hexenal, cis-2-hexenal 1 or a derivative thereof made according to the method of any one of claims 133-142 into said product.

159. The method of claim 158, wherein the product is a flavour or fragrance product.

160. The method of any one of claims 151 to 159 wherein the compound is cis-3-hexenol.


SEQUENCE LISTING (89 sequences)

SEQ ID NO: 1 - protein, 566 aa, Escherichia coli
MHPTGPHLGPDVLFRESNMKVTLTFNEQRRAAYRQQGLWGDASLADYWQQTARAMPDKIAVVDNHGASYTYSALDHAASCLANWMLAKGIESGDRIAFQLPGWCEFTVIYLACLKIGAVSVPLLPSWREAELVWVLNKCQAKMFFAPTLFKQTRPVDLILPLQNQLPQLQQIVGVDKLAPATSSLSLSQIIADNTSLTTAITTHGDELAAVLFTSGTEGLPKGVMLTHNNILASERAYCARLNLTWQDVFMMPAPLGHATGFLHGVTAPFLIGARSVLLDIFTPDACLALLEQQRCTCMLGATPFVYDLLNVLEKQPADLSALRFFLCGGTTIPKKVARECQQRGIKLLSVYGSTESSPHAVVNLDDPLSRFMHTDGYAAAGVEIKVVDDARKTLPPGCEGEEASRGPNVFMGYFDEPELTARALDEEGWYYSGDLCRMDEAGYIKITGRKKDIIVRGGENISSREVEDILLQHPKIHDACVVAMSDERLGERSCAYVVLKAPHHSLSLEEVVAFFSRKRVAKYKYPEHIVVIEKLPRTTSGKIQKFLLRKDIMRRLTQDVCEEIE

SEQ ID NO: 2 - protein, 548 aa, Pseudomonas putida
MMVPTLEHELAPNEANHVPLSPLSFLKRAAQVYPQRDAVIYGARRYSYRQLHERSRALASALERVGVQPGERVAILAPNIPEMLEAHYGVPGAGAVLVCINIRLEGRSIAFILRHCAAKVLICDREFGAVANQALAMLDAPPLLVGIDDDQAERADLAHDLDYEAFLAQGDPARPLSAPQNEWQSIAINYTSGTTGDPKGVVLHHRGAYLNACAGALIFQLGPRSVYLWTLPMFHCNGWSHTWAVTLSGGTHVCLRKVQPDAINAAIAEHAVTHLSAAPVVMSMLIHAEHASAPPVPVSVITGGAAPPSAVIAAMEARGFNITHAYGMTESYGPSTLCLWQPGVDELPLEARAQFMSRQGVAHPLLEEATVLDTDTGRPVPADGLTLGELVVRGNTVMKGYLHNPEATRAALANGWLHTGDLAVLHLDGYVEIKDRAKDIIISGGENISSLEIEEVLYQHPEVVEAAVVARPDSRWGETPHAFVTLRADALASGDDLVRWCRERLAHFKAPRHVSLVDLPKTATGKIQKFVLREWARQQEAQIADAEH

SEQ ID NO: 3 - protein, 508 aa, Rhodopseudomonas palustris
MSFAYFDWIAHHAEVRPERIAVVDLASSRKISYRAMDERVDRLAAHLAALGVGRGDRVAVLALNAVETLEVQFACFRLGAIFVPLNVRLTVHELSYIVGDAAPRVLAHDDELAPMAKELKAACSVPHLLAFGAAYEAALAASPRLGASEPVTLDDVSTIMYTSGTTGKPKGAMITHGMTFINAVNLGIPAFISQRTVFLCVLPLFHTGGLNCYTNPVLHAGGTTLLMRAFDPGAALSIIGDPSVGLTHFFGVPSIYQFMCQHPAFAATDLSRLQIAGVGGAPMPVPLLKIWQERGCALVQGYGMTETSPAVLMLDADDAARKAGSAGKPVLHADLKIVGPDGDPVKPGEMGELWVKGPNITPGYWNRPDANRTSFTDGWLHTGDAARVDDEGFYYIVDRTKDMYISGGENVYPAEVEDVLYQLPEIAEAAVIGAPDPQWGETGVAVVALKPGQELSEAKLLAHCRERLARFKCPQRVSFVEALPRNATGKVHKPTLRERILVRETADA

SEQ ID NO: 4 - protein, 534 aa, Rhodopseudomonas palustris
MTSLEATGGVPGPGRIGRVAIGDILRKSARRFPDRVALTDGGRSVTYTELERDANRFANALVARGLKPGAKISTVCNNSIEFVKALFGIHRAGLVWVPINTMLGPDDMGYILDHAGVKVAVIDDNLHGQPERRAALEARGIDLIAINLAGKAADTGLPVFDQLIEGLSEIEPDVAFDDRDLAMIIYTSGTTSRPKGAMHCHLAVTMAVMSNAIEMQLSRKDGITGQFPLFHCAAHVLLLSYLIVGGQMAIMRGFDPVACMEAIQRNKLTVFIGLPLMYQVILDHPRRKEFDLSSLRCCIYTMAPMPRPLLERAIAELCPTFVQPSGQTEMYPATTMSQPDRQLARFGNYWGESTLVNETAIMDDAGNLLPPGEVGEIVHRGPNVMLGYYKDPEATEAARKFGWHHTGDLALIDEHGEVLFLDRKKDMIKSGGENVASIKIEETLLAHPSVMNAAVVGLPHPQWGEAVSGFVKLKPGASATEAEIVEHCKKHLGGFQVPKLLRIVDEMPMTATGKLRKVELRNQFTDHFMLGQTG

SEQ ID NO: 5 - protein, 721 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNMGALLEKRGKEFLGVKYKDPISSFSHFQEFSVRNPEVYWRTVLMDEMKISFSKDPECILRRDDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 6 - protein, 660 aa, Arabidopsis thaliana
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWIGGHSYVTYGPMLNGATVVVFEGAPNYPDPGRCWDIVDKYKVSIFYTAPTLVRSLMRDDDKFVTRHSRKSLRVLGSAGEPINPSAWRWFFNVVGDSRCPISDTWGQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADV

SEQ ID NO: 7 - protein, 744 aa, Saccharomyces cerevisiae
MAAPDYALTDLIESDPRFESLKTRLAGYTKGSDEYIEELYSQLPLTSYPRYKTFLKKQAVAISNPDNEAGFSSIYRSSLSSENLVSCVDKNLRTAYDHFMFSARRWPQRDCLGSRPIDKATGTWEETFRFESYSTVSKRCHNIGSGILSLVNTKRKRPLEANDFVVAILSHNNPEWILTDLACQAYSLTNTALYETLGPNTSEYILNLTEAPILIFAKSNMYHVLKMVPDMKFVNTLVCMDELTHDELRMLNESLLPVKCNSLNEKITFFSLEQVEQVGCFNKIPAIPPTPDSLYTISFTSGTTGLPKGVEMSHRNIASGIAFAFSTFRIPPDKRNQQLYDMCFLPLAHIFERMVIAYDLAIGFGIGFLHKPDPTVLVEDLKILKPYAVALVPRILTRFEAGIKNALDKSTVQRNVANTILDSKSARFTARGGPDKSIMNFLVYHRVLIDKIRDSLGLSNNSFIITGSAPISKDTLLFLRSALDIGIRQGYGLTETFAGVCLSEPFEKDVGSCGAIGISAECRLKSVPEMGYHADKDLKGELQIRGPQVFERYFKNPNETSKAVDQDGWFSTGDVAFIDGKGRISVIDRVKNFFKLAHGEYIAPEKIENIYLSSCPYITQIFVFGDPLKTFLVGIVGVDVDAAQPILAAKHPEVKTWTKEVLVENLNRNKKLRKEFLNKINKCTDGLQGFEKLHNIKVGLEPLTLEDDVVTPTFKIKRAKASKFFKDTLDQLYAEGSLVKTEKL

SEQ ID NO: 8 - protein, 404 aa, Geobacillus thermoleovorans
MENGDHAGTGDEDFGYIGKRGNETMNFRLSEEHEMLRKMVREFAENEVAPTAAERDEEERFDRGIFNKMAELGLTGIPWPEEYGGIGSDYLAYVIAVEELSRVCASTGVTLSAHISLASWPIYKFGNEEQKQKYLRALATGEKLGAYALSEPGAGSDVASMKTRAVKDGDHYILNGSKVWITNGGEAEIYVVFAVTDPEKRHKGISAFIVEKGTPGFSFGKKEKKLGIRSSPTTELIFEDCRIPKENLLGQEGEGFKIAMMTLDGGRNGIAAQAVGIAQGALDAAVDYAKQRVQFGKPIIEQQGVAFKLADMATAIEAARLLTYQAAWLESNGLPYGKASAMAKLFAGDTAMKVTVEAVQIFGGNGYTKDYPVERFMRDAKITQIYEGTQEIQRLVISRMLTRN

SEQ ID NO: 9 - protein, 411 aa, Geobacillus thermoleovorans
MKNKQLHRGMETMMNFDFTPEQEMLRQTVRKFVDKEIMPYIKEWDERGEFDRNIFKRLAELNLMGVCIPEEYGGMGMDYNSLAIVCEELERGDTAFRTAVSVHTGLNSLTLLQWGTEEQKQKYLVPQARGEKIGAFGLTEPNAGSDVASIQTTAVRDGDDYILNGQKTWISLADIADHFLVFAYTDKSKKHRGISAFIVERTMPGFSSRPIKGKLGIRSGNTGELFFDNVRVPKENLLGEEGEGFKIAMSALDNGRFTVAAGAVGLIMACLEASVKYCHERKTFGKEIGRHQLVQQMIARMEAGLQISRLLVYKVGFLKNEGRRCTRETSLAKWIACDYANQAADDAVQIHGAYGYSNEYPVERYLRNSKAPVIYEGTREIHTIMQAEYVLGYRQDKPLRKTLPAWRPAEN

SEQ ID NO: 10 - protein, 391 aa, Geobacillus thermoleovorans
MSATSEGGHFMEFRFTEEQEMMRQMVREFAAAEIAPFVERMEQGEFPRPILAKMAELGLMGITVPEQYGGAGMDFVSYIIAIHEISKVSPTVGVILSVHTSVGTNPILYFGTEEQKQKYVTKLARGEYLGAFCLTEPSAGSDAKSLKTKAVRRGDRYVLNGSKIFITNGGEADTYIVFARTNPEEAGSRGISAFIVEKGTPGMSIGKDEKKMGLHGSRTVTITFEDAEVPAENLLGQEGEGFKIAMANLDVGRIGIAAQALGIAEAAVEHAVAYAKERVQFGKPIIEQQGVAFKLADMATAAEAAKWLVYRAAWLRAQGLPCGKEASMAKLFASQTAMDNAIEAVQIFGGNGYTKDYPVERLFRDAKITQIYEGTSEIQRIVISKHLCQAC

SEQ ID NO: 11 - protein, 419 aa, Cucurbita pepo
  SITE 123: note = misc_feature - Xaa can be any naturally occurring amino acid
MTVDTKMNREDDKDKNARSAYFGSPALDVSVAFPQATAASVFPPSVSDYYQFNDLLTPEEQALRKKVRQCMEKEIAPIMTKYWEKAEFPFHVIPKLGNLCIAGGTIKVPAFCFLFKISYNFYXSSIIALCGSEEQKQKYLPSLAKLDTIACWALTEPENGSDASGLRTTATKVEGGWLIEGKKRWIGNSTFADLLVIFARNTITNEINGFIIKKNSPGLMVTKIENKIGLRIVQNGDIVMNKVFVPDEDRLPGVNSFKDTNKVLAVSRVMVAWQPIGIAMGVYDMCHRYLKEREQFGAPLAAFQVNQQKLVLMLGNVQAMFLMGWRLCKLYEKGTMTPGQASLGKAWITLRARETVALGRELLGGNGILSDFLVAKAFCDLEPIYTYEGTYDINTLVTGREITGVASFKPASLAKRSRL

SEQ ID NO: 12 - protein, 436 aa, Arabidopsis thaliana
MAVLSSADRASNEKKVKSSYFDLPPMEMSVAFPQATPASTFPPCTSDYYHFNDLLTPEEQAIRKKVRECMEKEVAPIMTEYWEKAEFPFHITPKLGAMGVAGGSIKGYGCPGLSITANAIATAEIARVDASCSTFILVHSSLGMLTIALCGSEAQKEKYLPSLAQLNTVACWALTEPDNGSDASGLGTTATKVEGGWKINGQKRWIGNSTFADLLIIFARNTTTNQINGFIVKKDAPGLKATKIPNKIGLRMVQNGDILLQNVFVPDEDRLPGVNSFQDTSKVLAVSRVMVAWQPIGISMGIYDMCHRYLKERKQFGAPLAAFQLNQQKLVQMLGNVQAMFLMGWRLCKLYETGQMTPGQASLGKAWISSKARETASLGRELLGGNGILADFLVAKAFCDLEPIYTYEGTYDINTLVTGREVTGIASFKPATRSRL

SEQ ID NO: 13 - protein, 709 aa, Candida tropicalis
MTFTKKNVSVSQGPDPRSSIQKERDSSKWNPQQMNYFLEGSVERSELMKALAQQMERDPILFTDGSYYDLTKDQQRELTAVKINRIARYREQESIDTFNKRLSLIGIFDPQVGTRIGVNLGLFLSCIRGNGTTSQLNYWANEKETADVKGIYGCFGMTELAHGSNVAGLETTATFDKESDEFVINTPHIGATKWWIGGAAHSATHCSVYARLIVDGQDYGVKTFVVPLRDSNHDLMPGVTVGDIGPKMGRDGIDNGWIQFSNVRIPRFFMLQKFCKVSAEGEVTLPPLEQLSYSALLGGRVMMVLDSYRMLARMSTIALRYAIGRRQFKGDNVDPNDPNALETQLIDYPLHQKRLFPYFVPPMSSPSVPSRLNTPSRPPWSNWTSPLKRTTPRLIFKSIDDMKSLFVDSGSLKSTATWLGAEAIDQCRQACGGHGHSSYNGFGKAYNDWVVQCTWEGDNNVLGMSVGKPIVKQVISIEDAGKTVRGSTAFLNQLKEYTGSNSSKVVLNTVADLDDIKTVIKAIEVAIIRLSQEAASIVKKESFDYVGAELVQLSKLKAHHYLLTEYIRRIDTFDQKELAPYLITLGKLYAATIVLDRFAGVFLTFNVASTEAITALASVQIPKLCAEVRPNVVAYTDSFQQSDMIVNSAIGRYDGDIYENYFDLVKLQNPPSKTKAPYSDALEAMLNRPTLDERERFQKSDETAAILSK

SEQ ID NO: 14 - protein, 700 aa, synthetic construct
MISPNLTANVEIDGKQYNTFTEPPKALAGERAKVKFPIKDMTEFLHGGEENVTMIERLMTELERDPVLNVSGDYDMPKEQLRETAVARIAALSGHWKKDTEKEALLRSQLHGIVDMGTRIRLGVHTGLFMGAIRGSGTKEQYDYWVRKGAADVKGFYGCFAMTELGHGSNVAGLETTATYIQDTDEFIINTPNTGATKWWIGGAAHSATHTACFARLLVDGKDYGVKIFVVQLRDVSSHSLMPGIALGDIGKKMGRDAIDNGWIQFTNVRIPRQNMLMKYAKVSSTGKVSQPPLAQLTYGALIGGRVTMIADSFFVSQRFITIALRYACVRRQFGTTPGQPETKIIDYPYHQRRLLPLLAFTYAMKMAADQSQIQYDQTTDLLQTIDPKDKGALGKAIVDLKELFASSAGLKAFTTWTCANIIDQCRQACGGHGYSGYNGFGQAYADWVVQCTWEGDNNVLCLSMGRGLIQSCLGHRKGKPLGSSVGYLANKGLEQATLSGRDLKDPKVLIEAWEKVANGAIQRATDKFVELTKGGLSPDQAFEELSQQRFQCAKIHTRKHLVTAFYERINASAKADVKPYLINLANLFTLWSIEEDSGLFLREGFLQPKDIDQVTELVNHYCKEVRDQVAGYTDAFGLSDWFINAPIGNYDGDVYKHYFAKVNQQNPAQNPRPPYYESTLRPFLFREDEDDDICELDEE

SEQ ID NO: 15 - protein, 383 aa, Thermobifida fusca
MDFTLTEEQTALRAMAAEFVDREITPHVVEWDRAESVPRDLVPRLGELGFLGMTIPEEYGGVGGDHVSYALVMEELGRGDSSVRGLVSVSLGLVAKSIATYGTEEQRQRWLPGLCSGELLGCFGLTEPDTGSDAASLSLKAVRDGDDYVLTGSKMFITNGTWADVAIIFARTGGPGPKGITAFLVPTDTPGLTRTTIHGKLGLRGQPTAQLFLDEVRVGPDAILGEENQGFRIAMAALDKGRVSVAAGAVGAARGALESALAHAKERVQFGRPIAGFQLVQEMLADMAVETDAARLLVWRAADLIDRGEPFSTAASMAKLYATETAVRAANNAMQVFGGYGYIDEYPPGKYLRDARVTTLYEGTSQIQKLIIGRALTGISAFA

SEQ ID NO: 16 - protein, 377 aa, Thermomonospora curvata
MDAQDFADVLAAVRSFVREQVIPREEEIEETDAIPESLRQAAREMGLFGYALPEEYGGLGLSLSEEVRLAFELGYASPAFRSMFGTSNGIAGQVLVNAGTEEQKRQWLPRLASGEVIGAFALTEAEAGSDPSTLSTTAVKDDDGNYVINGAKRFITNAPEADVFMVFARTGGPGSRGISTFLVERDAPGLKVGPPDEKMGQRGAHTAEVFFDNVRVPASAMVGPEGTGFRTAMASLAHGRLSIAAVCVGLAQRILDETVAYAKERAQGGRKIGEYQLVQGMIADSQAELYAGRAMVLEAARAYDAGEDRKLGPSCAKYFCSEMLGRVADRAVQVFGGMGYMRGVAVERFYRDARLFRIYEGTSQIQQLVIAKRLLAD

SEQ ID NO: 17 - protein, 380 aa, Geobacillus thermodenitrificans
MNFQLSEEHEMLRKMVREFAENEVAPTAAERDEEERFDRSIFNKMAELGLTGIPWPEEYGGIGSDYLAYVIAVEELSRVCASTGVTLSAHISLASWPIYKFGTEEQKQKYLRALATGEKLGAYGLSEPGAGSDVASMKTRAIKDGDHYVLNGSKVWITNGGEAEIYVVFAVTDPEKRHKGISAFIVEKGTPGFSIGKKEKKLGIRSSPTTELIFEDCRIPKENLLGQEGEGFKIAMMTLDGGRNGIAAQAVGIAQGALDAAVDYAKQRVQFGKPIAEQQGVSFKLADMATAIEAARLLTYQAAWLESNGLPYGKASAMAKLFAGDTAMKVTVDAVQIFGGNGYTKDYPVERFMRDAKITQIYEGTQEIQRLVISRMLTRD

SEQ ID NO: 18 - protein, 379 aa, Caldivirga maquilingensis
MHPLLEEVHELLRRSTVEFAEARVEPLAKAIEVNGEYPRQLMGELGKQGLLAPMAPPEYGGGGLDFRGEVIIIEEVAKHSPTLATLMEVQGALIIDSLLSHGSREVRERFLEGLVKGDLIASFALTEPCCGSDAGAIETRAVKDGGEWVINGRKAWVTSGQYADLYLVFARTGSIEERHRAITAFLVPRGGCIEVNPMGVMGIRGGGTAEVSLRDCRVPDEYRVGEVNRGFYLAMEKLNLGRTCVGAIGLGIAERAFTEAYDYAQVRRIYGGTLAELGVIQGYLAQMYTQVEALRGLIYLTAYMRDKGLSEFTKYAQAAKYLGSSTAVNVTRTAIQVMGAVGLSTESPLEYLYRDAKATEVYEGSNEVILYSLFKMLRK

SEQ ID NO: 19 - protein, 410 aa, Pyrobaculum aerophilum
MVFPFDTVEDYSIVLTREHEMFRKAVREFVEREIAPRAMEIEETDEVPRDILKKIAENGFFGIGIPEKYGGQGGDHRMAAILSEEFCRVLPGLSVYFGTNELFLTPIMLFGTEEQRQKYVPPIARGEKFGAFAVTEPCCGSDVAGIQTKAEKKGDKWVINGRKAFISSSDVADFFIVLARTYPPPDKKVRYLGLTFFIVEKDTPGFKVEQCYHKMGLYGNHACELVLENVEVPDENRVGEEGMGFLYAMETFDRTRIGVAAQAVGMAQAAFEKAFQYVHQRQAFGVPIAYFQAIQFSLVEMMAKIFTARLLTYLAAKLADEDRREFTFVASLAKFYATEVAEEVISEAINLHGGVGVIRETGVERFLRSVKITQIYEGANNIQKLVAYRQLIRLLKEKGQIPDEIARLVT

SEQ ID NO: 20 - protein, 384 aa, Thermoplasma acidophilum
MGNFGVTQDEELVLSFVRKFAEEELKPLAKEIDEKMEVPRKIIDRMKDLGLFATYIPKEYGGYGMSFPFLVRAIEEISKACPSTALVLDGALTLFAEPLIMFGSEDLKKRYLPRVAAGSVGGLAITEPGAGSDAAGISATAVKKGDRYVINGDKIFISNGRISDFFVLDAVTDPGKRHRGITAFVADRDTPGLKISRDIHKMGIRGSSTVELAFEDMEIPAENIVGKENEGFKVIMETLDAGRIGIAAQALGIAENALAEAIDYVKQRKQFGTEIANFEGIQFMIAEMATEIEAARYLTYVAAEKWQNKENTIEISAMAKMKASDVAMRVTTDALQLFGGYGYTTDLDAERHMRDAKITQIYEGTNQIQRLVIAKEILKKTRYY

SEQ ID NO: 21 - protein, 280 aa, Saccharomyces cerevisiae
MSQEIRQNEKISYRIEGPFFIIHLMNPDNLNALEGEDYIYLGELLELADRNRDVYFTIIQSSGRFFSSGADFKGIAKAQGDDTNKYPSETSKWVSNFVARNVYVTDAFIKHSKVLICCLNGPAIGLSAALVALCDIVYSINDKVYLLYPFANLGLITEGGTTVSLPLKFGTNTTYECLMFNKPFKYDIMCENGFISKNFNMPSSNAEAFNAKVLEELREKVKGLYLPSCLGMKKLLKSNHIDAFNKANSVEVNESLKYWVDGEPLKRFRQLGSKQRKHRL

SEQ ID NO: 22 - protein, 280 aa, synthetic construct
MVSIVKSSRIDSRKQGPFLIIFLNDEKTLNSLEGNDYLYLAHLLIENDKDADTSFTVIQSSGRFFSSGANFSSIVKENGKKNKDGELPKWAAAFLSRNTYVTTAFINHSKPLICCLNGPAVGLSAAIVMLCDIVYVMNNKVYLQFPFAKIGLVTEGAVAVTLPLKIGYARAQNVLFFNKRVTYDMLENTVSVKNYELDDWQQFNARVLEDLGKDVKNINMASIIGMKALIKETWKGHLLQANVSEVTDAMPFWIEGIPQSNFNKLLKKAESRKNKLKPKL

SEQ ID NO: 23 - protein, 263 aa, synthetic construct
MSPSYKIVNQVFVIVLDDPHTRNALTIPQFVQLAELLELADKHPHTTATLVVGRGPMFSAGANIKTVAELKNKTSSEILSEISAKNLYLVHLFTSHRKLLVVGLNGPVIGLTASLVALADLVYAQNQKVFMSFPFTNIGLTTECAAAVSLPHRLGLSTALEHVLLAKPLSAAKLHELGLVNRVFDLDDCDAFNDQLANMLTKELVGLDRDTIFVNKRLMRHTFDTQVRAQALQETMSGVADWTANKPQTAFDEIVAGKRKHKL

SEQ ID NO: 24 - protein, 276 aa, synthetic construct
MSDPAIKIEYRGRLAIVTINNEKKLNALNGNQYYALAQALREVATHDEVYITLLIGKGRYFSAGADVSLSQPSPEEQRIRSENPHRFWLQSFVANNLNITHAFYTHPKILVVGLNGPVIGLSAALVAFADFIYATPQTFLLTPFSSLGLVTEGGACRALIQRLGPARANEALIMSKRITAEELERAGFVNKIFSEVGKGEDEKFKQLVLNEINERLGEHLVGDSLLGIKKLIRRPETQILDAANVAEVFAGLDRFVSGVPQGEFEKIATGKKRHKL

SEQ ID NO: 25 - protein, 274 aa, synthetic construct
MEGEDILYEVRGKVTIITFNIPQKLNALNGEQYLLLAKLVERADKEEDTILTLIQSSGRFFSAGANFADKSLANTDAADLFSHEYWLNRFVARNTYLTELFHNHRKILAAAVNGPVIGLSASLLALCDLIYVKEEKDFYILTPFANLGLVAEGAASATLFLRLGWSKASEALLLAKPISGKDLNNLGFINKTYDGQFKTTEEFNQAVHDELVNAFENLHFDSIIQNKQLLKSNRDQLITSANSKEVIRGFNKWIEGVPQGRFVQLAQKDIKHKF

SEQ ID NO: 26 - protein, 234 aa, Thermomonospora curvata
MAVRIEDRGHVRIVTLDRPERRNAVDGPMADRIAEVFAAFERDGRARVAVLTGAGGTFCAGNDLKAIAAGEFPVPTRQAPPMRVTRAAPAKPVIAAIEGPCFGGGLELALWCDLRVASATAEFGLLNLVHGLPSMDAGTVRLPRLIGHARALEMIVTARRVPAAEAMGWGLLNRLVEPGRALDEALRLAEAVAALPQEPMLASRRSALQQWGRPEAEAFGREVELALAALGNPG

SEQ ID NO: 27 - protein, 269 aa, Thermomonospora curvata
MERLPISTAHCTVEQDGHVVIVTMNRPEARNALSTSMLVGMADAFAYISQTPEVRVGILTGAGGHFCSGADLKAMGTPPEDEREQRRAAEITNYHWKGLLREAVPTKPIIAAIEGYAVAGGTELLLGTDLRVVAEGATLGLYEAKRGLFPMGGSVVRLPRQIGYAPAMEILLTARSVTPQEALAMGLINRVVPDGQALSAARELADQIAACAPLSVQAILRAHRETFHLPEEEALKVSDEIGWPIFATEDAQEGPRAFREKRPPVFKGR

SEQ ID NO: 28 - protein, 256 aa, synthetic construct
MSGVRVERSGPVTTVILARPEVRNAIDGPTAAALAEAFRAFDADPDAAVAVLWGEGGTFCAGADLKAIGTERGNRVDPPPADAPLGCTRMRLSKPVIAAISGHAVAGGLELALWADLRVAEPDAVFGVFCRRWGVPLVDGGTVRLPRLIGMSRAMDMILTGRPVDAREAYDIGLVNRLVEPGRARAAAEELAADLARFPQTCLRGDRLSLLEQDGMTEDEALANEYRRGVNALGEAIEGAARFAAGAGRHGDFTDI

SEQ ID NO: 29 - protein, 272 aa, synthetic construct
MASDTDERVLLDRREGILVITLNRPERLNAVTADVSELMLDVLNGLMRDSRTHVVVLTGAGRGFCSGMDISGDDAIAAGASSEGRPQALYRGIALSGEVTARLREIPQPVIGALHGPVAGMGASWALACDLRVADPTTTLVLPFTNLGLSGGDCGLSWLLPRLVGPTKAADLLYRSQRLSFEQIRELGLVTEEADRGGDLDRALELADELLQRSPYGLRRTKELLNASLDAPGLRQQLLAETGVQTLAFFTQDLAEGMTAALERRPPRFANR

SEQ ID NO: 30 - protein, 240 aa, Arabidopsis thaliana
MCSLEKRDRLFILKLTGDGEHRLNPTLLDSLRSTINQIRSDPSFSQSVLITTSDGKFFSNGYDLALAESNPSLSVVMDAKLRSLVADLISLPMPTIAAVTGHASAAGCILAMSHDYVLMRRDRGFLYMSELDIELIVPAWFMAVIRGKIGSPAARRDVMLTAAKVTADVGVKMGIVDSAYGSAAETVEAAIKLGEEIVQRGGDGHVYGKMRESLLREVLIHTIGEYESGSSVVRSTGSKL

SEQ ID NO: 31 - protein, 238 aa, Arabidopsis thaliana
MCTLEKRGDLFLLTLTGEDEHRFHPDTIASVLSLLEQAKSQSTKGSVLITTGHGKFFSNGFDLAWAQSAGHGAIKRMHQMVKSFKPVLAALLDLPMPTIAALNGHAAASGLMFALSHDYVFMRKDRGVLYMSEVDIGLPVPDYFSALVVAKVGSGIARRELLLSGKKLKGEEAVALGIVDSAAHDSAEGVVEATVSLGESLAAKKWNGEVYATIRKSLYPELCRMVDLTANNLATHNL

SEQ ID NO: 32 - protein, 238 aa, Jatropha curcas
MCTLEKRDDIFILTFTGADEHRLSPTLIDSVLSALREVKSQATRGSVLITTSQGKFFSNGFDLAWARAAGSNSKAMERLRFMVQSFKPVVAEMISLPMPTVAALQGHAAAAGFLFSICHDYVLMRSDKGVLYMSEVDLGLPLPDYFASVFRDKLHSVSARRDVLLRGAKVKGEEAVRMGIVEAAYDSEAKLTDATIRLGEQLASRKWKGDVYKEIRKSLYPDICGVLALDEENIVSKL

SEQ ID NO: 33 - protein, 243 aa, synthetic construct
MCTLEKHGDVFVLTLTGSSNVDEHRLGPTAIDSILSAIAQAKAEATPGSALITTSHGKFFSNGFDLAWAQAAGSRTGARERLHYMFKSFRPLVAAMMDLPMPTVAAVNGHAAAAGFILALSHDYVVMRRDKGVLYMSEVDIGLTLPDYCAALFREKVGSATARRDVLLRAKKIKGEEALRMGLVEAAYDSEERVAEASMRLGKQLAGRKWAGEVYAEIRKSLYPDLCGVLGLDIKAVFSNSKL

SEQ ID NO: 34 - protein, 242 aa, Phoenix dactylifera
MCTLEKRGRVYLLTLTGDGEHRLNPTLLNAIRSALARVRSDSARAGGGSALVTAAEGKYFSNGFDLAWAKSSPANRTTIMTSAFQHVVADLMTLPMPTIAAVTGHASAAGFALALIHDYVVMRRDRGFLYMSELDIEIPIAEFVMALFRSKIADPRVRRDVLLRAPKITAAEAERKGIIERAVGGAAEAVETAVRMGEELAEKNWNGEVYASIRNAAFPEVCRALELAEEGDEETKRLASKL

SEQ ID NO: 35 - protein, 248 aa, Phoenix dactylifera
MCSLEKRGRVFVLTLTGDGEHRLGHDLIAAVRSALARVRAESAAGPGGYAFVTAAEGRFFSNGFDLAWANSAGSRSAARERLSSLVAVFRPIVADLMSLPMPTIAAVTGHAAAAGFMLAICHDYVAMRGDRGFLYMSELDIGLPFPPYFMALMRAKIADPRALRDVVLRAAKIPAADAKEKGIIDRVHGGAAETVEVAVRMGEELAARKWNGSIYASIRMAAFPELCRAVGLAEEGDEEKPKTVAAKL

SEQ ID NO: 36 - protein, 462 aa, synthetic construct
MTVNEQLVQDIIKNVVASMQLTQTNKTELGVFDDMNQAIEAAKEAQLVVKKMSMDQREKIISAIRKKTIEHAETLARMAVEETGMGNVGHKILKHQLVAEKTPGTEDITTTAWSGDRGLTLVEMGPFGVIGAITPCTNPSETIICNTIGMLAGGNTVVFNPHPAAIKTSNFAVQLINEASLSAGGPVNIACSVRKPTLDSSKIMMSHQDIPLIAATGGPGVVTAVLQSGKRGIGAGAGNPPVLVDETADIRKAAEDIINGCTFDNNLPCIAEKEVVAIDAIANELMNYMVKEQGCYAITKEQQEKLTNLVITPKGLNRNCVGKDARTLLGMIGIDVPSNIRCIIFEGEKEHPLISEELMMPILGIVRAKSFDDAVEKAVWLEHGNRHSAHIHSKNVDRITTYAKAIDTAILVKNAPSYAAIGFGGEGFCTFTIASRTGEGLTSASTFTKRRRCVMSDSLCIR

SEQ ID NO: 37 - protein, 467 aa, Thermoanaerobacterium saccharolyticum
MKVKEEDIEAIVKKVLSEFNFEKNTKSFRDFGVFQDMNDAIRAAKDAQKKLRNMSMESREKIIQNIRKKIMENKKILAEMGVSETGMGKVEHKIIKHELVALKTPGTEDIVTTAWSGDKGLTLVEMGPFGVIGTITPSTNPSETVLCNSIGMIAAGNSVVFNPHPGAVNVSNYAVKLVNEAVMEAGGPENLVASVEKPTLETGNIMFKSPDVSLLVATGGPGVVTSVLSSGKRAIGAGAGNPPVVVDETADIKKAAKDIVDGATFDNNLPCIAEKEVVSVDKITDELIYYMQQNGCYKIEGREIEKLIELVLDHKGGKITLNRKWVGKDAHLILKAIGIDADESVRCIIFEAEKDNPLVVEELMMPILGIVRAKNVDEAIMIATELEHGNRHSAHMHSKNVDNLTKFGKIIDTAIFVKNAPSYAALGYGGEGYCTFTIASRTGEGLTSARTFTKSRRCVLADGLSIR

SEQ ID NO: 38 - protein, 469 aa, Caldanaerobius polysaccharolyticus
MAGIREEDIELIVRRVLSNLDLKNLKAAVKKDIGVFEDMKQAISAAKKAQKELKSMSIEFREKIIQNIRKKTLENARIMAEMGVQETGMGKVEHKVLKHELVARKTPGTEDIITTAWSGDKGLTLVEMGPWGVIGAITPSTNPSETVICNSIGMIAAGNSVVFNPHPGAVGVSNYAVRLINEAVVEAGGPPNLAVSVAKPTLETAEIMFKHPDINLLVATGGPGVVTAVLSTGKRAIGAGAGNPPVVVDETADIRKAAKDIVDGATFDNNLPCIAEKEVIAVNKVADELIYYMKQNGCYMASKEEIEELKAMVLQTRDGKYYLNRKWVGKDASTLLKGIGVDVDDKVRCIIFEATKDHPFVVEELMMPILGIIRAENVDEAIAIAVELEHGFRHSAHMHSKNVDNLTKFARAIDTAIFVKNAPSYAAIGFGGEGYCTFTIASRTGEGLTSARTFTKSRRCVLADGLSIR

SEQ ID NO: 39 - protein, 468 aa, Clostridium beijerinckii
MNKDTLIPTTKDLKLKTNVENINLKNYKDNSSCFGVFENVENAINSAVHAQKILSLHYTKEQREKIITEIRKAALENKEVLATMILEETHMGRYEDKILKHELVAKYTPGTEDLTTTAWSGDNGLTVVEMSPYGVIGAITPSTNPTETVICNSIGMIAAGNAVVFNGHPGAKKCVAFAIEMINKAIISCGGPENLVTTIKNPTMESLDAIIKHPLIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYFINKKWVGKDAKLFSDEIDVESPSNIKCIVCEVNANHPFVMTELMMPILPIVRVKDIDEAVKYTKIAEQNRKHSAYIYSKNIDNLNRFEREIDTTIFVKNAKSFAGVGYEAEGFTTFTIAGSTGEGITSARNFTRQRRCVLAG

SEQ ID NO: 40 - protein, 491 aa, Clostridium kluyveri
MEIMDKDLQSIQEVRTLIAKAKKAQAEFKNFSQEAVNKVIEKIAKATEVEAVKLAKLAYEDTGYGKWEDKVIKNKFSSIVVYNYIKDLKTVGILKEDKEKKLIDIAVPLGVIAGLIPSTNPTSTAIFKVLIALKAGNAIVFSPHPTAVRSITETVKIMQKAAVEAGAPDGLIQCMSILTVEGTAELMKNKDTALILATGGEGMVRAAYSSGTPAIGVGPGNGPCFIERTADIPTAVRKVIGSDTFDNGVICASEQSIIAETVKKAEIIEEFKRQKGYFLNAEESEKVGKILLRANGTPNPAIVGKDVQALAKLAGISIPSDAVILLSEQTDVSPKNPYAKEKLAPVLAFYTVEDWHEACEKSLALLHNQGSGHTLIIHSQNEEIIREFALKKPVSRILVNSPGSLGGIGGATNLVPSLTLGCGAVGGSATSDNVGPENLFNIRKVAYGTTTVEEIREAFGVGAASSSAPAEPEDNEDVQAIVKAIMAKLNL

SEQ ID NO: 41 - protein, 316 aa, Escherichia coli
MSKRKVAIIGSGNIGTDLMIKILRHGQHLEMAVMVGIDPQSDGLARARRMGVATTHEGVIGLMNMPEFADIDIVFDATSAGAHVKNDAALREAKPDIRLIDLTPAAIGPYCVPVVNLEANVDQLNVNMVTCGGQATIPMVAAVSRVARVHYAEIIASIASKSAGPGTRANIDEFTETTSRAIEVVGGAAKGKAIIVLNPAEPPLMMRDTVYVLSDEASQDDIEASINEMAEAVQAYVPGYRLKQRVQFEVIPQDKPVNLPGVGQFSGLKTAVWLEVEGAAHYLPAYAGNLDIMTSSALATAEKMAQSLARKAGEAA

SEQ ID NO: 42 - protein, 661 aa, Marinobacter hydrocarbonoclasticus
MNYFLTGGTGFIGRFLVEKLLARGGTVYVLVREQSQDKLERLRERWGADDKQVKAVIGDLTSKNLGIDAKTLKSLKGNIDHVFHLAAVYDMGADEEAQAATNIEGTRAAVQAAEAMGAKHFHHVSSIAAAGLFKGIFREDMFEEAEKLDHPYLRTKHESEKVVREECKVPFRIYRPGMVIGHSETGEMDKVDGPYYFFKMIQKIRHALPQWVPTIGIEGGRLNIVPVDFVVDALDHIAHLEGEDGNCFHLVDSDPYKVGEILNIFCEAGHAPRMGMRIDSRMFGFIPPFIRQSIKNLPPVKRITGALLDDMGIPPSVMSFINYPTRFDTRELERVLKGTDIEVPRLPSYAPVIWDYWERNLDPDLFKDRTLKGTVEGKVCVVTGATSGIGLATAEKLAEAGAILVIGARTKETLDEVAASLEAKGGNVHAYQCDFSDMDDCDRFVKTVLDNHGHVDVLVNNAGRSIRRSLALSFDRFHDFERTMQLNYFGSVRLIMGFAPAMLERRRGHVVNISSIGVLTNAPRFSAYVSSKSALDAFSRCAAAEWSDRNVTFTTINMPLVKTPMIAPTKIYDSVPTLTPDEAAQMVADAIVYRPKRIATRLGVFAQVLHALAPKMGEIIMNTGYRMFPDSPAAAGSKSGEKPKVSTEQVAFAAIMRGIYW

SEQ ID NO: 43 - protein, 659 aa, synthetic construct
MGYFVTGATGFIGRHLVSRLFERKGTVYVLVRKESQKKLDDIARRMGWDTKRVMAVAGDMTRANCGLSAAQMRSLKGKVRHFFHLAAIYDLTADADAQYAANVEGTRHALDLAAALGAGCFHHASSIAAAGLYPGVFREDMFEEAEGLDDPYLRTKHESEGLVRKEKRLKWRIYRPGIVVGHSRTGEIDKIDGPYYFFTFIKKMRAMLPQWMPTLGIEGGRINLVPVDYVADAMDYIAHKPKLDGHTFHLVDPAPQRVGEVLNTFARAAHAPEMTMRLDARMFAFLPSAVRLAVGNLPPVKRMIAMLLRDFGIPKQALKFITYPTRFDNRETERALRGSGIAVPPLESYAWRLWDYWERHLDPDLFVDRSLKSRVKGKVVVITGGSSGIGLAAARKIAEAGAITVLCARGEDELFKVRDELRAAGGKVFAYTADLSDMADCDRFVQTVLKEHGAVDILVNNAGRSIRRSIELSFDRFHDFERTMQLNYFGSLRLIMGFLPGMIERRRGHIINISSIGVLANSPRFSAYVASKAALDAFSRCAQGELSGKGISFTTINMPLVKTPMIAPTKIYEAVPTLTPEEAADLVVRGIIEKPSRISTRLGTFAAVVNALAPKAYEVIMNTAFELFPDSAAAKGGGKEAQPSNEQIAFAALMRGVHW

SEQ ID NO: 44 - protein, 662 aa, Thermomonas hydrothermalis
MAYLVTGGTGFIGRFLIDNLLKRKGTIHVLVRKASLKKFEALAKQRGWDRKRVVPLVGDMAAPGCGVSAAQVRALAGKIRHVFHLAALYDLTASADQMYQANVEGTRHALDLAAALKAGCFHHVSSIAAAGLYPGVFREDMFEEAEGLDDPYLRTKHESEGLVRHETRIKWRIYRPGMVVGHSQTGEIDKIDGPYYFFTLIKKMRQMLPPWMPMLGLEGGRINIVPVDFVADALDHIAHKPRLDGHCFHLTDPDPLRVGEVLNVFARAAHAPEMSMRLDARMFAFVPPSVRMAVGSLPPVRRIVGALLRDFRIPRQVLKFITYPTRFDNREAERALKGSGIAVPRLEDYAWRLWDYWERHLDPDLFIDRSLKGTVAGKVVVITGGSSGIGLATARKVAAAGAITIIVARGQEELFKARDEMQAAGGKVFAYTADLSDMADCDRLVAEVLKAHGHVDILINNAGRSIRRSIELSYDRFHDFERTMQLNYFGSLRLIMGFLPTMVQRRKGHIINISSIGVLANSPRFSAYVASKAALDAFSRCAQGELSGKGIHFTTINMPLVKTPMIAPTKVYENVPTLTPDEAADLVVKGIIEKPSRIATRLGIFAAVINAVAPRAYEAVMNTAFELFPDSAAAKGDKQALRDETPSQEQIAFAALMRGVHW

SEQ ID NO: 45 - protein, 453 aa, Heliothis virescens
MVVLTSKETKPSVAEFYAGKSVFITGGTGFLGKVFIEKLLYSCPDIVNIYMLIREKKGLSVSERIKQFLDDPLFTRLKDKRPADLEKIVLIPGDITAPDLGITAANEKMLIEKVSVIIHSAATVKFNEPLPTAWKINVEGTRMMLALSRRMKRIEVFIHISTAYTNTNREVVDEILYPAPADIDQVYQYVKEGISEEDTEKILNGRPNTYTFTKALTEHLVAENQAYVPTIIVRPSVVAAIKDEPLKGWLGNWFGATGLTVFTAKGLNRVIYGHSNYIVDLIPVDYVANLVIAAGAKSNTSSELKVYNCCSSSCNPVKIGTLMSMFADDAIKQKSYAMPLPGWYIFTKYKWLVLLLTFLFQVIPAYITDLSRHLVGKSPRYIKLQSLVNQTRSSIDFFTNHSWVMKADRVRELYASLSPADKYLFPCDPVNINWTQYLQDYCWGVRNFLEKKT

SEQ ID NO: 46 - protein, 516 aa, Melipona quadrifasciata
MSTISSAQNTSVKDFYRDRCVFVTGVTGFMGKVLVEKLLRSCPGLKTIYVLIRNKKGQDAHQRLRALLNGPLFDKLRRDAPNELQKVIAVPGDITEHELGISESDQNILIRNVSVVFHSAATVKFDEALKISVTINMIGTKQLLNLCHRMQNLEALIHVSTAYCNCDRTDIAEQIYPLIAEPEEIFALTKVMDDKMIDDITPILIGKRPNTYTFTKALAERMLETESDYLPIAIVRPTIVLSSYKEPVAGWLDNWNGPTGIIAAAGKGFFRSMLCRDDMIADLVPVDIVINLMIVAAWKTATNRTKTIPIYNCCTGQQNPITWKKFVDLTFKYSRLHPYNDVIWYPGGNCHTSTIINKICMLLQHIVPAHILDFTHRLKGKTANMVTLQTKLEKATKYLEYFTMQQWSFRDDNVRELNEQLSPEDRQIFAFDVRQIDWSLYLEHYILGIRHFLLKENPDTLPAARVHLRKLYWFHKAVQFGVLLVLLRFLLLRSTVTQNACYSLVSLVLRMCRIIV

SEQ ID NO: 47 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 48 - protein, 513 aa, Marinobacter hydrocarbonoclasticus
MAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADAIILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAA

SEQ ID NO: 49 - protein, 357 aa, Arabidopsis thaliana
MGKVLEKEAFGLAAKDESGILSPFSFSRRATGEKDVRFKVLFCGICHTDLSMAKNEWGLTTYPLVPGHEIVGVVTEVGAKVKKFNAGDKVGVGYMAGSCRSCDSCNDGDENYCPKMILTSGAKNFDDTMTHGGYSDHMVCAEDFIIRIPDNLPLDGAAPLLCAGVTVYSPMKYHGLDKPGMHIGVVGLGGLGHVAVKFAKAMGTKVTVISTSERKRDEAVTRLGADAFLVSRDPKQMKDAMGTMDGIIDTVSATHPLLPLLGLLKNKGKLVMVGAPAEPLELPVFPLIFGRKMVVGSMVGGIKETQEMVDLAGKHNITADIELISADYVNTAMERLAKADVKYRFVIDVANTMKPTP

SEQ ID NO: 50 - protein, 380 aa, Solanum lycopersicum
MSTTVGQVIRCKAAVAWEAGKPLVMEEVDVAPPQKMEVRLKILYTSLCHTDVYFWEAKGQNPVFPRILGHEAAGIVESVGEGVTDLAPGDHVLPVFTGECKDCAHCKSEESNMCSLLRINTDRGVMLNDGKSRFSINGNPIYHFVGTSTFSEYTVVHVGCVAKINPLAPLDKVCVLSCGISTGLGASLNVAKPTKGSSVAIFGLGAVGLAAAEGARIAGASRIIGVDLNASRFEQAKKFGVTEFVNPKDYSKPVQEVIAEMTDGGVDRSVECTGHIDAMISAFECVHDGWGVAVLVGVPHKEAVFKTHPLNFLNERTLKGTFFGNYKPRSDIPCVVEKYMNKELELEKFITHTLPFAEINKAFDLMLKGEGLRCIITMAD

SEQ ID NO: 51 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 52 - protein, 602 aa, synthetic construct
MADVPAAKPHSDRDLASMAEARTLARAARVAQAQLAELPQDRIDAIVDAMAAAVRQEVDALARLACEETGFGVYADKIIKNRFAAEQVHEFIRPMRTVGVLRRDETRKIIEIAEPFGVVAAIVPSTNPTSTAIYKILIALKARCAIVISPHPSAARCISRTAEIMAAAAARAGAPADVIGWMSVVTLEGTQELMRQREVAVILATGGLGLVRAAYSAGKPAYGVGPGNAPCYVHQSANVAKAAQDIILGKSFDNGLLCSSPNSIVADKAIDGALKAALTSSGGHFLTTAEAQKLAAVLVTPQRLPNPVLVGRTAIQIAEIVGIAVPPGTRALIAVLEGVGRDHPLSIEKLCPVLSYFVVDDWREGCERCKQVLRYGGMGHTMSIHATDDDVILEFGLHKPAFRIVVNTPTTHGSVGLTTGLDPAMTLGCGGHGGNITSDNISPLHLLNIKRVAYELRPAVAPAQRSVGQQASMTDSGSSLRPVVPERVRTLDAPTLSARIDTFLASRGIAKDAALVVGRDGSPSRPSPLVPSHPGDGPLGERAALPIPVVEVENAGPPVDFVCEDDVRQALRAGRRIRLAARAIVTPAARELGDSNDLFVGE

SEQ ID NO: 53 - protein, 496 aa, Lysinibacillus sphaericus
MATIDKDLLAIQEMRDAVSQANAAQAAYMQYSQQQVDSIVKAVADAAFKEAERLGKMAVEETGMGIQNHKKMKNEVASRDVYEDIKDLKTVGVIGTDRLKKVTEIASPFGVIAGIVPTTNPTSTAIFKTLISLKTRNGLVLSPHPYAVKCTKEALDICRVAAENAGAPEGLLQCLTMSSMEATQQLMKHPQIHLILATGGGALVKAAYSSGKPAYGVGPGNVPAYIEKSANIQKAARQLVQSKSFDNGTICATEQAIIVDKAIAEQVLAELKKQHAYILNAEEKLKMEKLISPVPGKVNPQIVGKSAVSLAHSIGIMVPEETKVLVGLETMIGKNIPFSLEKLSPIFAFYTVEDSTEAKQVMVDLLNIGGRGHSCSIHTENAALAEQFSLDLPVSRIVVNTLSSIGAVGGTTGLAPSFTLGCGTFGGNITSDNITARHLLNIKRMAYGIKDVEVPAPEFDLHPVVENIKAQAPSLDTEVVQQIVDQVLKQITLQTK

SEQ ID NO: 54 - protein, 493 aa, Brevibacillus laterosporus
MTLSDKDLLSVQEVRNLIKRSNTAQKALSKMSQEQIDTIVKAVANAGVVHANRLAKLAVEETGFGIWEDKVIKNLFGSQVVYEHIKDLKTIGILNEDTVKKVIEVGVPVGVIAGLIPSTNPTSTVLYKSLISIKAGNSIIFSPHPNAKKCILETIKIIREAITSASGPEDIVTCMEMPTIQGTNELMKHKDVQLILATGGSAMVKAAYSSGTPAIGVGSGNGPAYIDKSADLQNAVKRIFDSATFDNGTICASEQSVVVERCMKEKVIAEFKKQGAYFLNPEEKEKVGRFIMRENGSMNPQIVGKSVQHIANLAKITVPAGARLLIAEETEVGYKVAFSREKLAPILGFYTADSMEKACDLCVEILLHEGAGHTCIIHAQDEEVIRYFGVRQPVSRFLVNTPAALGGIGGTTNLVPALTLGCGAVGNNSTSDNVGPMNLINIRRIAYGVRELEALRPHQASCNTQGGFGVNDDLVNSIIQKILLELRQSNCIS

SEQ ID NO: 55 - protein, 511 aa, synthetic construct
MTHDELDSDLRSIQEARRLATAARAAQREFAHASQAEVDRICAAMADAVYREAARLGQLATDETGYGVPAHKRLKVEFASRTVWESIRDVPTVGVLRRDEAKGIVEIGWPVGVIVGLCPSTNPNSTAIYKVLISVKARNACIIAPHPSAKAATYEAVRIMIEAGERAGMPKGLVGCMQEVSLPGSQELMRHYATSMILATGGTPMVRAAHSMGKPALGVGPGNVPAYVDRSADVLAAATAIVNSKSFDCSTICATEQAVVADAPIAGALRAEMERLGAYFVSAEEKAALERTVFNPGGAMNPKAVGKSPQALAALAGIQVPEHARILVAELGSVGPQEPLSAEKLTTVLGWYVEDGWRAGCERSIELLKFGGDGHSLVIHATDEEVIMAFGLEKPAFRILVNTWGTLGAIGATTGVMPALTLAPGGIGGAVVSDNITVTHLLNVKRLAFKLHEPPAAAYEHAPDVRGAPRHDGPRSAEATPAARVAEPAAVSGDQVERIVRRVLSELGAGR

SEQ ID NO: 56 - protein, 530 aa, Shimwellia blattae
MIELDNDLQSRQNARELVRNAKKAQAILATFSQQQIDAIVKNVAQEAAHHAEALAKMAAEETGFGNWQDKVLKNRFASLRVYDAIKDIKTVGIIHDDPVKKVMDVGVPLGVICALVPSTNPTSTVIYKALIALKAGNAIIFSPHPGARQCSLKAIEIVKRAAQAAGAPAGSVDGITQLTLDATSELMHSKDVSLILATGGEGMVRAAYASGTPTISGGPGNGPAFIERSADIHQAVKDIVTSKTFDNGVICASEQSVIVERCIYDEVHRELEAQGVYFMNDDEAAKMAALLLRANGTINPQVVGKTALHLSQMAGFSVPASTRVLIARQSTVSPKNPYSREKLCPVLGLYVEEDWKAACHRVVELLTNEGLGHTLVIHTRNQDVIRQFCLEKPVNRILINTPAALGGIGATTNISPALTLGCGAVGGGSSSDNVGPLNLLNIRKVGYGVRSVDELRAPGSRPEPQPAMAAPAHCPQPSILDDARFTAPISAATGSDDRFATAGAATEATNDINEQNVERVIRQVLERLGK

SEQ ID NO: 57 - protein, 552 aa, synthetic construct
MNDGQIAAAVAKVLEAYGAAADAPPPPSAEPAAVTRPLATGQSAAGLIEQAIAKALAGGQPAPHVPDPAGCGWKAGAGKTAADDPIARIIAKVVGEQSLTPAAPAATVAAPTDCGDGVFATMDEAVEASALAQRQYLLCTMADRERFVQGIRDVVLCPATLETLARMAVEETGIGNVEHKLIKNRLAAEKTPGTEDLTTDAQSGDNGLTLVEYSPYGVIGAITPTTNPTETIICNSIGMLAAGNSVVFSPHPRARKVSLLLVQLINRKLADLGAPANLVVTVSKPSIENTNALMAHPKVRMLVATGGPAIVKAVMSTGKKAIGAGAGNPPVVVDETADIEKAAKDIVNGCSFDNNVPCVAEKEIIAVDQIADYLLFNLKKNGAYELTDPAQIAALQKVVLTDKGGPQTSCVGKSAVWLLDKIGIAADASIKIIVMDVPKEHPFVQEELMMPILPLVRVKDVDEAIDVAVEVEHGNRHTAIMHSTNVRKLTKMAKLIQTTIFVKNGPSYAGLGAGGEGYSTFTIAGPTGEGLTSAKSFARRRKCVMVEALNVR

SEQ ID NO: 58 - protein, 513 aa, Lonepinella koalarum
MNDVEISQAVSAVLSKYGVQPEQEKSTTNSVVTATVKETFPANLINQVVESCLAEKNVSASQLVTESKQNHVQSVKGIFSSMNDAVEAAWVAQRQYSLCSMKDRANFIAGIREVFQTETILAEISKLAVEETGMGCYEHKLIKNRVAATKSPGIEDLATQALSGDQGLTLIELSAYGVIGSITPTTNPTETIICNSIGMLAAGNSVVFSPHPRSRKVSLLAVKYINEKLAELGAPDNLVVTVDQPSIENTNALMVHPKVRMLVATGGPSIVRSVMSSGKKAIGAGAGNPPVVVDETADIEKAAKDIVNGCSFDNNLPCIAEKELIVVNTVADCLVRHMQNNGAYLLSEQTKIQLLQNVVLNDKKSGPNTGCVGKSAVYLLEKIGISAPNGTKIILVETDKKHSFVQHELMMPILPLIRVDNVDEAIDLAVEVEHGNRHTAIMHSTNVEKLTKMAKLIQTTIFVKNGPSYAGIGVGGEGFATFTIAGPTGEGLTSARSFARQRRCTMVESLNIR

SEQ ID NO: 59 - protein, 569 aa, Breoghania corrubedonensis
MNDQQISDAVTQVLDGYGAAPGAAPEQAVNQTTSATQGAPVAQASEFVSDMVANILADAGSVPAAKGTPAFVPGSKRCCWQPAASGEDPMDAILAKALASGLGEKKAGTSAPAAKAAVTNPVVSDEEATTLGDGVFATMDEAIAAAEQAQRQYLFCSMAARKAFVDGIREIFVDPATLERISTQTVEQTGMGNAAHKIIKNRLAAEKSPGVEDLTTDAASGDGGLTLVEYSPFGVIGAITPTTNPTETIICNSIGMLAAGNAAVFSPHPRATKVSLLTVKLINRKLAALGAPANLVVTVQKPSIENTNAMMAHPKVRMLVATGGPGIVKTVMSTGKKAIGAGAGNPPVVVDETADIEKAAQDIVNGCSFDNNLPCIAEKEVIAVDQIADYLILCMQKCGAYLVTDAAVIDKLQALVINEKGGPQTACVGKSAVHLLDKVGIKVGDEVKVILIELPKEHPFVQEELMMPILPLVRSPSVDDAIDLAVDLEHGNRHTAMMHSTNVRKLTKMAKLIQTTIFVKNGPSYAGIGVGGEGHATFTIAGPTGEGLTSARSFARKRRCVMVEALNVR

SEQ ID NO: 60 - protein, 464 aa, synthetic construct
MNTTELENMIRTILADNLTGIATAPGNIQHTIFARVEDAITASYDAYKKYLAEPLALRTRIITALKEELAPWIKEMSERAAEETGMGNALDKISKNTAALNNTPGIEDLTTSALTGDGGMVLFELSPFGVIGAIAPSTNPTETIINNTISMLAAGNAVYFSPHPGAKKVSLWLIEKIEDIIYRVSGIRNLVTTVAEPTFDATREMMSDPRIALLVVTGGPAIVNMAMKTGKKVIGAGPGNPPVLVDETACPVKAAKDIVDGASFDHNVLCIAEKCVIVVDSIADRLVENMQKNDAFLVKTPGDIARLRQVVINDKGEANKKLVGKSPAVILQAADLNTSTAPRLIIVEVEQDDPLVMVEQLMPVLPVVRVRDFETGLALALKVENDQHHTAIMHSQNVSRLNLAAKTMQTSIFVKNGPSYAGLGIEAEGFTTFTIATPTGEGTTSARSFARKRRCVLTNGFSIR

SEQ ID NO: 61 - protein, 501 aa, Virgibacillus pantothenticus
MPEFDKDLQSIQEARLLIASALKAQAELAEMSQKDIDQIVEHMAKAAFANAEKLAMLAHEETGFGNVEDKIIKNKFASEKVYESIKNMKTVGILNDNEKQKVMEVAVPLGVIAGVIPSTNPTSTVIYKALIAIKARNAIVFSPHPGAKQCILETVKILNKAAIEAGAPAGAIGTLTILSMQGTSELMKHKDVALILATGGGAMVRAAYSSGNPAIGVGPGNGPAFIERTADIEKAVSRIISSKTFDNGVICASEQSVVVEQISESKVIQEFKRQGAYFLNEKESQLLSKFILRSNGTMNPKIVGKTAYSIAQMADITVPDNTTVLISRQTEVSKVNPYAREKLCPILAFYTENDWISACDRCIELLNVEGIGHTLCIHSSDEAVIREFALRKPVSRFLVNTPGALGGIGATTNLSPALTLGCGTVGGSSTSDNITPLNLLNIRRASYGIREKEDIRFTLNQDITFNGQNNIDLNSLNKEISKKEIAQLIEVALQKLAKEEA

SEQ ID NO: 62 - protein, 465 aa, Nakamurella multipartita
MTAQVDGLDADLAGLVDVRAKARAARRAFDLLAGADQAQLDRYVRAMAEAGTRAAEELARLAVDETGYGLYEHKIYKNRYNTGFVARWMLQRRAVGVLWVDEAARVTAVGAPMGVIAGIIPVTNPTSTALFKCLAAVKSGNAIVLAPHPRAAGCTLRAAQIMAEAAVAAGAPDGLISCLEQPTLTVTAELMRRPEIALVLATGGPGMVRAAYSSGKPTIAVGAGNVPAYVGASVADPAEAAEMILTSKSFDNGTACVAEQSVVVVDAVADAFLAAFAARGAHWLDAGQQEALARTLFDERGAMRPASVGQSAGTLARLSGFSVPRGTRVLAARLDQVAADVPLSKEILGPVLSVYRVSDAAAGLDRCRQVLALGGEGHTAAVHAADPEEIAPFAALPAGRILINTPALFGGMGFSAEVDPSFQLGTGTWSGSICSDNVTPLHLINIKRIAHEVRPWRTVYDPVEL

SEQ ID NO: 63 - protein, 459 aa, synthetic construct
MDADLKSIAAARRCAETAFEAYRQFLGTDPAQIDAIVQAMAEAIEPEAARLGRMAVEETGYGNAQDKRVKDLMNARGTAEWLRDVTTLGMLWRDDATKIAAFGEPMGVVAALIPVTNPSSTVIFKVLSAVKAGNAIVCAPHPRGVKTGNEVVRIMARVAEQMGAPKGLIQCLDHVTVQGTAELMKHRRTSVVMATGGPGMVRAAYSSGKPTLAVGAGNVPCYVHRSMAQELDEVAEMIIASKSFDWGTACVSEQAVIADPEIARELRAELKLKGGYFCTAPEADRLAKVIFTGSQAMNPERVGQAPEVLAELAGFSLPPRTKCLIAEETEIGWHRPLSAEKLNPVLAFYEARDSAHGIALAHGIATFEGWGHSAVIHAHDPQVVADFARVPTGRVLVNVPAIHGGAGYATDLEPSFMLGTGTWSGSITSDNVTALHLINIKRVAYGNRPWRDIYEEYGA

SEQ ID NO: 64 - protein, 463 aa, Serratia fonticola
MNTTELESLIRTILTEQLAPETASNQRAIAIFDSVDEAISAAHHAFLRFQQSPLKTRSAIISALREQLKPHLPVLAERGASETGMGNSADKLLKNLAALENTPGVEDLATTAITGDGGMVLFEYSPFGVIGAVAPSTNPTETIINNSISMLAAGNAVYFSPHPGAKNVSLDLIAMVEEIIFNSCGIRNLVVTVKEPSFAATQEMMAHDKIALLAITGGPGIVAMGMKSGKKVIGAGAGNPPCLVDETAELVKAAQDIVAGASFDYNLPCIAEKALIVVDSVADRLLQQMQAFDALLIAQPQDVDRLRQVCLTEGHANKDLVGKSPAELLAAAGLTCPAKLPRLLLVEVSGDDPLVTTEQLMPLLPVVRVKDFDTGLTLALQVEDGLHHTAVMHSQNVSRLNLAARRLQTSIFVKNGPSYAGIGVGGEGFTTFTIATPTGEGTTSARTFARQRRCVLTNAFSIR

SEQ ID NO: 65 - protein, 477 aa, synthetic construct
MNTENIEQAIRKILSEELSNPQSSTATNTTVPGKNGIFKTVNEAIAATKAAQENYADQPISVRNKVIDAIREGFRPYIEDMAKRIHDETGMGTVSAKIAKLNNALYNTPGPEILQPEAETGDGGLVMYEYAPFGVIGAVGPSTNPSETVIANAIMMLAGGNTLFFGAHPGAKNITRWTIEKLNELVADATGLHNLVVSLETPSIESVQEVMQHPDVAMLSITGGPAVVHQALISGKKAVGAGAGNPPAMVDATANIALAAHNIVDSAAFDNNILCTAEKEVVVEAAVKDELIMRMQQEGAFLVTDSADIEKLAQMTIGPKGAPDRKFVGKDATYILDQAGISYTGTPTLIILEAAKDHPLVTTEMLMPILPVVCCPDFDSVLATATEVEGGLHHTASIHSENLPHINKAAHRLNTSIFVVNGPTYCGTGVATNGAHSGASALTIATPTGEGTATSKTYTRRRRLNSPEGFSLRTWEA

SEQ ID NO: 66 - protein, 469 aa, Listeria innocua
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRESCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 67 - protein, 474 aa, Isosphaera pallida
MQPSQTEELIRSVVQQVLSQMGTVFPGGLPGVGSGRLGVFPTVDAAAHAARDAFQRFRGRPLADRRRAIDVIRKVVIEGAEELGSMELAETKIGRRDHKIEKLVSAGQKIPGVEYLRTDNVSGDLGITLTDYAPFGVVGAVTPVTHSLPTLAGNAINILAAGNTVVFHPHPSGVGVALEGVRRFNQGIREAIGLENLITIVESPTLESAQQLFDHREVDLLLVTGGPAVARAALSSRKRAIVAGPGNPPVVVDATACLDNAARSIVTGAAYDNNLLCIGEKQVFAPSNVLDRLMDRMARYGGHRLDARQIEALTAAAFKTGEGGQPTLNKELVGQDPAVLAAHAGLSIPSGVQLLFGETPADHPFVQLEQMMPFVPFVRTADLDQAIEQAHRSEHGYGHTAVLHSRDTATMTRMGRLMNCTIFVINGPCTAGLGGGGEGVLSYSIAGPTGEGVTTPLTFCRQQRTAVVGAMRFL

SEQ ID NO: 68 - protein, 467 aa, synthetic construct
MNPAELPHQVHESGANGVFDRIEDAIEAGYIAQLNYVKQFQLKDREKIITAIREAVIENKEKLAQMVFEETKLGRYEDKIAKHELVARKTPGTEDITTAAFSGDEGLTIIEQAPFGLVGAVTPVTNPTETIINNSISLLAAGNAVVLNVHPSSKVSCAFVVNLINQAIKDTGGPENLVSMVKDPTLETLNRIIESPKVKLLVGTGGPGMVKTLLKSGKKAIGAGAGNPPVIVDETADLKQAAKSIIEGASFDNNLLCIAEKELFVIDSVADDLIFHMLNEGAYMLDQQQLSKLMSFALEENVHQEAGGCSLDNKREYHVSKDWVGKDAVSFLRQLGIAHEEDIKLLICEVDFDHPFVQLEQMMPVFPIVRVGNLDEAIEMALLAEHGNRHTAIMHSKNVDHLTKFARAIETTIFVKNASSLAGVGFGGEGHTTMTIAGPTGEGITSAKTFTRQRRCVLAEGGFRIIG

SEQ ID NO: 69 - protein, 531 aa, Dongia mobilis
MTAIVEAILAGDQRATGEADMMLESAEWAAAAFARFDRARIARIARAAADVAYAHAGELADAAVAETGFGVAAHKKIKNELTSKALHDLYAAEDFCSARIRAELKMVELPRPAGIVFALTPSTNPVCSVYYKVLMALFTRNAIILSPHPMAKKCSVRAAKLMAEAAEAAGAPLGVIQVIEQPSLPLIEHVMKSPRVGVILATGGTPMVRAAYSSGNPAIGVGPGNAPVLVDDTADLNAAAKRIVESKSFDNSILCTNESVVIAVESIADRLLQSLKAAGAHVAKPEEVAALRELLFGRGTFNIDVLGKSAIEIGAKAGLKLPANTKIILAEIDRIGIDEPLSKEKLCPVLGFLRVPHAQAGITQARALVRLSGAGHSAAIHSKHAPTILAYGAAVKALRVVVNAPCSQGAAGFGTHLAPAFTIGTGYFGSSSVGENVGPQHLVNWTRIAYNADAAEAFGGFDGLDAWAGGPALALGREAPMTLGRLQEGAEAVQNPPPQPPGRAGDTDEMALMREEIRRMVLEELRGALRS

SEQ ID NO: 70 - protein, 486 aa, synthetic construct
MSGLQPDEIQAIIERVRRRVGDDGEPDGARLRGEQALEASPGVSAGGGVHAGVQDALSAAGRAFAAFDGAGLEARKRIISSVRQAMLEHGDRLAEMAQAETGLGRADDKTRKNRLVTTRTPGPEDLELDAETGDRGMNVTEFAPFGIVAAITPTTNPTSTVINNTIAIVSAGNAVVFNVHPNAKQVSAENVRLINEAIMRAGGPPDLVTTIAEPTIESAQEVMNHPDVRLLLVTGGPGVVREALKTDKRAVTAGPGNPPAVVDETADIEQAAADIVAGGSFDNNIICTDEKTTIAVDSIADPLVRAMERSGAYVLAEHELRRLERVIFRELGEPNKPGRINPQWVGQDVQAILAEIGVRVGPEVRMAVARVPNEHSLVWTEQMMPVMPVTSVRDVDQAIDLAVRSEHRFGHTASMHSSDVGRITRMGRAMNCSIFVANGPCYAGLGEGGEGFCSFSIATPTGDGLTRPQTFSRERRMTIVGSLRMV

SEQ ID NO: 71 - protein, 469 aa, synthetic construct
MQISEQVIQQLVEEVMNKLENNANQTAVTQLGQGVYPTVDEAVQAAKQANEALKKLSLATRVQMIENMRKTSIEHAEELASLAVQETGLGRVEDKRAKNILAAEKTPGMEDLPSTSYSGDNGLTIVEQAPLGVIGSITPTTNPAATMINNSLSMVAAGNVVVYNPHPSAKQVTLQTMKLLNEAIVEAGGPKNVLTSVIEPTLETSQQLMNHPEINALVVTGGGAVVKAAMSTGKKVIAAGPGNPPVVVDETANIKKAAKDIVFGASFDNNILCTAEKEVFVVQQVAQELKTEMLKNNAIELKAFQFEKLLKEILIEKDGKHYANKDYVGKDATVLLQAAGIQADKDTKLIIVEVSNDHPLVHTEMLMPILPIVKVSSVDEAIKLAKTAEKGNRHTAMMHSESITNLTKMAQTIEATIFVKNGPSVAGLGYESEGFTTLTIAGPTGEGLTSARTFTRQRRSVLVDGLRII

SEQ ID NO: 72 - protein, 639 aa, synthetic construct
MKPVLISLLEPLNKLSASEQMVLEKIMQPVSFSAGTYIFKEGSFADSCYILEEGIVRIETASESSSNFVVNYLEAGTIFGEISLLDKMPRSATAYAQTNIIAKKILIQELEILLETSPRILICLWEMFRKAAPLGVRSLNELTIKKNHFVINPEVEVMLDKALLAQKEIQTWSEERIDALLLVIASSIAQHAESLATATVKATRVGDILDKVTKNKIASLGIYRSLVGKSGCGFISNNKINNVCEIASPMGIIFGMIPMTNPVATAVFKALICIKSRNALILSFPLSTKNVGTLVCEIIQEALIRQDAPVELIQWLKHRSSREQTEILMKHKNVSLILATGGASMVKAAYSSGTPAIGVGPANTPTLICADANIQHAARTIIVSKSFDNGLICGSEHNLIVDARVRKNFIKVLEQEGAAILTHEEKSYFSTVAFEDKSNRLQAKIIGQSAAKIAVMANIKRDYHIKLIIVPNEFLSLDNPYAYEKMAPILSLFTVQNEIEGIDLCRSILQIEGKGHTAIIHTKNKAVVRRFGLEMPASRILVNSPGVHGIIGLTSALDPSFTLGCGTFGGNSTTDNVTYSNLLNLKRVAYYQAPKFLDPEIFKNTYPQWLLQLLNLLNFLKLNALVTFISQRVSIQLRR

SEQ ID NO: 73 - protein, 262 aa, Escherichia coli
MMEFILSHVEKGVMTLTLNRPERLNSFNDEMHAQLAECLKQVERDDTIRCLLLTGAGRGFCAGQDLNDRNVDPTGPAPDLGMSVERFYNPLVRRLAKLPKPVICAVNGVAAGAGATLALGGDIVIAARSAKFVMAFSKLGLIPDCGGTWLLPRVAGRARAMGLALLGNQLSAEQAHEWGMIWQVVDDETLADTAQQLARHLATQPTFGLGLIKQAINSAETNTLDTQLDLERDYQRLAGRSADYREGVSAFLAKRSPQFTGK

SEQ ID NO: 74 - protein, 297 aa, Escherichia coli
MKQQGTTLPANNHTLKQYAFFAGMLSSLKKQKWRKGMSESLHLTRNGSILEITLDRPKANAIDAKTSFEMGEVFLNFRDDPQLRVAIITGAGEKFFSAGWDLKAAAEGEAPDADFGPGGFAGLTEIFNLDKPVIAAVNGYAFGGGFELALAADFIVCADNASFALPEAKLGIVPDSGGVLRLPKILPPAIVNEMVMTGRRMGAEEALRWGIVNRVVSQAELMDNARELAQQLVNSAPLAIAALKEIYRTTSEMPVEEAYRYIRSGVLKHYPSVLHSEDAIEGPLAFAEKRDPVWKGR

SEQ ID NO: 75 - protein, 255 aa, Escherichia coli
MSELIVSRQQRVLLLTLNRPAARNALNNALLMQLVNELEAAATDTSISVCVITGNARFFAAGADLNEMAEKDLAATLNDTRPQLWARLQAFNKPLIAAVNGYALGAGCELALLCDVVVAGENARFGLPEITLGIMPGAGGTQRLIRSVGKSLASKMVLSGESITAQQAQQAGLVSDVFPSDLTLEYALQLASKMARHSPLALQAAKQALRQSQEVALQAGLAQERQLFTLLAATEDRHEGISAFLQKRTPDFKGR

SEQ ID NO: 76 - protein, 285 aa, Escherichia coli
MIYPDEAMLYAPVEWHDCSEGFEDIRYEKSTDGIAKITINRPQVRNAFRPLTVKEMIQALADARYDDNIGVIILTGAGDKAFCSGGDQKVRGDYGGYKDDSGVHHLNVLDFQRQIRTCPKPVVAMVAGYSIGGGHVLHMMCDLTIAADNAIFGQTGPKVGSFDGGWGASYMARIVGQKKAREIWFLCRQYDAKQALDMGLVNTVVPLADLEKETVRWCREMLQNSPMALRCLKAALNADCDGQAGLQELAGNATMLFYMTEEGQEGRNAFNQKRQPDFSKFKRNP

SEQ ID NO: 77 - protein, 729 aa, Escherichia coli
MLYKGDTLYLDWLEDGIAELVFDAPGSVNKLDTATVASLGEAIGVLEQQSDLKGLLLRSNKAAFIVGADITEFLSLFLVPEEQLSQWLHFANSVFNRLEDLPVPTIAAVNGYALGGGCECVLATDYRLATPDLRIGLPETKLGIMPGFGGSVRMPRMLGADSALEIIAAGKDVGADQALKIGLVDGVVKAEKLVEGAKAVLRQAINGDLDWKAKRQPKLEPLKLSKIEATMSFTIAKGMVAQTAGKHYPAPITAVKTIEAAARFGREEALNLENKSFVPLAHTNEARALVGIFLNDQYVKGKAKKLTKDVETPKQAAVLGAGIMGGGIAYQSAWKGVPVVMKDINDKSLTLGMTEAAKLLNKQLERGKIDGLKLAGVISTIHPTLDYAGFDRVDIVVEAVVENPKVKKAVLAETEQKVRQDTVLASNTSTIPISELANALERPENFCGMHFFNPVHRMPLVEIIRGEKSSDETIAKVVAWASKMGKTPIVVNDCPGFFVNRVLFPYFAGFSQLLRDGADFRKIDKVMEKQFGWPMGPAYLLDVVGIDTAHHAQAVMAAGFPQRMQKDYRDAIDALFDANRFGQKNGLGFWRYKEDSKGKPKKEEDAAVEDLLAEVSQPKRDFSEEEIIARMMIPMVNEVVRCLEEGIIATPAEADMALVYGLGFPPFHGGAFRWLDTLGSAKYLDMAQQYQHLGPLYEVPEGLRNKARHNEPYYPPVEPARPVGDLKTA

SEQ ID NO: 78 - protein, 261 aa, Lactobacillus plantarum
MATLGANASLYSEQHRITYYECDRTGRATLTTLIDIAVLASEDQSDALGLTTEMVQSHGVGWVVTQYAIDITRMPRQDEVVTIAVRGSAYNPYFAYREFWIRDADGQQLAYITSIWVMMSQTTRRIVKILPELVAPYQSEVVKRIPRLPRPISFEATDTTITKPYHVRFFDIDPNRHVNNAHYFDWLVDTLPATFLLQHDLVHVDVRYENEVKYGQTVTAHANILPSEVADQVTTSHLIEVDDEKCCEVTIQWRTLPEPIQ

SEQ ID NO: 79 - protein, 188 aa, Arabidopsis thaliana
MIRVTGTAAPAMSVVFPTSWRQPVMLPLRSAKTFKPHTFLDLKGGKEMSEFHEVELKVRDYELDQFGVVNNAVYANYCQHGMHEFLESIGINCDEVARSGEALAISELTMNFLAPLRSGDKFVVKVNISRTSAARIYFDHSILKLPNQEVILEAKATVVWLDNKHRPVRIPSSIRSKFVHFLRQNDTV

SEQ ID NO: 80 - protein, 246 aa, Lactobacillus brevis
MAANEFSETHRVVYYEADDTGQLTLAMLINLFVLVSEDQNDALGLSTAFVQSHGVGWVVTQYHLHIDELPRTGAQVTIKTRATAYNRYFAYREYWLLDDAGQVLAYGEGIWVTMSYATRKITTIPAEVMAPYHSEEQTRLPRLPRPDHFDEAVNQTLKPYTVRYFDIDGNGHVNNAHYFDWMLDVLPATFLRAHHPTDVKIRFENEVQYGHQVTSELSQAAALTTQHMIKVGDLTAVKATIQWDNR

SEQ ID NO: 81 - protein, 237 aa, synthetic construct
MIYMAYQYRSRIRYSEIGEDKKLTLPGLVNYFQDCSTFQSEALGIGLDTLGARQRAWLLASWKIVIDRLPRLGEEVVTETWPYGFKGFQGNRNFRMLDQEGHTLAAAASVWIYLNVESGHPCRIDGDVLEAYELEEELPLGPFSRKIPVPEESTERDSFLVMRSHLDTNHHVNNGQYILMAEEYLPEGFKVKQIRVEYRKAAVLHDTIVPFVCTEPQRCTVSLCGSDEKPFAVVEFS

SEQ ID NO: 82 - protein, 299 aa, synthetic construct
MFDRKSKRPSMLMDSFGLERVVQDGLVFRQSFSIRSYEICADRTASMETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVSNVKYIGWILESMPTEVLETQELCSLTLEYRRECGRDSVLESVTSMDPSKVGDRFQYRHLLRLEDGADIMKGRTEWRPKNAGTNGAISTGKT

SEQ ID NO: 83 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 84 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 85 - protein, 383 aa, Escherichia coli
MAASTFFIPSVNVIGADSLTDAMNMMADYGFTRTLIVTDNMLTKLGMAGDVQKALEERNIFSVIYDGTQPNPTTENVAAGLKLLKENNCDSVISLGGGSPHDCAKGIALVAANGGDIRDYEGVDRSAKPQLPMIAINTTAGTASEMTRFCIITDEARHIKMAIVDKHVTPLLSVNDSSLMIGMPKSLTAATGMDALTHAIEAYVSIAATPITDACALKAVTMIAENLPLAVEDGSNAKAREAMAYAQFLAGMAFNNASLGYVHAMAHQLGGFYNLPHGVCNAVLLPHVQVFNSKVAAARLRDCAAAMGVNVTGKNDAEGAEACINAIRELAKKVDIPAGLRDLNVKEEDFAVLATNALKDACGFTNPIQATHEEIVAIYRAAM

SEQ ID NO: 86 - protein, 382 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 87 - protein, 362 aa, Escherichia coli
MPHNPIRVVVGPANYFSHPGSFNHLHDFFTDEQLSRAVWIYGKRAIAAAQTKLPPAFGLPGAKHILFRGHCSESDVQQLAAESGDDRSVVIGVGGGALLDTAKALARRLGLPFVAVPTIAATCAAWTPLSVWYNDAGQALHYEIFDDANFMVLVEPEIILNAPQQYLLAGIGDTLAKWYEAVVLAPQPETLPLTVRLGINNAQAIRDVLLNSSEQALSDQQNQQLTQSFCDVVDAIIAGGGMVGGLGDRFTRVAAAHAVHNGLTVLPQTEKFLHGTKVAYGILVQSALLGQDDVLAQLTGAYQRFHLPTTLAELEVDINNQAEIDKVIAHTLRPVESIHYLPVTLTPDTLRAAFKKVESFKA

SEQ ID NO: 88 - protein, 362 aa, Escherichia coli
MERIVVTLGERSYPITIASGLFNEPASFLPLKSGEQVMLVTNETLAPLYLDKVRGVLEQAGVNVDSVILPDGEQYKSLAVLDTVFTALLQKPHGRDTTLVALGGGVVGDLTGFAAASYQRGVRFIQVPTTLLSQVDSSVGGKTAVNHPLGKNMIGAFYQPASVVVDLDCLKTLPPRELASGLAEVIKYGIILDGAFFNWLEENLDALLRLDGPAMAYCIRRCCELKAEVVAADERETGLRALLNLGHTFGHAIEAEMGYGNWLHGEAVAAGMVMAARTSERLGQFSSAETQRIITLLKRAGLPVNGPREMSAQAYLPHMLRDKKVLAGEMRLILPLAIGKSEVRSGVSHELVLNAIADCQSA

SEQ ID NO: 89 - protein, 251 aa, Bacillus subtilis
MAEQNKCALVTGSSRGVGKAAAIRLAENGYNIVINYARSKKAALETAEEIEKLGVKVLVVKANVGQPAKIKEMFQQIDETFGRLDVFVNNAASGVLRPVMELEETHWDWTMNINAKALLFCAQEAAKLMEKNGGGHIVSISSLGSIRYLENYTTVGVSKAALEALTRYLAVELSPKQIIVNAVSGGAIDTDALKHFPNREDLLEDARQNTPAGRMVEIKDMVDTVEFLVSSKADMIRGQTIIVDGGRSLLV